(19) United States
(12) Patent Application Publication
(10) Pub. No.: US 2012/0195885 A1
(43) Pub. Date: Aug. 2, 2012

(54) COMPOSITIONS CONTAINING GLYCOSYLATED ANTIBODIES AND USES THEREOF

(75) Inventors: Ivan R.S. Correia, Winchester, MA (US); Taro Fujimori, Shrewsbury, MA (US); Matthew W. Hruska, Lindenhurst, IL (US); Susan Kaye Paulson, Downers Grove, IL (US)

(73) Assignee: ABBOTT LABORATORIES, Abbott Park, IL (US)

(21) Appl. No.: 13/359,250

(22) Filed: Jan. 26, 2012

Related U.S. Application Data
(60) Provisional application No. 61/437,107, filed on Jan. 28, 2011.

(51) Int. Cl. A61K 39/395

(52) U.S. Cl. 424/133.1

(57) ABSTRACT

The present invention provides compositions of antibodies, e.g., human antibodies, of varying glycosylation structures that serve to achieve desired rates of serum clearance. The invention also provides methods for modulating the pharmacokinetics of antibodies, e.g., human antibodies, and therapeutic compositions containing such antibodies. These methods rely on varying the glycosylation structures of the antibodies, e.g., human antibodies, to achieve desired rates of serum clearance.

### RELATED APPLICATIONS

This application claims priority to U.S. Provisional Patent Application Ser. No. 61/437,107, filed on Jan. 28, 2011, the entire contents of which are incorporated herein by reference.

### BACKGROUND OF THE INVENTION

Antibody therapeutics are widespread. There are approximately two dozen therapeutic antibodies in the market. Antibodies produced using recombinant techniques may be glycosylated and, thus, exist as numerous glycoforms which can influence the therapeutic efficacy of the antibody by influencing, e.g., antibody effector functions, such as antibody-dependent cellular cytotoxicity and complement-dependent toxicity (Jefferis, R. (2009), Trends in Pharmacological Sciences 30(7): 356-362).

Several preclinical studies have noted some effect of glycosylation and glycoform abundance on the pharmacokinetics of recombinant antibodies, however no effect of glycosylation and glycoform abundance on the pharmacokinetics of recombinant antibodies have been identified in clinical studies (see, e.g., Chen et al. (2007) Glycobiology, 19(3): 240-249; Jones et al. (2007) Glycobiology, 17(5) 529-540; Kanda et al. (2006) Glycobiology 17(1): 104-118; Keck et al. (2008) Biologicals 36: 49-60; Millward et al. (2008) Biologicals 36: 41-47; Newkirk et al. (1996) Clin Exp Immunol 106: 259-264; Wawrzynczak et al. (1992) Molecular Immunology 29(2): 213-220; Wright et al (1994) J Exp Med 180: 1087-1096; Zhou (2008) Biotechnology and Bioengineering 99(3): 652-665; Chen et al. (2007) Glycobiology, 19(3): 240-249).

Accordingly, there is a need to better characterize the glycoforms of antibodies and the associated effect on the pharmacokinetics of gylcosylated antibodies.

### SUMMARY OF THE INVENTION

The present invention is based, at least in part, on the discovery of a relationship between the level and type of glycoforms of a human antibody and the rate of serum clearance of the antibody. More specifically, eight glycoforms of a human anti-IL-12/IL-23 p40 antibody (ABT-874) have been identified in a composition of ABT-874 following injection of the composition into a human subject. Structural analyses of the eight glycoforms permitted the separation of the glycofoms into two groups, the oligomannose-type structures, and the fucosylated bianntenary oligosaccharide-type structures which was further supported by pharmacokinetic analysis of the 8 glycoforms.

Population pharmacokinetic modeling of the two groups demonstrated that, although the oligomannose-type structures of ABT-874 have an approximately 40% greater clearance rate than the fucosylated bianntenary oligosaccharide-type structures of ABT-874, the overall clearance rate of ABT-874 is not affected because the percentage of the oligomannose-type structures in the ABT-874 compostion is about 10% compared to 90% of the fucosylated bianntenary oligosaccharide-type structures.

Population pharmacokinetic modeling of the two groups further demonstrated that increasing the level of oligomannose-type structures in the ABT-874 compostion to approximately 30% of the total level of oligosaccharide structures does not have an impact on the pharmacokinetics or rate of serum clearance of the antibody, or antigen-binding fragment thereof.

Accordingly, in one aspect, the invention provides compositions comprising a human antibody, or antigen binding portion thereof. The compositions include a first level of the antibody, or antigen binding portion thereof, which is glycosylated at an N-linked glycosylation site on the Fc region with an oligomannose-type structure, and a second level of the antibody, or antigen binding portion thereof, which is glycosylated at the N-linked glycosylation site on the Fc region with a fucosylated biantennary oligosaccharide-type structure, wherein the composition exhibits a desired rate of serum clearance.

In one embodiment, the N-linked glycosylation site is an asparagine residue on the Fc region of the antibody, such as Asn 297.

In one embodiment, the oligomannose-type structure is independently selected from the group consisting of M5, M6, M7, M8, and M9.

In one embodiment, the fucosylated biantennary oligosaccharide-type structure is independently selected from the group consisting of NGA2F, NA1F, NA2F, NGA2F-GlcNAc, and NA1F-GlcNAc.

In one embodiment, the first level is about 0-100%. In another embodiment, the first level is about 10-30%. In yet another embodiment, the first level is selected from the group consisting of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 8%, 99%, and about 100%.

In one embodiment, the second level is about 0-100%. In another embodiment, the second level is about 70-90%. In yet another embodiment, the second level is selected from the group consisting of 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 8%, 99%, and 100%.

The desired rate of serum clearance may be a rapid rate of serum clearance. In one embodiment, the first level is greater than about 50%. In another embodiment, the first level is greater than about 30%. In one embodiment, the first level is about 51-100%. In another embodiment, the first level is about 31-100%.

The desired rate of serum clearance may be a slow rate of serum clearance. In one embodiment, the first level is about 0-50%. In another embodiment, the first level is about 10-30%.

The antibody, or antigen binding portion thereof, may comprise a λ light chain.

The antibody, or antigen binding portion thereof, may comprise a heavy chain constant region selected from the group consisting of IgG1, IgG2, IgG3 and IgG4 constant regions. In one embodiment, the heavy chain constant region is an IgG 1 heavy chain. In another embodiment, the antibody, or antigen binding portion thereof, comprises an IgG1 heavy chain constant region and a λ light chain.

The antibody, or antigen binding portion thereof, may be produced in a mammalian cell, a CHO cell, or a myeloma cell line.

The antibody, or antigen binding portion thereof, may be an anti-IL-12 antibody, an anti-IL-23 antibody, or ABT-874 or a fragment thereof.

In one embodiment, the antibody, or antigen binding portion thereof, comprises a heavy chain CDR3 comprising the amino acid sequence of SEQ ID NO: 25 and a light chain CDR3 comprising the amino acid sequence of SEQ ID NO: 26. In one embodiment, the human antibody, or antigen binding portion thereof, further comprises a heavy chain CDR2 comprising the amino acid sequence of SEQ ID NO: 27 and a light chain CDR2 comprising the amino acid sequence of SEQ ID NO: 28. In another embodiment, the human antibody, or antigen binding portion thereof, further comprises a heavy chain CDR1 comprising the amino acid sequence of SEQ ID NO: 29 and a light chain CDR1 comprising the amino acid sequence of SEQ ID NO: 30.

In one embodiment, the antibody, or antigen binding portion thereof, comprises a heavy chain variable region comprising the amino acid sequence of SEQ ID NO: 31, and a light chain variable region comprising the amino acid sequence of SEQ ID NO: 32.

In one embodiment, the antibody, or antigen binding portion thereof, is an antibody, or fragment thereof, selected from the group consisting of CNT01275, tositumomab, WRI-170, WO1, TNF-H9G1, THY-32, THY-29, TEL16, TEL14, Tel13, SM1, S1-1, RSP4, RH-14, RF-TS7, RF-SJ2, RF-SJ1, RF-AN, PR-TS2, PR-TS1, PR-SJ2, PR-SJ1, PHOX15, PAG-1, OG-31, NO. 13, NM3E2 SCFV, MUC1-1, MN215, MC116, MAD-2, MAB67, MAB63, MAB60, MAB59, MAB57, MAB56, MAB111, MAB107, L3055-BL, K6H6, K6F5, K5G5, K5C7, K5B8, K4B8, JAC-10, HUC, HMST-1, HIH2, HIH10, HBW4-1, HBP2, HA1, H6-3C4, H210, GP44, GG48, GG3, GAD-2, FOM-A, FOM-1, FOG1-A3, FOG-B, DPC, DPA, DOB1, DO1, CLL001, CLL-249, CD4-74, CB-201, C304 RF, BSA3, BO3, B01, BEN-27, B-33, B-24, ANTI-TEST, ANTI-EST, ANTI-DIGB, ANTI-DIGA, AIG, 9604, 448.9G.F1, 33.H11, 32.B9, 24A5, 1B9/F2, 13E10, 123AV16-1, 11-50, and 1.32.

The compositions of the invention may further comprise an additional agent selected from the group consisting of a buffer, a polyol and a surfactant. In one embodiment, the buffer is selected from the group consisting of L-histidine, sodium succinate, sodium citrate, sodium phosphate and potassium phosphate. In one embodiment, the polyol is selected from the group consisting of mannitol and sorbitol. In one embodiment, the surfactant is selected from the group consisting of polysorbate 80, polysorbate 20 and BRIJ surfactants. In one embodiment, the compositions of the invention further comprise methionine.

The concentration of the antibody, or antigen binding portion thereof, in the compositions may be about 0.1-250 mg/ml.

The compositions of the invention may be suitable for parenteral administration, for intravenous injection or intravenous infusion, or for subcutaneous injection or intramuscular injection.

The compositions of the invention may further comprise an additional therapeutic agent. In one embodiment, the additional therapeutic agent is selected from the group consisting of budenoside, epidermal growth factor, corticosteroids, cyclosporin, sulfasalazine, aminosalicylates, 6-mercaptopurine, azathioprine, metronidazole, lipoxygenase inhibitors, mesalamine, olsalazine, balsalazide, antioxidants, thromboxane inhibitors, IL-1 receptor antagonists, anti-IL-10 monoclonal antibodies, anti-IL-6 monoclonal antibodies, growth factors, elastase inhibitors, pyridinyl-imidazole compounds, antibodies or agonists of TNF, LT, IL-1, IL-2, IL-6, IL-7, IL-8, IL-15, IL-16, IL-18, EMAP-II, GM-CSF, FGF, and PDGF, antibodies of CD2, CD3, CD4, CD8, CD25, CD28, CD30, CD40, CD45, CD69, CD90 or their ligands, methotrexate, cyclosporin, FK506, rapamycin, mycophenolate mofetil, leflunomide, NTHEs, ibuprofen, corticosteroids, prednisolone, phosphodiesterase inhibitors, adenosine agonists, antithrombotic agents, complement inhibitors, adrenergic agents, IRAK, NIK, IKK, p38, MAP kinase inhibitors, IL-1β converting enzyme inhibitors, TNFα converting enzyme inhibitors, T-cell signalling inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1RI, sIL-1RII, sIL-6R, antiinflammatory cytokines, IL-4, IL-10, IL-11, IL-13 and TGFβ. In another embodiment, the additional therapeutic agent is selected from the group consisting of anti-TNF antibodies and antibody fragments thereof, TNFR-Ig constructs, TACE inhibitors, PDE4 inhibitors, corticosteroids, budenoside, dexamethasone, sulfasalazine, 5-aminosalicylic acid, olsalazine, IL-1β converting enzyme inhibitors, IL-1ra, tyrosine kinase inhibitors, 6-mercaptopurines and IL-11. In yet another embodiment, the additional therapeutic agent is selected from the group consisting of corticosteroids, prednisolone, methylprednisolone, azathioprine, cyclophosphamide, cyclosporine, methotrexate, 4-aminopyridine, tizanidine, interferon-β1a, interferon-β1b, Copolymer 1, hyperbaric oxygen, intravenous immunoglobulin, clabribine, antibodies or agonists of TNF, LT, IL-1, IL-2, IL-6, IL-7, IL-8, IL-15, IL-16, IL-18, EMAP-II, GM-CSF, FGF, PDGF, antibodies to CD2, CD3, CD4, CD8, CD25, CD28, CD30, CD40, CD45, CD69, CD80, CD86, CD90 or their ligands, methotrexate, cyclosporine, FK506, rapamycin, mycophenolate mofetil, leflunomide, NTHEs, ibuprofen, corticosteroids, prednisolone, phosphodiesterase inhibitors, adenosine agonists, antithrombotic agents, complement inhibitors, adrenergic agents, IRAK, NIK, IKK, p38 or MAP kinase inhibitors, IL-1β converting enzyme inhibitors, TACE inhibitors, T-cell signalling inhibitors, kinase inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1RI, sIL-1RII, sIL-6R, sIL-13R, anti-P7s, p-selectin glycoprotein ligand (PSGL), antiinflammatory cytokines, IL-4, IL-10, IL-13 and TGFβ.

In another aspect, the present invention provides compositions comprising a human antibody, or antigen binding portion thereof. The compositions include 0-100% of the antibody, or antigen binding portion thereof, which is glycosylated at an N-linked glycosylation site on the Fc region with an oligomannose-type structure, and 0-100% of the antibody, or antigen binding portion thereof, which is glycosylated at the N-linked glycosylation site on the Fc region with a fucosylated biantennary oligosaccharide-type structure, wherein the composition exhibits a desired rate of serum clearance.

In yet another aspect, the present invention provides compositions comprising a human antibody, or antigen binding portion thereof. The compositions include about 10-30% of the antibody, or antigen binding portion thereof, which is glycosylated at an N-linked glycosylation site on the Fc region with an oligomannose-type structure, and about 70-90% of the antibody, or antigen binding portion thereof, which is glycosylated at the N-linked glycosylation site on the Fc region with a fucosylated biantennary oligosaccharide-type structure, wherein the composition exhibits a desired rate of serum clearance.

In one aspect, the present invention provides compositions comprising ABT-874, or antigen binding portion thereof. The copostions include about 0-100% of the ABT-874 is glycosylated at Asn 297 with an oligomannose structure that is independently selected from the group consisting of M5, M6, M7, M8 and M9, and about 0-100% of the ABT-874 is glycosylated at Asn 297 with a fucosylated biantennary oligosaccharide structure that is independently selected from the group consisting of NGA2F, NA1F, NA2F, NGA2F-GlcNAc, and NA1F-GlcNAc.

In another aspect, the present invention provides compositions comprising ABT-874, or antigen binding portion thereof. The copositions include about 10-30% of the ABT-874 is glycosylated at Asn 297 with an oligomannose structure that is independently selected from the group consisting of M5, M6, M7, M8 and M9, and about 70-90% of the ABT-874 is glycosylated at Asn 297 with a fucosylated biantennary oligosaccharide structure that is independently selected from the group consisting of NGA2F, NA1F, NA2F, NGA2F-GlcNAc, and NA1F-GlcNAc.

In one aspect, the present invention provides methods for modulating the pharmacokinetics of a composition comprising a human antibody, or antigen binding portion thereof. The methods include modulating a first level of the antibody that is glycosylated at an N-linked glycosylation site on the Fc region with an oligomannose-type structure, and modulating a second level of the antibody that is glycosylated at the N-linked glycosylation site on the Fc region with a fucosylated biantennary oligosaccharide-type structure, wherein the modulation of the first and second levels results in a desired rate of serum clearance, thereby modulating the pharmacokinetics of a composition comprising a human antibody, or antigen binding portion thereof.

The N-linked glycosylation site may be an asparagine residue on the Fc region of the antibody, such as Asn 297.

In one embodiment, the oligomannose-type structure is independently selected from the group consisting of M5, M6, M7, M8, and M9.

In one embodiment, the fucosylated biantennary oligosaccharide-type structure is independently selected from the group consisting of NGA2F, NA1F, NA2F, NGA2F-GlcNAc, and NA1F-GlcNAc.

In one embodiment, the first level is about 0-100%. In another embodiment, first level is about 10-30%. In one embodiment, the first level is selected from the group consisting of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 8%, 99%, and about 100%.

In one embodiment, the second level is about 0-100%. In another embodiment, the second level is about 70-90%. In yet another embodiment, the second level is selected from the group consisting of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 8%, 99%, and about 100%.

In one embodiment, the desired rate of serum clearance is a rapid rate of serum clearance. In one embodiment, the first level is greater than about 50%. In another embodiment, the first level is greater than about 30%. In one embodiment, the first level is about 51-100%. In another embodiment, the first level is about 31-100%.

In one embodiment, the desired rate of serum clearance is a slow rate of serum clearance. In one embodiment, the first level is about 0-100%. In one embodiment, the second level is about 10-30%.

The antibody, or antigen binding portion thereof, may comprise a λ light chain.

The antibody, or antigen binding portion thereof, may comprise a heavy chain constant region selected from the group consisting of IgG1, IgG2, IgG3, and IgG4 constant regions. In one embodiment, the heavy chain constant region is an IgG1. In one embodiment, the antibody, or antigen binding portion thereof, comprises an IgG1 heavy chain constant region and a λ light chain.

The antibody, or antigen binding portion thereof, may be produced in a mammalian cell, a CHO cell, or a myeloma cell line.

The antibody, or antigen binding portion thereof, may be an anti-IL-12 antibody, an anti-IL-23 antibody, or ABT-874 or a fragment thereof.

In one embodiment, the antibody, or antigen binding portion thereof, comprises a heavy chain CDR3 comprising the amino acid sequence of SEQ ID NO: 25 and a light chain CDR3 comprising the amino acid sequence of SEQ ID NO: 26. In one embodiment, the human antibody, or antigen binding portion thereof, further comprises a heavy chain CDR2 comprising the amino acid sequence of SEQ ID NO: 27 and a light chain CDR2 comprising the amino acid sequence of SEQ ID NO: 28. In another embodiment, the human antibody, or antigen binding portion thereof, further comprises a heavy chain CDR1 comprising the amino acid sequence of SEQ ID NO: 29 and a light chain CDR1 comprising the amino acid sequence of SEQ ID NO: 30. In one embodiment, the antibody, or antigen binding portion thereof, comprises a heavy chain variable region comprising the amino acid sequence of SEQ ID NO: 31, and a light chain variable region comprising the amino acid sequence of SEQ ID NO: 32.

In one embodiment, the antibody, or antigen binding portion thereof, is an antibody, or fragment thereof, selected from the group consisting of CNT01275, tositumomab, WRI-170, WO1, TNF-H9G1, THY-32, THY-29, TEL16, TEL14, Tel13, SM1, S1-1, RSP4, RH-14, RF-TS7, RF-SJ2, RF-SJ1, RF-AN, PR-TS2, PR-TS1, PR-SJ2, PR-SJ1, PHOX15, PAG-1, OG-31, NO. 13, NM3E2 SCFV, MUC1-1, MN215, MC116, MAD-2, MAB67, MAB63, MAB60, MAB59, MAB57, MAB56, MAB111, MAB107, L3055-BL, K6H6, K6F5, K5G5, K5C7, K5B8, K4B8, JAC-10, HUC, HMST-1, HIH2, HIH10, HBW4-1, HBP2, HA1, H6-3C4, H210, GP44, GG48, GG3, GAD-2, FOM-A, FOM-1, FOG1-A3, FOG-B, DPC, DPA, DOB1, DO1, CLL001, CLL-249, CD4-74, CB-201, C304 RF, BSA3, B03, B01, BEN-27, B-33, B-24, ANTI-TEST, ANTI-EST, ANTI-DIGB, ANTI-DIGA, AIG, 9604, 448.9G.F1, 33.H11, 32.B9, 24A5, 1B9/F2, 13E10, 123AV16-1, 11-50, and 1.32.

In one aspect, the present invention provides methods for modulating the pharmacokinetics of a composition comprising ABT-874, or an antigen-binding portion thereof. The methods include modulating a first level of ABT-874, or an antigen-binding fragment thereof, that is glycosylated at an N-linked glycosylation site on the Fc region with an oligomannose-type structure that is independently selected from the group consisting of M5, M6, M7, M8 and M9, and modulating a second level ABT-874, or an antigen-binding fragment thereof, that is glycosylated at the N-linked glycosylation site on the Fc region with a fucosylated biantennary oligosaccharide-type structure that is independently selected from the group consisting of NGA2F, NA1F, NA2F, NGA2F-GlcNAc, and NA1F-GlcNAc, wherein the modulation of the first and second levels results in a desired rate of serum clearance, thereby modulating the pharmacokinetics of a composition comprising ABT-874, or an antigen binding portion thereof.

Other features and advantages of the invention will be apparent from the following detailed description and claims.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIGS. 1A and 1B show the mean±SD of individual ABT-874 oligomannose-type (1A) and fucosylated bianntenary oligosaccharide-type structures (FBO) (1B) glycoforms over time following a single 700 mg IV infusion of ABT-874 to healthy subjects (log-linear scale)

FIGS. 2A and 2B show the mean±SD of serum concentration-time profiles for glycoform Group 1 (FBO) and Group 2 (oligomannose) on linear (2A) and log-linear (2B) scales following a single 700 mg IV infusion of ABT-874.

FIG. 3 The goodness-of-fit plots of the individual predicted ABT-874 concentrations versus the observed concentrations and the weight residuals versus time are presented.

The upper left panel shows the individual predicted concentrations (IPRE) versus observed concentrations of ABT-874 Group 1 (FBO) and the upper right panel shows the individual predicted concentrations (IPRE) versus observed concentrations of ABT-874 Group 2 (Oligomannoses).

For the concentration analysis vs. population predicted concentrations (PRED), the middle left panel shows the conditional weighted residuals (CWRES) of ABT-874 Group 1 (FBO), and the middle right panel shows the conditional weighted residuals (CWRES) of ABT-874 Group 2 (Oligomannoses).

For the concentration analysis vs. time, the lower left panel shows the conditional weighted residuals (CWRES) of ABT-874 Group 1 (FBO) and the lower left pane shows the conditional weighted residuals (CWRES) of ABT-874 Group 2 (Oligomannoses).

FIG. 4 shows visual predictive checks for ABT-874 Group 2 (Oligomannoses; Left) and Group 1 (FBO; Right). Solid line: median predicted concentrations; dotted lines: 5^{th} and 95^{th} percentiles of predicted concentrations; open circles: observed concentrations.

FIG. 5 shows simulated pharmacokinetic profiles of pure FBO (light gray) and oligomannose (medium gray) ABT-874 glycoforms (90% prediction interval).

FIG. 6 shows simulated pharmacokinetic profiles of test ABT-874 products with 70/30 FBO/Oligomannose (left) and 60/40 (right) plotted with reference product (90/10)

FIG. 7 shows simulated 90% confidence interval for AUC_{0-28d} ratio of test to reference (90/10) compositions from each 1000 replicated bioequivalence studies at different glycoform compositions.

FIG. 8 shows simulated 90% confidence interval for Cₘₐₓ ratio of test to reference (90/10) compositions from each 1000 replicated bioequivalence studies at different glycoform compositions.

FIG. 9 shows percentages of study replicates not meeting bioequivalence criteria (0.80-1.25) using 90/10 composition as reference. The upper left panel shows studies not meeting AUC. The upper right panel shows studies not meeting Cₘₐₓ. The lower panel shows studies not meeting AUC or Cₘₐₓ.

### DETAILED DESCRIPTION OF THE INVENTION

The present invention is based, at least in part, on the discovery of a relationship between the level and type of glycoforms of a human antibody and the rate of serum clearance of the antibody. More specifically, eight glycoforms of a human anti-IL-12/IL-23 p40 antibody (ABT-874) have been identified in a compositions of ABT-874 following administration to a human subject. Structural analyses of the eight glycoforms permitted the separation of the glycofoms into two groups, the oligomannose-type structures, and the fucosylated bianntenary oligosaccharide-type structures which was further supported by pharmacokinetic analysis of the 8 glycoforms.

Population pharmacokinetic modeling of the two groups demonstrated that, although the oligomannose-type structures of ABT-874 have an approximately 40% greater clearance rate than the fucosylated bianntenary oligosaccharide-type structures of ABT-874, the overall clearance rate of ABT-874 is not affected because the percentage of the oligomannose-type structures in the ABT-874 compostion is about 10% compared to 90% of the fucosylated bianntenary oligosaccharide-type structures.

Population pharmacokinetic modeling of the two groups further demonstrated that increasing the level of oligomannose-type structures in the ABT-874 compostion to approximately 30% of the total level of oligosaccharide structures does not have an impact on the pharmacokinetics or rate of serum clearance of the antibody, or antigen-binding fragment thereof.

Accordingly, the present invention provides compositions of antibodies, and antigen-binding fragments thereof, containing varying levels of glycoforms in order to achieve desired rates of serum clearance. In addition, the present invention provides methods for modulating the pharmacokinetics of human antibodies and therapeutic compositions involving human antibodies in order to achieve desired rates of serum clearance.

### I. DEFINITIONS

The articles “a” and “an” are used herein to refer to one or to more than one (i.e., to at least one) of the grammatical object of the article. By way of example, “an element” means one element or more than one element.

Most naturally occurring peptides (or proteins) comprise carbohydrate or saccharide moieties attached to the peptide via specific linkages to a select number of amino acids along the length of the primary peptide chain. Thus, many naturally occurring peptides are termed “glycopeptides” or “glycoproteins” or are referred to as “glycosylated” proteins or peptides.

The term “glycoform” refers an isoform of a protein, e.g., an antibody, that differs only with respect to the number and/or type of attached glycan(s). Glycoproteins often consist of a number of different glycoforms.

The predominant sugars found on glycoproteins are glucose, galactose, mannose, fucose, N-acetylgalactosamine (“GalNAc”), N-acetylglucosamine (“GlcNAc”) and sialic acid (e.g., N-acetylneuraminic acid (“NANA” or “NeuAc”, where “Neu” is neuraminic acid) and “Ac” refers to “acetyl”). The processing of the sugar groups occurs co-translationally in the lumen of the ER and continues in the Golgi apparatus for N-linked glycoproteins.

The oligosaccharide structure attached to the peptide chain is known as a “glycan” molecule. The glycan structures found in naturally occurring glycopeptides are typically divided into two classes, “N-linked glycans” or N-linked oligosaccharides” and “O-linked glycans” or O-linked oligosaccharides”.

Peptides comprising “O-linked glycans” have a saccharide attached to the hydroxy oxygen of serine, threonine, tyrosine, hydroxylysine, and or hydroxyproline residue in the primary protein.

Peptides expressed in eukaryotic cells typically comprise N-glycans. “N-glycans” are N-glycosylated at an amide nitrogen of an asparagine or an arginine residue in a protein via an N-acetylglucosamine residue. These “N-linked glycosylation sites” occur in the peptide primary structure containing, for example, the amino acid sequence asparagine-X-serine/threonine, where X is any amino acid residue except proline and aspartic acid.

Techniques for the determination of glycan primary structure are well known in the art and are described in detail, for example, in Montreuil, “Structure and Biosynthesis of Glycopeptides” In Polysaccharides in Medicinal Applications, pp. 273-327, 1996, Eds. Severian Damitriu, Marcel Dekker, NY. It is therefore a routine matter for one of ordinary skill in the art to isolate a population of peptides produced by a cell and determine the structure(s) of the glycans attached thereto. For example, efficient methods are available for (i) the splitting of glycosidic bonds either by chemical cleavage such as hydrolysis, acetolysis, hydrazinolysis, or by nitrous deamination; (ii) complete methylation followed by hydrolysis or methanolysis and by gas-liquid chromatography and mass spectroscopy of the partially methylated monosaccharides; and (iii) the definition of anomeric linkages between monosaccharides using exoglycosidases, which also provide insight into the primary glycan structure by sequential degradation. Fluorescent labeling and subsequent high performance liquid chromatography (HPLC), e.g., normal phase HPLC (NP-HPLC), mass spectroscopy and nuclear magnetic resonance (NMR) spectrometry, e.g., high field NMR, may also be used to determine glycan primary structure.

Kits and equipment for carbohydrate analysis are also commercially available. Fluorophore Assisted Carbohydrate Electrophoresis (FACE) is available from Glyko, Inc. (Novato, Calif.). In FACE analysis, glycoconjugates are released from the peptide with either Endo H or N-glycanase (PNGase F) for N-linked glycans, or hydrazine for Ser/Thr linked glycans. The glycan is then labeled at the reducing end with a fluorophore in a non-structure discriminating manner. The fluorophore labeled glycans are then separated in polyacrylamide gels based on the charge/mass ratio of the saccharide as well as the hydrodynamic volume. Images are taken of the gel under UV light and the composition of the glycans is determined by the migration distance as compared with the standards. Oligosaccharides can be sequenced in this manner by analyzing migration shifts due to the sequential removal of saccharides by exoglycosidase digestion.

All N-linked oligosaccharides have a common “pentasaccharide core” of Man₃GlcNAc₂. (“Man” refers to mannose; “Glc” refers to glucose; “NAc” refers to N-acetyl; and “GlcNAc” refers to N-acetylglucosamine). The pentasaccharide core is also referred to as the “trimannose core” or the “paucimannose core”.

N-glycans differ with respect to the presence of, and/or in the number of branches (also called “antennae”) comprising peripheral sugars such as N-acetylglucosamine, galactose, N-acetylgalactosamine, N-acetylneuraminic acid, fucose and sialic acid that are added to the Man₃GlcNAc₂ core structure. Optionally, this structure may also contain a core fucose molecule and/or a xylose molecule. For a review of standard glycobiology nomenclature see, Essentials of Glycobiology Varki et al. eds., 1999, CSHL Press, the contents of which are incorporated herein by reference.

N-glycans are classified according to their branched constituents (e.g., oligomannose-type, complex, or hybrid). An “oligomannose-type” or “high mannose-type” N-glycan has five or more mannose residues.

A “complex-type” N-glycan typically has at least one GlcNAc attached to the 1,3 mannose arm and at least one GlcNAc attached to the 1,6 mannose arm of a pentasaccharide core. Complex-type N-glycans may also have galactose (“Gal”) or N-acetylgalactosamine residues that are optionally modified with sialic acid or derivatives, e.g., N-acetyl neuraminic acid. Complex-type N-glycans may also have intrachain substitutions comprising “bisecting” GlcNAc, and core fucose (“Fuc”). Complex N-glycans may also have multiple antennae on the pentasaccharide core and are, therefore, also referred to as “multiple antennary-type glycans.”

A “hybrid-type” N-glycan comprises at least one GlcNAc on the terminal of the 1,3 mannose arm of the pentasaccharide core and zero or more mannoses on the 1,6 mannose arm of the trimannose core.

In one embodiment, a human antibody, or antigen-binding fragment thereof, present within the compositions of the invention and/or suitable for use in the claimed methods comprises an oligomannose-type structure. In another embodiment, a human antibody, or antigen-binding fragment thereof, present within the compositions of the invention and/or suitable for use in the claimed methods comprises a multiple antennary-type structure. In another embodiment, a human antibody, or antigen-binding fragment thereof, present within the compositions of the invention and/or suitable for use in the claimed methods comprises a hybrid-type structure. In yet another embodiment, a human antibody, or antigen-binding fragment thereof, present within the compositions of the invention and/or suitable for use in the claimed methods comprises an N-glycan structure independently selected from the group consisting of an oligomannose-type structure, a multiple antennary-type structure, and a hybrid-type structure.

The oligomannose-type structures that may be present within the compositions of the invention and/or may be used in the methods of the invention are referred to herein as “M5”, “M6”, “M7,” “M8,” and “M9.”

In one embodiment, an M5 oligomannose-type structure has the structure (I):

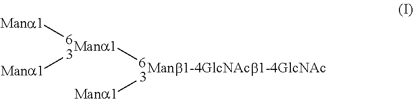

In one embodiment, an M6 oligomannose-type structure has the structure (II):

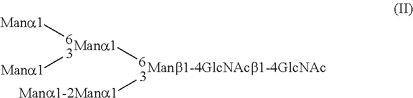

In one embodiment, an M7 oligomannose-type structure has the structure (III):

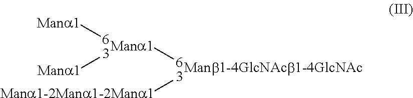

In another embodiment, an M7 oligomannose-type structure has the structure (IV):

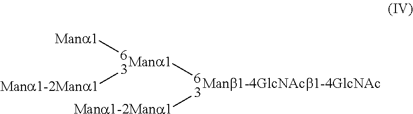

In another embodiment, an M7 oligomannose-type structure has the structure (V):

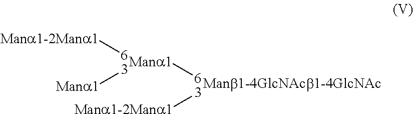

In one embodiment, an M8 oligomannose-type structure has the structure (VI):

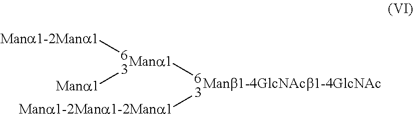

In another embodiment, an M8 oligomannose-type structure has the structure (VII):

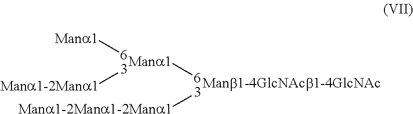

In another embodiment, an M8 oligomannose-type structure has the structure (VIII):

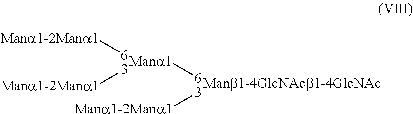

In one embodiment, an M9 oligomannose-type structure has the structure (IX):

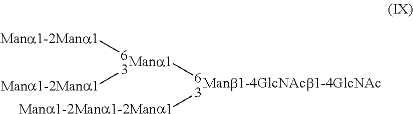

In one embodiment, the oligomannose-type structures that may be present within the compositions of the invention and/or may be used in the methods of the invention are independently selected from the group consisting of M5, M6, M7, M8, and M9.

In one embodiment, a multiple antennary-type structure that may be present within the compositions of the invention and/or may be used in the methods of the invention is a “bianntennary oligosaccharide-type structure”. A “bianntennary oligosaccharide-type structure” is an N-linked glycan having two branches or arms, and a core fucose with zero, one or two glactose additions on the arms. In one embodiment, a “bianntennary oligosaccharide-type structure” that may be present within the compositions of the invention and/or may be used in the methods of the invention is bisected. In one embodiment, a “bianntennary oligosaccharide-type structure” that may be present within the compositions of the invention and/or may be used in the methods of the invention is a “fucosylated bianntennary oligosaccharide-type structure”, e.g., comprises a core-substituted with fucose.

In one embodiment, a “fucosylated bianntennary oligosaccharide-type structure” that may be present within the compositions of the invention and/or may be used in the methods of the invention is an “asialo, fucosylated bianntennary oligosaccharide-type structure”, also referred to as an “asialo, bigalactosylated biantennary, core-substituted with fucose”, referred to herein as “NA2F.”

In another embodiment, a “fucosylated bianntennary oligosaccharide-type structure” that may be present within the compositions of the invention and/or may be used in the methods of the invention is a asialo, agalacto, fucosylated bianntennary oligosaccharide-type structure, also referred to as an asialo, agalacto-, biantennary, core-substituted with fucose, referred to herein as “NGA2F.”

In another embodiment, a a “fucosylated bianntennary oligosaccharide-type structure” that may be present within the compositions of the invention and/or may be used in the methods of the invention is a asialo, fucosylated bianntennary oligosaccharide-type structure, also referred to as asialo, monogalactosylated biantennary, core-substituted with fucose, referred to herein as “NA1F.”

In another embodiment, a a “fucosylated bianntennary oligosaccharide-type structure” that may be present within the compositions of the invention and/or may be used in the methods of the invention is a asialo, agalacto, fucosylated biantennary, minus a bisecting N-acetylglucosamine oligosaccharide-type structure, also referred to as asialo, agalacto-, biantennary, core-substituted with fucose minus a bisecting N-acetylglucosamine, referred to herein as “NGA2F-GlcNAc.”

In yet another embodiment, a a “fucosylated bianntennary oligosaccharide-type structure” that may be present within the compositions of the invention and/or may be used in the methods of the invention is a asialo, monogalacto, fucosylated biantennary, minus a bisecting N-acetylglucosamine oligosaccharide-type structure, also referred to as asialo, monogalactosylated biantennary, core-substituted with fucose minus a bisecting N-acetylglucosamine, referred to herein as “NA1F-GlcNAc.”

In one embodiment, an NA2F fucosylated biantennary oligosaccharide-type structure has the structure (X):

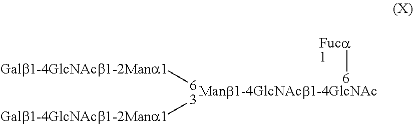

In one embodiment, an NGA2F fucosylated biantennary oligosaccharide-type structure has the structure (XI):

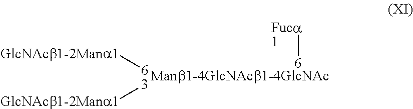

In one embodiment, an NA1F fucosylated biantennary oligosaccharide-type structure has the structure (XII):

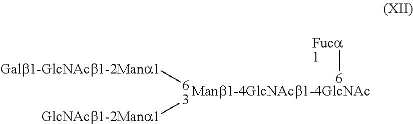

In another embodiment, an NA1F fucosylated biantennary oligosaccharide-type structure has the structure (XIII):

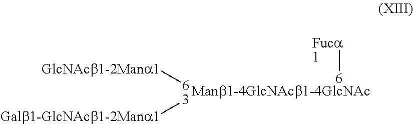

In one embodiment, an NGA2F-GlcNAc, and NA1F-GlcNAc fucosylated biantennary oligosaccharide-type structure has the structure (XIV):

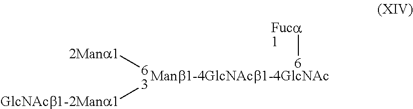

In one embodiment, an NA1F-GlcNAc fucosylated biantennary oligosaccharide-type structure has the structure (XV):

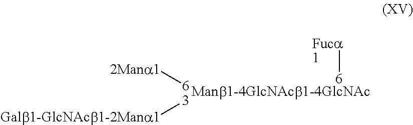

In one embodiment, the fucosylated biantennary oligosaccharide-type structure is independently selected from the group consisting of NGA2F, NA1F, NA2F, NGA2F-GlcNAc, and NA1F-GlcNAc.

As described in the appended examples, a relationship between the level and type of glycoforms of a human antibody in an antibody composition and the rate of serum clearance of the antibody have been discovered. Accordingly, the invention provides compositions of antibodies, or antigen-binding fragments thereof, (e.g., human antibodies, or antigen-binding fragments thereof) comprising varied levels of antibodies, or antigen binding fragments thereof, glycosylated at N-linked glycosylation sites on the Fc region and methods of using these compositions.

The term “level” with respect to an antibody, or antigen-binding fragment thereof, which is glycosylated at an N-linked glycosylation site on the Fc region in a composition refers to the relation of one glycoform in the composition to the whole of the glycoform levels in the composition and is expressed as a percentage of the whole, e.g., 0-100%. The level in a composition may be an absolute amount as measured in molecules, moles, or weight percent.

Compositions comprising varying levels of glycoforms of a human antibody, or antigen-binding fragment thereof, are useful in that by varying the glycoform compositions a desired rate of serum clearance may be achieved. Achieving a desired rate of serum clearance is useful in various clinical indications. For example, if an antibody therapy is administered to treat a chronic condition, such as psoriasis, a long half life and associated slow rate of serum clearance may be desired, for example, so that treatments can be administered less frequently and the patient does not have to make frequent trips to a medical provider for administration of the therapy. Alternatively, when an antibody therapy is administered to treat an acute condition, such as sepsis, a short half life and associated rapid rate of serum clearance may be desired, for example, so that the potential for any adverse effects may be lessened.

As used herein, the term “desired rate of serum clearance” refers to a rate of serum clearance of a composition comprising varying levels of glycoforms of an antibody, or antigen-biding fragment thereof, appropriate for the treatment of a medical condition for which the antibody or composition is being administered.

Furthermore, as described in the appended examples, simulations of bioequivalence studies demonstrated that increasing the level of oligomannose-type structures in the antibody composition to more than about 30%, e.g., about 31-100%, increases the rate of serum clearance of the antibody, or antigen-binding fragment thereof. Similarly, decreasing the level of oligomannose-type structures to less than about 30%, e.g., about 10-30%, decreases the rate of serum clearance of the antibody, or antigen-binding fragment thereof.

Modulating the level of oligomannose-like structures and/or modulating the level of fucosylated bianntenary-type structures in the composition may be used to “modulate” (e.g., increase or decrease) the rate of serum clearance. As used herein, a “rapid rate of serum clearance” is art known and includes the rate of clearance of a human antibody composition as described herein which comprises two types of oligosaccharide-type structures in which the level of oligomannose-type structures is greater than about 30% or greater than about 50% of the total level of glycosylated antibodies, or antigen-binding fragments thereof, in the composition. A “slow rate of serum clearance” is art known and includes the rate of clearance of a human antibody composition which comprises two types of oligosaccharide-type structures in which the level of oligomannose-type structures is about 0-100% or about 10-30% of the total level of glycosylated antibodies, or antigen-binding fragments thereof, in the composition.

The rate of serum clearance of an antibody, or antigen-binding fragment thereof, may be determined by methods routine to one of ordinary skill in the art and as described herein.

A modulation (e.g., increase or decrease) in the rate of serum clearance of a composition comprising a human antibody, or antigen-binding fragment thereof, may be determined by, for example, comparing the rate of serum clearance of the composition with an appropriate control. The choice of an appropriate control is routine to one of ordinary skill in the art. For example, the rate of serum clearance of a composition comprising a human antibody, or antigen-binding fragment thereof, may be determined by comparing the rate of serum clearance of the compostion with the rate of serum clearance of a second composition consisting essentially of the same components but for a varied N-glycan, e.g., a varied level and/or type of N-glycan. An appropriate control may also be a composition comprising the antibody, or antigen-binding fragment thereof, produced recomninantly in a different cell type. For example, a first composition may be produced in CHO cells, and a control composition may be produced in a different type of cells.

The term “pharmacokinetics” refers to how the body interacts with a therapeutic product, such as an antibody, after its administration. Pharmacokinetic parameters describe the extent and rate of absorption, distribution, metabolism, and excretion.

The term “serum clearance” refers to the volume of serum cleared of the antibody, or antigen-binding fragment thereof, per unit time. Serum clearance (Cl) is defined as follows:

Cl=V_{d}×Kₑ=D/AUC.

V_{d} is the apparent volume in which the antibody is distributed immediately after it has been administered and has equilibrated between serum and the surrounding tissues. Kₑ is the rate at which the antibody is removed from the body. D is the dose of the antibody. AUC is the area under the curve, or the integral of the serum antibody concentration (Cₚ) after it is administered. V_{d} is further defined as follows:

V_{d}=D/C₀.

where C₀ is the initial or steady-state concentration of the antibody in serum. Kₑ is defined as

Kₑ=:ln(2)/T_{1/2}=Cl/V_{d}.

where T_{1/2} is the biological half life, or the time required for the concentration of the antibody to reach half of its original value.
AUC is the area under the curve, or the integral of the serum antibody concentration (Cₚ) after it is administered.

Thus, the rate of serum clearance is inversely related to the half life of the antibody. The half life of normal human IgG1, IgG2, and IgG4 is about 20-25 days, and the half life of normal human IgG3 is about 7 days (Jefferis, R. (2009), Trends in Pharmacological Sciences 30(7): 356-362).

The term “antibody” broadly refers to any immunoglobulin (Ig) molecule comprised of four polypeptide chains, two heavy (H) chains and two light (L) chains, interconnected by disulfide bonds or any functional fragment, mutant, variant, or derivation thereof, which retains the essential epitope binding features of an Ig molecule. Such mutant, variant, or derivative antibody formats are known in the art, nonlimiting embodiments of which are discussed herein. Immunoglobulin molecules can be of any type (e.g., IgG, IgE, IgM, IgD, IgA and IgY), class (e.g., IgG1, IgG2, IgG 3, IgG4, IgA1 and IgA2) or subclass.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Light chains are classified as either kappa or lambda. Heavy chains are classified as gamma, mu, alpha, delta, or epsilon, and define the antibody's isotype as IgG, IgM, IgA, IgD and IgE, respectively.

An immunoglobulin constant domain refers to a heavy or light chain constant domain. Human IgG heavy chain and light chain constant domain amino acid sequences are known in the art.

The term “Fc region” refers to the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Specifically, in IgG, IgA and IgD types, the Fc region is composed of two identical protein fragments derived from CH2 and CH3 of the heavy chains. Fc regions of IgM and IgE contain three heavy chain constant domains, CH2, CH3, and CH4.

Replacements of amino acid residues in the Fc portion to alter antibody effector function are known in the art (U.S. Pat. Nos. 5,648,260 and 5,624,821). The Fc portion of an antibody mediates several important effector functions, e.g., cytokine induction, antibody dependent cell mediated cytotoxicity (ADCC), phagocytosis, complement dependent cytotoxicity (CDC) and half-life/clearance rate of antibody and antigen-antibody complexes. Certain human IgG isotypes, particularly IgG1 and IgG3, mediate ADCC and CDC via binding to FcγRs and complement C1q, respectively.

As used herein, the term “Fc region” includes also naturally occurring allelic variants of the Fc region of an immunoglobulin (antibody) as well as variants having alterations which are substitutions, additions, or deletions but which do not affect Ans297 glycosylation. For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U., et al., Science 247 (1990) 1306-1310).

The CH2 domain of each heavy chain contains a single site for N-linked glycosylation at an asparagine residue linking an N-glycan to the immunoglobulins molecule at “asparagine residue 297” (“Asn-297”) (Kabat et al., Sequences of proteins of immunological interest, Fifth Ed., U.S. Department of Health and Human Services, NIH Publication No. 91-3242).

The term “lambda (λ) light chain” refers to a small polypeptide unit of an antibody that is encoded by the immunoglobulin lambda locus on chromosome 22. As indicated above, in mammals, there are two types of antibody light chains, the lambda (λ) light chain and the kappa (κ) chain. As used here, the term λ light chain includes mutant, variant, or derivative formats of the λ light chain.

The term “antigen-binding portion” or “antigen-binding fragment” of an antibody (or simply “antibody portion”) refers to one or more fragments of an antibody that retain the ability to specifically bind to an antigen (e.g., hIL-12). Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al. (1989) Nature 341:544-546, Winter et al., PCT publication WO 90/05144 A1), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426 and Huston et al. (1988) Proc. Natl. Acad. Sci. USA 85:5879-5883). Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. USA 90:6444-6448; Poljak et al. (1994) Structure 2:1121-1123). Such single chain antibodies are also intended to be encompassed within the term “antigen-binding portion” of an antibody as is well known in the art (Kontermann and Dubel eds., Antibody Engineering (2001) Springer-Verlag. New York, 790 (ISBN 3-540-41354-5).

Still further, an antibody or antigen-binding portion thereof may be part of a larger immunoadhesion molecules, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Preferred antigen binding portions are complete domains or pairs of complete domains.

The term “multivalent binding protein” refers to a binding protein comprising two or more antigen binding sites. In an embodiment, the multivalent binding protein is engineered to have three or more antigen binding sites, and is generally not a naturally occurring antibody. The term “multispecific binding protein” also refers to a binding protein capable of binding two or more related or unrelated targets. Dual variable domain (DVD-Ig™) binding proteins comprise two or more antigen binding sites and are tetravalent or multivalent binding proteins. DVD-Ig™s may be monospecific, i.e., capable of binding one antigen, or multispecific, i.e., capable of binding two or more antigens. DVD-Ig™ binding proteins comprising two heavy chain DVD-Ig™ polypeptides and two light chain DVD-Ig™ polypeptides are referred to as DVD-Ig™. Each half of a DVD-Ig™ comprises a heavy chain DVD-Ig™ polypeptide, and a light chain DVD-Ig™ polypeptide, and two antigen binding sites. Each binding site comprises a heavy chain variable domain and a light chain variable domain with a total of 6 CDRs involved in antigen binding per antigen binding site.

The term “bispecific antibody” refers to full-length antibodies that are generated by quadroma technology (Milstein, C. and A. C. Cuello (1983) Nature 305(5934):537-40), by chemical conjugation of two different monoclonal antibodies (Staerz, U. D. et al. (1985) Nature 314(6012):628-31), or by knob-into-hole or similar approaches that introduce mutations in the Fc region (Holliger, P. et al. (1993) Proc. Natl. Acad. Sci. USA 90:6444-8.18), resulting in multiple different immunoglobulin species of which only one is the functional bispecific antibody. By molecular function, a bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen to which it binds.

The term “dual-specific antibody” refers to a full-length antibody that can bind two different antigens (or epitopes) in each of its two binding arms (a pair of HC/LC) (PCT Publication No. WO 02/02773). Accordingly, a dual-specific binding protein has two identical antigen binding arms, with identical specificity and identical CDR sequences, and is bivalent for each antigen to which it binds.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method. In an embodiment, the monoclonal antibody is produced by hybridoma technology.

The term “chimeric antibody” refers to an antibody that comprises heavy and light chain variable region sequences from one species and constant region sequences from another species, such as antibodies having murine heavy and light chain variable regions linked to human constant regions.

The term “CDR-grafted antibody” refers to an antibody that comprises heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

The term “human antibody” includes antibodies having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations preferably are introduced using the “selective mutagenesis approach” described in U.S. Pat. No. 6,914,128, the entire contents of which are incorporated by reference herein. The human antibody can have at least one position replaced with an amino acid residue, e.g., an activity enhancing amino acid residue which is not encoded by the human germline immunoglobulin sequence. The human antibody can have up to twenty positions replaced with amino acid residues that are not part of the human germline immunoglobulin sequence. In other embodiments, up to ten, up to five, up to three or up to two positions are replaced. In a preferred embodiment, these replacements are within the CDR regions as described in detail below. However, the term “human antibody”, as used herein, is not intended to include antibodies in which CDR sequences derived from the germline of another mammalian species, such as a mouse, have been grafted onto human framework sequences. Methods for generation human or fully human antibodies are known in the art and include EBV transformation of human B cells, selection of human or fully human antibodies from antibody libraries prepared by phage display, yeast display, mRNA display or other display technologies, and also from mice or other species that are transgenic for all or part of the human Ig locus comprising all or part of the heavy and light chain genomic regions defined further above. Selected human antibodies may be affinity matured by art recognized methods including in vitro mutagenesis, preferably of CDR regions or adjacent residues, to enhance affinity for the intended target.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library; antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or backmutation or both.

An “isolated antibody”, as used herein, is intended to refer to an antibody that is substantially free of other antibodies having different antigenic specificities (e.g., an isolated antibody that specifically binds human IL-12 and/or IL-23, e.g., binds the p40 subunit of human IL-12/1L-23, is substantially free of antibodies that specifically bind antigens other than human IL-12 and IL-23). An isolated antibody that specifically binds human IL-12 and/or IL-23 may, however, have cross-reactivity to other antigens, such as human IL-12 and/or IL-23 molecules from other species. Moreover, an isolated antibody may be substantially free of other cellular material and/or chemicals.

A “neutralizing antibody”, as used herein (or an “antibody that neutralizes human IL-12 and/or IL-23 activity” or an “antibody that neutralizes the activity of the p40 subunit of IL-12/IL-23”), is intended to refer to an antibody whose binding to human IL-12 and/or IL-23 (e.g., binding to the p40 subunit of IL-12/IL-23) results in inhibition of the biological activity of human IL-12 and/or IL-23 (e.g., biological activity of the p40 subunit of IL-12/IL-23). This inhibition of the biological activity of human IL-12 and/or IL-23 can be assessed by measuring one or more indicators of human IL-12 and/or IL-23 biological activity, such as inhibition of human phytohemagglutinin blast proliferation in a phytohemagglutinin blast proliferation assay (PHA), or inhibition of receptor binding in a human IL-12 and/or IL-23 receptor binding assay (e.g., an interferon-gamma induction Assay). These indicators of human IL-12 and/or IL-23 biological activity can be assessed by one or more of several standard in vitro or in vivo assays known in the art, and described in U.S. Pat. No. 6,914,128 (e.g., Example 3 at column 109, line 31 through column 113, line 55), the entire contents of which are incorporated by reference herein.

The term “humanized antibody” refers to an antibody that comprises heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. Also a “humanized antibody” is an antibody or a variant, derivative, analog or fragment thereof that specifically binds to an antigen of interest and which comprises a framework (FR) region having substantially the amino acid sequence of a human antibody and a complementary determining region (CDR) having substantially the amino acid sequence of a non-human antibody.

The phrase “human interleukin 12” or “human IL-12” (abbreviated herein as hIL-12, or IL-12), as used herein, includes a human cytokine that is secreted primarily by macrophages and dendritic cells. The term includes a heterodimeric protein comprising a 35 kD subunit (p35) and a 40 kD subunit (p40) which are both linked together with a disulfide bridge. The heterodimeric protein is referred to as a “p70 subunit”. The structure of human IL-12 is described further in, for example, Kobayashi, et al. (1989) J. Exp Med. 170:827-845; Seder, et al. (1993) Proc. Natl. Acad. Sci. 90:10188-10192; Ling, et al. (1995) J. Exp Med. 154:116-127; Podlaski, et al. (1992) Arch. Biochem. Biophys. 294:230-237; and Yoon et al. (2000) EMBO Journal 19(14): 3530-3541. The term human IL-12 is intended to include recombinant human IL-12 (rh IL-12), which can be prepared by standard recombinant expression methods.

The phrase “human interleukin 23” or “human IL-23” (abbreviated herein as hIL-23, or IL-23), as used herein, includes a human cytokine that is secreted primarily by macrophages and dendritic cells. The term includes a heterodimeric protein comprising a 19 kD subunit (p19) and a 40kD subunit (p40) which are both linked together with a disulfide bridge. The heterodimeric protein is referred to as a “p40/p19” heterodimer. The structure of human IL-23 is described further in, for example, Beyer et al. (2008) J. Mol. Biol. 382:942-955; Lupardus et al. (2008) J. Mol. Biol. 382:931-941. The term human IL-23 is intended to include recombinant human IL-23 (rhIL-23), which can be prepared by standard recombinant expression methods.

The phrase “p40 subunit of human IL-12/IL-23” or “p40 subunit of human IL-12 and/or IL-23,” or “p40 subunit” as used herein, is intended to refer to a p40 subunit that is shared by human IL-12 and human IL-23. The structure of the p40 subunit of IL-12/IL-23 is described in, for example, Yoon et al. (2000) EMBO Journal 19(14): 3530-3541.

### II. COMPOSITIONS OF THE INVENTION

The present invention provides compositions comprising an antibody, or antigen-binding fragment thereof, (e.g., a human antibody, or antigen-binding fragment thereof) which exhibit a desired rate of serum clearance. In one aspect, the compositions include a first level of an antibody, or antigen-binding fragment thereof, (e.g., a human antibody, or antigen-binding fragment thereof) which is glycosylated at an N-linked glycosylation site on the Fc region of the antibody with an oligomannose type structure, and a second level of the antibody, or antigen binding portion thereof, which is glycosylated at the N-linked glycosylation site on the Fc region with a fucosylated biantennary oligosaccharide-type structure.

The present invention also provides compositions comprising an antibody, or antigen binding portion thereof, (e.g., a human antibody, or antigen-binding fragment thereof) which include about 0-100% of the antibody, or antigen binding portion thereof, which is glycosylated at an N-linked glycosylation site on the Fc region with an oligomannose-type structure and about 0-100% of the antibody, or antigen binding portion thereof, which is glycosylated at the N-linked glycosylation site on the Fc region with a fucosylated biantennary oligosaccharide-type structure.

The present invention further provides compositions comprising ABT-874, or an antigen binding portion thereof, in which about 0-100% of the ABT-874 is glycosylated at Asn 297 with an oligomannose structure that is independently selected from the group consisting of M5, M6, M7, M8 and M9, and about 0-100% of the ABT-874 is glycosylated at Asn 297 with a fucosylated biantennary oligosaccharide structure that is independently selected from the group consisting of NGA2F, NA1F, NA2F, NGA2F-GlcNAc, and NA1F-GlcNAc.

The N-linked glycosylation site on the Fc region of the antibody, or antigen-binding fragment thereof, may be an asparagine residue or an arginine residue. In one embodiment, the N-linked glycosylation site on the Fc region of the antibody, or antigen-binding fragment thereof, is an asparagine residue. In one embodiment the asparagine residue is Asn 297. It is also contemplated that in addition to glycosylation at Asn297 the antibody, or antigen-binding portion thereof, may be glycosylated at other sites, e.g., N-linked glycosylation sites, on the antibody, or antigen-binding portion thereof.

The oligomannose-type structure of the glycosylated antibody, or antigen-binding fragment thereof, may be M5, M6, M7, M8 and/or M9. In one embodiment, the oligomannose-type structure of the glycosylated antibody, or antigen-binding fragment thereof, is independently selected from the group consisting of M5, M6, M7, M8 and M9.

The level of the oligomannose-type structure of the glycosylated antibody, or antigen-binding fragment thereof, in the composition may be about 0-100% of the total level of the antibody, or antigen-binding portion thereof, that is included in the composition. In one embodiment, the first level (the level of the oligomannose-type structure of the glycosylated antibody, or antigen-binding fragment thereof) in the composition is selected from the group consisting of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 8%, 99%, and about 100%. In another embodiment, the first level of the antibody, or antigen-binding portion thereof, in the composition is selected from the group consisting of about 10%, 11%, 12%, 13%, 14%, 15%, 16%, 17%, 18%, 19%, 20%, 21%, 22%, 23%, 24%, 25%, 26%, 27%, 28%, 29% and about 30%. It is intended that, in some embodiments this first level may have levels of about 0-10%, about 10-20%, about 10-30%, about 20-30%, about 30-40%, about 50-60%, about 60-70%, about 70-80%, about 80-90% or about 90-100%. In other embodiments, this first level may range from about 0-3%, about 4-10%, about 11-15%, about 16-20%, about 21-25%, about 26-30%, about 31-35%, about 36-40%, about 41-45%, about 46-50%, about 51-55%, about 56-60%, about 61-65%, about 66-70%, about 71-75%, about 76-80%, about 81-85%, about 86-90%, about 91-95%, about 96-100%. Levels and ranges intermediate to the above recited levels and ranges, e.g., about 10.5% or 5-33%, are also intended to be part of this invention. For example, ranges of values using a combination of any of the above recited values as upper and/or lower limits are intended to be included.

The fucosylated biantennary oligosaccharide-type structure of the glycosylated antibody, or antigen-binding fragment thereof, may be NGA2F, NA1F, NA2F, NGA2F-GlcNAc, and/or NA1F-GlcNAc. In one embodiment, the fucosylated biantennary oligosaccharide type structure is independently selected from the group consisting of NGA2F, NA1F, NA2F, NGA2F-GlcNAc, and NA1F-GlcNAc.

The level of the fucosylated biantennary oligosaccharide-type structure of the glycosylated antibody, or antigen-binding fragment thereof, in the composition may be about 0-100% of the total level of the antibody, or antigen-binding portion thereof, that is included in the composition. In one embodiment, the second level (the level of the fucosylated biantennary oligosaccharide-type structure of the glycosylated antibody, or antigen-binding fragment thereof) in the composition is selected from the group consisting of about 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 8%, 99%, and about 100%. In another embodiment, the second level of the antibody, or antigen-binding portion thereof, in the composition is selected from the group consisting of about 70%, 71%, 72%, 73%, 74%, 75%, 76%, 77%, 78%, 79%, 80%, 81%, 82%, 83%, 84%, 85%, 86%, 87%, 88%, 89% and about 90%. It is intended that, in other embodiments, this second level may range from about 0-100%, with some embodiments having levels of about 0-10%, about 10-20%, about 20-30%, about 30-40%, about 50-60%, about 60-70%, about 70-80%, about 80-90%, about 70-90%, or about 90-100%. In other embodiments, this first level could range from about 0-5%, about 6-10%, about 11-15%, about 16-20%, about 21-25%, about 26-30%, about 31-35%, about 36-40%, about 41-45%, about 46-50%, about 51-55%, about 56-60%, about 61-65%, about 66-70%, about 71-75%, about 76-80%, about 81-85%, about 86-90%, about 90-96%, or about 97-100%. Levels and ranges intermediate to the above recited levels and ranges, e.g., about 70.5% or about 73-81%, are also intended to be part of this invention. For example, ranges of values using a combination of any of the above recited values as upper and/or lower limits are intended to be included.

The compositions of the invention serve to provide desired rates of serum clearance, e.g., a rapid rate or a slow rate of serum clearance, of the composition. When a rapid rate of serum clearance is desired, the level of the oligomannose-type structure of the glycosylated antibody, or antigen-binding fragment thereof, in the composition may be greater than about 50%. In one embodiment, when a rapid rate of serum clearance is desired, the level of the oligomannose-type structure of the glycosylated antibody, or antigen-binding fragment thereof, in the composition is about 51-100% of the total level of the antibody, or antigen-binding portion thereof, that is included in the composition. When a slow rate of serum clearance is desired, the level of the oligomannose-type structure of the glycosylated antibody, or antigen-binding fragment thereof, in the composition is about 0-100% of the total level of the antibody, or antigen-binding portion thereof, that is included in the composition.

Antibodies suitable for use in the compositions of the invention include polyclonal, monoclonal, recombinant antibodies, single chain antibodies, hybrid antibodies, chimeric antibodies, humanized antibodies, or antigen-binding fragments thereof. Antibody-like molecules containing one or two binding sites for an antigen and a Fc-part of an immunoglobulin can also be used. In one embodiment, antibody, or antigen-binding fragments thereof, suitable for use in the compositions and methods of the invention are human antibodies, or antigen-binding fragments thereof. In one embodiment, a human antibody, or antigen-binding fragment thereof, suitable for use in the compositions and methods of the invention is a recombinantly produced human antibody, or an antigen-binding portion thereof.

In certain embodiments, the antibody comprises a heavy chain constant region, such as IgG1, IgG2, IgG3, IgG4, IgM, IgA and IgE constant regions and any allotypic variant therein as described in Kabat, (Kabat, E. A., et al. (1991) Sequences of Proteins of Immunological Interest, Fifth Edition, U.S. Department of Health and Human Services, NIH Publication No. 91-3242). Preferably, the antibody heavy chain constant region is an IgG1 heavy chain constant region.

The invention includes compositions in which the antibody, or antigen binding portion thereof, is selected from the group consisting of IgG, IgA, IgD, IgE, and IgM.

In another embodiment, the antibody is a lambda chain-containing antibody or antigen binding portion thereof.

In further embodiments, the antibody, or antigen-binding portion thereof, includes an IgG1 Fc region and a λ light chain. The aforementioned IgG1 Fc region and λ light chain may be selected from any of the known human antibodies that contain an IgG1 Fc region and a λ light chain.

Examples of lambda chain-containing antibodies, e.g., lambda chain-containing antibodies that may be included in the compositions and methods of the invention, are well known in the art and are understood to be encompassed by the invention. Examples of lambda chain-containing antibodies include, but are not limited to, the anti-IL-17 antibody Antibody 7 as described in International Application WO 2007/149032 (Cambridge Antibody Technology), the entire contents of which are incorporated by reference herein, the anti-IL-12 antibody J695 (Abbott Laboratories), the anti-IL-13 antibody CAT-354 (Cambridge Antibody Technology), the anti-human CD4 antibody CE9y4PE (IDEC-151, clenoliximab) (Biogen IDEC/Glaxo Smith Kline), the anti-human CD4 antibody IDEC CE9.1/SB-210396 (keliximab) (Biogen IDEC), the anti-human CD80 antibody IDEC-114 (galiximab) (Biogen IDEC), the anti-Rabies Virus Protein antibody CR4098 (foravirumab), and the anti-human TNF-related apoptosis-inducing ligand receptor 2 (TRAIL-2) antibody HGS-ETR2 (lexatumumab) (Human Genome Sciences, Inc.).

In one embodiment, a lambda chain-containing antibody or antigen binding portion thereof, is selected from the group consisting of tositumomab, WRI-170, WO1, TNF-H9G1, THY-32, THY-29, TEL16, TEL14, Tel 13, SM1, S1-1, RSP4, RH-14, RF-TS7, RF-SJ2, RF-SJ1, RF-AN, PR-TS2, PR-TS1, PR-SJ2, PR-SJ1, PHOX15, PAG-1, 0G-31, NO. 13, NM3E2 SCFV, MUC1-1, MN215, MC116, MAD-2, MAB67, MAB63, MAB60, MAB59, MAB57, MAB56, MAB111, MAB107, L3055-BL, K6H6, K6F5, K5G5, K5C7, K5B8, K4B8, JAC-10, HUC, HMST-1, HIH2, HIH10, HBW4-1, HBP2, HA1, H6-3C4, H210, GP44, GG48, GG3, GAD-2, FOM-A, FOM-1, FOG1-A3, FOG-B, DPC, DPA, DOB1, DO1, CLL001, CLL-249, CD4-74, CB-201, C304 RF, BSA3, B03, BO1, BEN-27, B-33, B-24, ANTI-TEST, ANTI-EST, ANTI-DIGB, ANTI-DIGA, AIG, 9604, 448.9G.F1, 33.H11, 32.B9, 24A5, 1B9/F2, 13E10, 123AV16-1, 11-50, and 1.32.

In one aspect of the invention, the compositions contain a human antibody that binds to an epitope of the p40 subunit of IL-12/IL-23. In one embodiment, the antibody binds to the p40 subunit when the p40 subunit is bound to the p35 subunit of IL-12. In one embodiment, the antibody binds to the p40 subunit when the p40 subunit is bound to the p19 subunit of IL-23. In one embodiment, the antibody binds to the p40 subunit when the subunit is bound to the p35 subunit of 11-12 and when the p40 subunit is bound to the p19 subunit of 11-23. In a preferred embodiment, the antibody, or antigen-binding portion thereof, is an antibody like those described in U.S. Pat. No. 6,914,128, the entire contents of which are incorporated by reference herein. For example, in a preferred embodiment, the antibody binds to an epitope of the p40 subunit of IL-12 to which an antibody selected from the group consisting of Y61 and J695, as described in U.S. Pat. No. 6,914,128, binds. Especially preferred among the human antibodies is ABT-874 as described in U.S. Pat. No. 6,914,128. Other antibodies that bind IL-12 and/or IL-23 and which can be used in the formulations of the invention include the human anti-IL-12 antibody C340, as described in U.S. Pat. No. 6,902,734, the entire contents of which are incorporated by reference herein.

In another embodiment of the invention, the formulation contains a human antibody, or antigen-binding portion thereof, that neutralizes the biological activity of the p40 subunit of human IL-12/IL-23. In one embodiment, the antibody, or antigen-binding portion thereof, neutralizes the biological activity of free p40, e.g., monomer p40 or a p40 homodimer, e.g., a dimer containing two identical p40 subunits. In preferred embodiments, the antibody, or antigen-binding portion thereof, neutralizes the biological activity of the p40 subunit when the p40 subunit is bound to the p35 subunit of 11-12 and/or when the p40 subunit is bound to the p19 subunit of IL-23.

In yet another embodiment of the invention, the formulation contains a human antibody, or antigen-binding portion thereof, which has a heavy chain and light chain CDR3, the amino acid sequences of which are shown in SEQ ID NOs: 25 and 26, respectively. In one embodiment, antibodies suitable for use in the compositions of the invention further comprise a heavy and light chain CDR2, the amino acid sequences of which are shown in SEQ ID NOs: 27 and 28, respectively. In another embodiment, antibodies suitable for use in the compositions of the invention further comprise a heavy and light chain CDR1, the amino acid sequences of which are shown in SEQ ID NOs: 29 and 30, respectively. In yet another embodiment, antibodies suitable for use in the compositions of the invention comprise a heavy chain variable region and a light chain variable region, the amino acid sequences of which are shown in SEQ ID NO: 31 and SEQ ID NO: 32, respectively.

In some embodiments, the present invention provides compositions which include human anti-IL-12 antibodies. Such anti-IL-12 antibodies include, for example, those disclosed in WO0212500A2; U.S. Pat. No. 6,902,734; U.S. Pat. No. 7,063,964; U.S. Pat. No. 7,166,285; U.S. Pat. No. 7,279,157; US2005002937A1; US2008090290A1; EP1309692A2, WO06071804; WO03082206; EP1494712; WO06069036A2; EP1836294A2; US20090202549; US12500120, EP1839120, the entire contents of which are expressly incorporated by reference herein. Additional non-limiting examples of IL-12 antibodies suitable for use in the compositions of the invention are disclosed in U.S. Pat. No. 5,811,523, U.S. Pat. No. 5,457,038, U.S. Pat. No. 5,569,454, U.S. Pat. No. 5,648,072, U.S. Pat. No. 5,648,467, U.S. Pat. No. 6,300,478, U.S. Pat. No. 6,555,658, U.S. Pat. No. 7,122,633, US20020137898, US20040044186, US20070104680, U.S. Pat. No. 6,339,948, U.S. Pat. No. 6,706,264, U.S. Pat. No. 6,830,751, U.S. Pat. No. 7,138,115, US20050079177, US20070020233, U.S. Pat. No. 5,853,697, U.S. Pat. No. 5,780,597, U.S. Pat. No. 6,225,117, US20030204059, U.S. Pat. No. 6,410,824, US20020194631, US20030056233, U.S. Pat. No. 6,902,734, U.S. Pat. No. 7,063,964, U.S. Pat. No. 7,166,285, U.S. Pat. No. 7,279,157, US20030124123, US20050002937, US20050112127, US20050196838, US20050214293, US20080090290, US20030157105, U.S. Pat. No. 7,247,711, US20050137385, U.S. Pat. No. 7,252,971, US20060067936, and US20080038831, the entire contents of which are expressly incorporated by reference herein.

In other embodiments, the present invention provides compositions which include human anti-IL-23 antibodies. Such anti-IL23 antibodies include, for example, those disclosed in WO02097048, US2003157105, WO04101750; U.S. Pat. No. 7,247,711; EP1623011; WO06036745; U.S. Pat. No. 7,252,971; and US2008038831, WO07076524; US2007218064; EP1971366; WO07005955; US2007009526, and EP1896073, the entire contents of which are expressly incorporated by reference herein.

An antibody, or antibody-binding fragment thereof, suitable for use in the compositions and methods of the invention may be prepared by recombinant expression of immunoglobulin light and heavy chain genes in a host cell according to methods routine to one of ordinary skill in the art. To express an antibody recombinantly, a host cell is transfected with one or more recombinant expression vectors carrying DNA fragments encoding the immunoglobulin light and heavy chains of the antibody such that the light and heavy chains are expressed in the host cell and, preferably, secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

To express the antibodies, or antibody portions of the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the light or heavy chain sequences, the expression vector may already carry antibody constant region sequences. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

In order to produce an antibody, or antigen-binding fragment thereof, suitably glycosylated and having a desired rate of serum clearance, animal or plant-based expression systems may be used. For example, Chinese hamster ovary cells (CHO), mouse fibroblast cells and mouse myeloma cells (Arzneimittelforschung. 1998 August; 48(8):870-880), transgenic animals such as goats, sheep, mice and others (Dente Prog. Clin. Biol. 1989 Res. 300:85-98, Ruther et al., 1988 Cell 53(6):847-856; Ware, J., et al. 1993 Thrombosis and Haemostasis 69(6): 1194-1194; Cole, E. S., et al. 1994 J. Cell. Biochem. 265-265), plants (Arabidopsis thaliana, tobacco etc.) (Staub, et al. 2000 Nature Biotechnology 18(3): 333-338) (McGarvey, P. B., et al. 1995 Bio-Technology 13(13): 1484-1487; Bardor, M., et al. 1999 Trends in Plant Science 4(9): 376-380), or insect cells (Spodoptera frugiperda Sf9, Sf21, Trichoplusia ni, etc. in combination with recombinant baculoviruses such as Autographa californica multiple nuclear polyhedrosis virus which infects lepidopteran cells) (Altmans et al., 1999 Glycoconj. J. 16(2):109-123) may be used.

Preferred mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to the antigen, e.g., hIL-12 The molecules expressed from such truncated DNA molecules are also encompassed by the antibodies of the invention. In addition, bifunctional antibodies may be produced in which one heavy and one light chain is specific for one antigen, e.g., IL-12, and the other heavy and light chain are specific for a different antigen, using standard chemical crosslinking methods.

In one embodiment, an antibody, or antigen-binding fragment thereof, suitable for use in the compositions and methods of the invention is prepared using a recombinant expression vector encoding both the antibody heavy chain and the antibody light chain and is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Antibodies or antigen-binding portions thereof, for use in the compositions of the invention can be expressed in an animal (e.g., a mouse) that is transgenic for human immunoglobulin genes (see e.g., Taylor, L. D. et al. (1992) Nucl. Acids Res. 20: 6287-6295). Plant cells can also be modified to create transgenic plants that express the antibody or antigen binding portion thereof, of the invention.

The compositions of the invention may further comprise additional agents. For example, the compositions of the invention may further comprise a buffer, a polyol, and/or a surfactant.

As used herein, “buffer” refers to a buffered solution that resists changes in pH by the action of its acid-base conjugate components. A buffer used in this invention has a pH in the range from about 4.0 to about 4.5, about 4.5 to about 5.0, about 5.0 to about 5.5, about 5.5 to about 6, about 6.0 to about 6.5, about 5.7 to about 6.3, about 6.5 to about 7.0, about 7.5 to about 8.0. Examples of buffers that will control the pH in this range include acetate (e.g. sodium acetate), succinate (such as sodium succinate), gluconate, histidine, citrate (such as sodium citrate), phosphate (e.g., sodium phosphate or potassium phosphate), and other organic acid buffers. In one embodiment, the buffer is selected from the group consisting of L-histidine, sodium succinate, sodium citrate, sodium phosphate, and potassium phosphate. In one embodiment of the invention, the buffer comprises L-histidine. In one embodiment, the buffer of the invention comprises 1-50 mM histidine, with a pH of 5-7. In one embodiment of the invention, the buffer comprises 10 mM histidine with a pH of about 6.

A “polyol” is a substance with multiple hydroxyl groups, and includes sugars (reducing and nonreducing sugars), sugar alcohols and sugar acids. Preferred polyols herein have a molecular weight which is less than about 600 kD (e.g., in the range from about 120 to about 400 kD). A “reducing sugar” is one that contains a hemiacetal group that can reduce metal ions or react covalently with lysine and other amino groups in proteins and a “nonreducing sugar” is one that does not have these properties of a reducing sugar. Examples of reducing sugars are fructose, mannose, maltose, lactose, arabinose, xylose, ribose, rhamnose, galactose and glucose. Nonreducing sugars include sucrose, trehalose, sorbose, melezitose and raffinose. Mannitol, xylitol, erythritol, threitol, sorbitol and glycerol are examples of sugar alcohols. As to sugar acids, these include L-gluconate and metallic salts thereof. Where it desired that the formulation is freeze-thaw stable, the polyol is preferably one that does not crystallize at freezing temperatures (e.g., −20° C.) such that it destabilizes the antibody in the formulation. The polyol may also act as a tonicity agent. In one embodiment, the polyol is selected from the group consisting of mannitol and sorbitol. In one embodiment of the invention, one ingredient of the composition is mannitol in a concentration of about 10 to about 100 mg/ml (e.g., about 1-10%). In a particular embodiment of the invention, the concentration of mannitol is about 30 to about 50 mg/ml (e.g., about 3-5%). In a preferred embodiment of the invention, the concentration of mannitol is about 40 mg/ml (e.g., about 4%).

A “surfactant” is also referred to as a detergent. Exemplary detergents include nonionic detergents such as polysorbates (e.g., polysorbates 20, or 80) or poloxamers (e.g., poloxamer 188). The amount of detergent added is such that it reduces aggregation of the formulated antibody and/or minimizes the formation of particulates in the formulation and/or reduces adsorption. In a preferred embodiment of the invention, the formulation includes a surfactant that is a polysorbate. In another preferred embodiment of the invention, the formulation contains the detergent polysorbate 80 or Tween 80. Tween 80 is a term used to describe polyoxyethylene (20) sorbitanmonooleate (see Fiedler, Lexikon der Hifsstoffe, Editio Cantor Verlag Aulendorf, 4th ed., 1996). In one embodiment, the surfactant is selected from the group consisting of polysorbate 80, polysorbate 20, and BRIJ surfactants. In one preferred embodiment, the composition contains between about 0.001 to about 0.1% polysorbate 80, or between about 0.005 and 0.05% polysorbate 80, for example, about 0.001, about 0.005, about 0.01, about 0.05, or about 0.1% polysorbate 80. In a preferred embodiment, about 0.01% polysorbate 80 is found in the composition of the invention.

In another embodiment, a stabilizer or antioxidant, such as methionine may be added to the compositions. Other stabilizers useful in compositions of the invention are known to those of skill in the art and include, but are not limited to, glycine and arginine.

The compositions, e.g., pharmaceutical compositions, of the invention are suitable for administration to a subject. Typically, the pharmaceutical composition comprises a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

The compositions of the invention, as well as compositions developed using the methods of the invention, can be incorporated into a pharmaceutical composition suitable for parenteral administration. Preferably, the antibody or antibody-portions will be prepared as an injectable solution containing about 0.1-about 250 mg/ml antibody. In certain embodiments, the antibody, or antigen-binding portion thereof, e.g., a human anti-IL-12 antibody, or antigen-binding portion thereof, is present in a solution, e.g., an injectable solution at a concentration of about 40 mg/ml, 50, 60, 70, 80, 90, 100, 110, 120, 130, 140, 150, 160, 170, 180, 190, 200, 210, 22, 230, 240, or about 250 mg/ml.

The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%).

In one embodiment, the composition includes the antibody at a dosage of about 0.01 mg/kg-10 mg/kg. More preferred dosages of the antibody include about 1 mg/kg administered every other week, or about 0.3 mg/kg administered weekly.

In general, a suitable dose, e.g., daily dose, of a composition of the invention will be that amount of the composition that is the lowest dose effective to produce a therapeutic effect. Such an effective dose will generally depend upon the factors described above. In one embodiment, an effective amount of the compositions of the present invention is an amount that inhibits IL-12 and/or IL-23 activity (e.g., activity of the p40 subunit of IL-12/IL-23) in a subject suffering from a disorder in which IL-12 and/or IL-23 activity is detrimental. In one embodiment; the composition provides an effective dose of 40 mg, 50 mg, 80 mg, or 100 mg per injection of the active ingredient, the antibody. In another embodiment, the composition provides an effective dose which ranges from about 0.1 to 250 mg of antibody. If desired, the effective dose of the composition may be administered as two, three, four, five, six or more sub-doses administered separately at appropriate intervals throughout the day, optionally, in unit dosage forms.

In an embodiment of the invention, the dosage of the antibody in the composition is between about 1 to about 200 mg. In an embodiment, the dosage of the antibody in the composition is between about 30 and about 140 mg, between about 40 and about 120 mg, between about 50 and about 110 mg, between about 60 and about 100 mg, or between about 70 and about 90 mg. In a further embodiment, the composition includes an antibody dosage, or antigen binding fragment thereof, that binds to IL-12 and/or IL-23 (e.g., binds to the p40 subunit of IL-12 and/or IL-23) for example, at about 1, 10, 20, 30, 40, 50, 60, 70, 80, 90, 100, 110, 120, 130, 140, 150, 160, 170, 180, 190, 200, 210, 220, 230, 240 or about 250 mg.

Ranges intermediate to the above recited dosages, e.g., about 2-139 mg, are also intended to be part of this invention. For example, ranges of values using a combination of any of the above recited values as upper and/or lower limits are intended to be included.

It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

The antibodies and antibody-portions of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound of the composition may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978. In certain embodiments, a composition of the invention may be orally administered, for example, with an inert diluent or an assimilable edible carrier. The composition may also be enclosed in a hard or soft shell gelatin capsule, compressed into tablets, or incorporated directly into the subject's diet. For oral therapeutic administration, the composition may be incorporated with excipients and used in the form of ingestible tablets, buccal tablets, troches, capsules, elixirs, suspensions, syrups, wafers, and the like. To administer a composition of the invention by other than parenteral administration, it may be necessary to coat the composition with, or co-administer the composition with, a material to prevent its inactivation.

Additional therapeutic agents can also be incorporated into the compositions of the invention. In certain embodiments, an antibody or antibody portion of the invention is coformulated with and/or coadministered with one or more additional therapeutic agents. Furthermore, it is also intended that the compositions of the invention may comprise two or more additional therapeutic agents. Compositions that combine therapeutic agents may advantageously utilize lower dosages of the administered therapeutic agents, thus avoiding possible toxicities or complications associated with the various monotherapies. It will be appreciated by the skilled practitioner that when the compositions of the invention comprise a combination therapy, a lower dosage of antibody may be desirable than when the antibody alone is administered to a subject (e.g., a synergistic therapeutic effect may be achieved through the use of combination therapy which, in turn, permits use of a lower dose of the antibody to achieve the desired therapuetic effect).

In one embodiment, the compositions of the invention includes a combination of antibodies (two or more), or a “cocktail” of antibodies. It should be understood that the compositions of the invention can be used alone or in combination with an additional agent, e.g., a therapeutic agent, the additional agent being selected by the skilled artisan for its intended purpose. For example, the additional agent can be a therapeutic agent art-recognized as being useful to treat the disease or condition being treated by the antibody of the present invention. The additional agent also can be an agent which imparts a beneficial attribute to the therapeutic composition e.g., an agent which effects the viscosity of the composition.

In one embodiment, a suitable additional therapeutic agent is selected from the group consisting of budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-113 monoclonal antibodies; anti-IL-6 monoclonal antibodies; growth factors; elastase inhibitors; pyridinyl-imidazole compounds; antibodies to or antagonists of other human cytokines or growth factors, for example, TNF (including adalimumab/HUMIRA), LT, IL-1, IL-2, IL-6, IL-7, IL-8, IL-15, IL-16, IL-18, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD90 or their ligands. The antibodies of the invention, or antigen binding portions thereof, may also be combined with agents, such as methotrexate, cyclosporin, FK506, rapamycin, mycophenolate mofetil, leflunomide, NTHEs, for example, ibuprofen, corticosteroids such as prednisolone, phosphodiesterase inhibitors, adenosine agonists, antithrombotic agents, complement inhibitors, adrenergic agents, agents which interfere with signalling by proinflammatory cytokines such as TNFα or IL-1 (e.g. IRAK, NIK, IKK, p38 or MAP kinase inhibitors), IL-1β converting enzyme inhibitors (e.g., V×740), anti-P7s, p-selectin glycoprotein ligand (PSGL), TNFα converting enzyme inhibitors, T-cell signalling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R, soluble IL-13 receptor (sIL-13)) and antiinflammatory cytokines (e.g. IL-4, IL-10, IL-11, IL-13 and TGFβ).

In another embodiment, a suitable additional therapeutic agent is selected from the group consisting of anti-TNF antibodies and antibody fragments thereof, TNFR-Ig constructs, TACE inhibitors, PDE4 inhibitors, corticosteroids, budenoside, dexamethasone, sulfasalazine, 5-aminosalicylic acid, olsalazine, IL-1β converting enzyme inhibitors, IL-1ra, tyrosine kinase inhibitors, 6-mercaptopurines and IL-11.

In yet another embodiment, a suitable additional therapeutic agent is selected from the group consisting of corticosteroids, prednisolone, methylprednisolone, azathioprine, cyclophosphamide, cyclosporine, methotrexate, 4-aminopyridine, tizanidine, interferon-β1a, interferon-β1b, Copolymer 1, hyperbaric oxygen, intravenous immunoglobulin, clabribine, antibodies or agonists of TNF, LT, IL-1, IL-2, IL-6, IL-7, IL-8, IL-15, IL-16, IL-18, EMAP-II, GM-CSF, FGF, PDGF, antibodies to CD2, CD3, CD4, CD8, CD25, CD28, CD30, CD40, CD45, CD69, CD80, CD86, CD90 or their ligands, methotrexate, cyclosporine, FK506, rapamycin, mycophenolate mofetil, leflunomide, NTHEs, ibuprofen, corticosteroids, prednisolone, phosphodiesterase inhibitors, adenosine agonists, antithrombotic agents, complement inhibitors, adrenergic agents, IRAK, NIK, IKK, p38 or MAP kinase inhibitors, IL-1β converting enzyme inhibitors, TACE inhibitors, T-cell signalling inhibitors, kinase inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1RI, sIL-1RII, sIL-6R, sIL-13R, anti-P7s, p-selectin glycoprotein ligand (PSGL), antiinflammatory cytokines, IL-4, IL-10, IL-13 and TGFβ.

### III. METHODS OF THE INVENTION

The present invention also provides methods for modulating the pharmacokinetics of a composition comprising an antibody or antigen binding-fragment thereof, e.g., a human antibody or antigen-binding fragment thereof, in order to achieve a desired rate of serum clearance of the antibody, or antigen-binding fragment thereof. The methods include modulating a first level of the antibody, or antigen-binding fragment thereof, that is glycosylated with an oligomannose-type structure and modulating a second level of the antibody or antigen-binding fragment thereof, that is glycosylated with a fucosylated biantennary oligosaccharide type structure, wherein the modulation of the first and second level results in a desired rate of serum clearance of the antibody.

The present invention also provides methods for modulating the pharmacokinetics of a composition comprising ABT-874, or antigen-binding portion thereof, in order to achieve a desired rate of serum clearance of the antibody, or an antigen-binding fragment thereof. The methods include modulating a first level of ABT-874 that is glycosylated at an N-linked glycosylation site on the Fc region with an oligomannose-type structure that is independently selected from the group consisting of M5, M6, M7, M8 and M9, and modulating a second level ABT-874 that is glycosylated at the N-linked glycosylation site on the Fc region with a fucosylated biantennary oligosaccharide-type structure that is independently selected from the group consisting of NGA2F, NA1F, NA2F, NGA2F-GlcNAc, and NA 1F-GlcNAc, wherein the modulation of the first and second levels results in a desired rate of serum clearance, thereby modulating the pharmacokinetics of a composition comprising ABT-874, or antigen binding portion thereof.

The present invention further provides methods for modulating the pharmacokinetics of an antibody, or antigen binding portion thereof, e.g., a human antibody or antigen-binding fragment thereof, for administration to a subject in need thereof. The method includes glycosylating the antibody, or antigen binding portion thereof, at an N-linked glycosylation site on the Fc region with an oligomannose-type structure, glycosylating the antibody at an N-linked glycosylation site on the Fc region with a fucosylated biantennary oligosaccharide-type structure, and including the appropriate levels of these glycoforms in a composition in order to achieve a desired rate of serum clearance of the antibody, or an antigen-binding fragment thereof.

Methods for modulating the pharmacokinetics of ABT-874, or an antigen binding portion thereof, are also provided by the present invention. The methods include glycosylating ABT-874, or antigen binding portion thereof, at an N-linked glycosylation site on the Fc region with an oligomannose-type structure, glycosylating ABT-874 at the N-linked glycosylation site on the Fc region with a fucosylated biantennary oligosaccharide-type structure, and including the appropriate levels of these glycoforms in a composition in order to achieve a desired rate of serum clearance of ABT-874, or an antigen-binding fragment thereof.

The present invention also provides methods for modulating the pharmacokinetics of ABT-874, or an antigen binding portion thereof, by glycosylating ABT-874, or an antigen binding portion thereof, at Asn 297 with an oligomannose-type structure that is independently selected from the group consisting of M5, M6, M7, M8 and M9; glycosylating ABT-874 at Asn 297 with a fucosylated biantennary oligosaccharide-type structure that is independently selected from the group consisting of NGA2F, NA1F, NA2F, NGA2F-GlcNAc, and NA1F-GlcNAc; and including the appropriate levels of these glycoforms in a composition in order to achieve a desired rate of serum clearance of ABT-874, or an antigen-binding fragment thereof.

Various methods are known in the art for preparing antibody, or antigen-binding fragments thereof, having particular glycosylation patterns (See, e.g., Jefferis, R. (2009), Trends in Pharmacological Sciences 30(7): 356-362; Jefferis (2007) Vaccines & Antibodies 7(9): 1401-1413).

For example, preparation of a recombinant antibody, or antigen-binding fragment thereof, of interest in a suitable host often results in the production of a composition in which one chain of the antibody, or antigen-binding fragment thereof, of interest is about 100% glycosylated at an N-linked glycosylation site on the Fc region with one or more oligomannose-type structures, and the other chain of the antibody, or antigen-binding fragment thereof, of interest is about 100% glycosylated at an N-linked glycosylation site on the Fc region with one or more fucosylated biantennary oligosaccharide-type structures, thereby providing a composition comprising about 50% of an antibody, or antigen-binding fragment thereof, glycosylated at an N-linked glycosylation site on the Fc region with one or more oligomannose-type structures and about 50% of the antibody, or antigen-binding fragment thereof, glycosylated at an N-linked glycosylation site on the Fc region with one or more fucosylated biantennary oligosaccharide-type structures.

An inhibitor of glycoprotein synthesis and/or glycoprotein processing, may be used to produce an antibody, or antigen-binding fragment thereof, having a desired glycosylation pattern. For example, a selective inhibitor of glycoprotein synthesis and/or glycoprotein processing, may be added to a culture comprising an antibody, or antigen-binding fragment thereof, of interest. Such inhibitors are known in the art and include, for example, kifunensine, which is an inhibitor of mannosidase I enzymatic activity. Kifunensine was first isolated from the actinomycete Kitasatosporia kifunense No. 9482 in 1987 (M. Iwami, et al. (9187), J. Antibiot., 40, 612) and is a cyclic oxamide derivative of 1-amino-mannojirimycin. Addition of kifunensine at sufficient concentrations to a culture comprising an antibody, or antigen-binding fragment thereof, of interest prevents the production of fucosylated biantennary oligosaccharide-type structures, thereby resulting in a composition comprising about 100% of an antibody, or antigen-binding fragment thereof, which is glycosylated at an N-linked glycosylation site on the Fc region with one or more oligomannose-type structures and about 0% of an antibody, or antigen-binding fragment thereof, which is glycosylated at an N-linked glycosylation site on the Fc region with one or more fucosylated biantennary oligosaccharide-type structures. Serial dilutions of the kifunensine and addition of the dilutions to a culture comprising an antibody, or antigen-binding fragment thereof, of interest results in the production of compositions comprising about 80-100% of an antibody, or antigen-binding fragment thereof, which is glycosylated at an N-linked glycosylation site on the Fc region with one or more oligomannose-type structures and about 0-20% of an antibody, or antigen-binding fragment thereof, which is glycosylated at an N-linked glycosylation site on the Fc region with one or more fucosylated biantennary oligosaccharide-type structures.

In order to prepare a composition comprising an antibody, or antigen-binding fragment thereof, of interest which comprises about 100% fucosylated biantennary oligosaccharide-type structures, a composition comprising the antibody, or antigen-binding fragment thereof, may be passed over a Concavalin A column which specifically binds to oligomannose-type structures. If, for example, a buffer such as Tris is used to elute the column, the eluant will be a composition comprising an antibody, or antigen-binding fragment thereof, which is about 0% glycosylated at an N-linked glycosylation site on the Fc region with one or more oligomannose-type structures. If, for example, a buffer comprising, for example, oligomannose or mannose is used to elute the column, the eluant will comprise an antibody, or antigen-binding fragment thereof, which is about 50% glycosylated at an N-linked glycosylation site on the Fc region with one or more oligomannose-type structures. One of ordinary skill in the art can readily vary the concentration of oligomannose and/or mannose in the buffer and/or the collection of various fractions eluted from such a column to prepare compositions comprising an antibody, or antigen-binding fragment thereof, which is between about 0% and about 50% glycosylated at an N-linked glycosylation site on the Fc region with one or more oligomannose-type structures. One of ordinary skill in the art may also readily mix varying amounts of the compositions prepared as described above to arrive at compositions comprising an antibody, or antigen-binding fragment thereof, which is between about 0% and about 100% glycosylated at an N-linked glycosylation site on the Fc region with one or more oligomannose-type structures and/or compositions comprising an antibody, or antigen-binding fragment thereof, which is between about 0% and about 100% glycosylated at an N-linked glycosylation site on the Fc region with one or more fucosylated biantennary oligosaccharide-type structures.

Animal or plant-based expression systems, such as Chinese hamster ovary cells (CHO), mouse fibroblast cells and mouse myeloma cells (Arzneimittelforschung. 1998 August; 48(8):870-880; U.S. Pat. No. 5,545,504); transgenic animals such as goats, sheep, mice and others (Dente Prog. Clin. Biol. 1989 Res. 300:85-98, Ruther et al., 1988 Cell 53(6):847-856; Ware, J., et al. 1993 Thrombosis and Haemostasis 69(6): 1194-1194; Cole, E. S., et al. 1994 J. Cell. Biochem. 265-265); plants (Arabidopsis thaliana, tobacco etc.) (Staub, et al. 2000 Nature Biotechnology 18(3): 333-338) (McGarvey, P. B., et al. 1995 Bio-Technology 13(13): 1484-1487; Bardor, M., et al. 1999 Trends in Plant Science 4(9): 376-380); and insect cells (Spodoptera frugiperda Sf9, Sf21, Trichoplusia ni, etc. in combination with recombinant baculoviruses such as Autographa californica multiple nuclear polyhedrosis virus which infects lepidopteran cells) (Altmans et al., 1999 Glycoconj. J. 16(2):109-123) may also be used to produce an antibody, or antigen-binding fragment thereof, which is glycosylated at an N-linked glycosylation site on the Fc region with one or more oligosaccharide-type structures of interest. Further suitable expression host systems known in the art for production of glycoproteins include: CHO cells: Raju WO9922764A1 and Presta WO03/035835A1; hybridroma cells: Trebak et al., 1999, J. Immunol. Methods, 230: 59-70; insect cells: Hsu et al., 1997, JBC, 272:9062-970, and plant cells: Gerngross et al., WO04/074499A2.

In addition, methods are known in the art for genetically engineering mammalian host cells to increasing the extent of terminal sialic acid in glycoproteins expressed in the cells, to conjugate sialic acid to the protein of interest in vitro prior to administration using a sialic acid transferase and an appropriate substrate, and methods to alter growth medium composition or the expression of enzymes involved in human glycosylation (S. Weikert, et al., Nature Biotechnology, 1999, 17, 1116-1121; Werner, Noe, et al 1998 Arzneimittelforschung 48(8):870-880; Weikert, Papac et al., 1999; Andersen and Goochee 1994 Cur. Opin. Biotechnol. 5: 546-549; Yang and Butler 2000 Biotechnol.Bioengin. 68(4): 370-380). Alternatively cultured human cells may be used.

Microorganisms having genetically altered glycosylation pathways may also be used to produce an antibody, or antigen-binding fragment thereof, which is glycosylated at an N-linked glycosylation site on the Fc region with one or more oligosaccharide-type structures of interest. For example, several glycosyltransferases have been separately cloned and expressed in S. cerevisiae (GalT, GnT I), Aspergillus nidulans (GnT I) and other fungi (Yoshida et al., 1999, Kalsner et al., 1995 Glycoconj. J. 12(3):360-370, Schwientek et al., 1995; Graham and Emr, 1991 J. Cell. Biol. 114(2):207-218; Yoko-o et al. 2001 FEBS Lett. 489(1): 75-80; Shindo et al,. 1993 J. Biol. Chem. 268(35):26338-26345; Chiba et al., 1998 J. Biol. Chem. 273, 26298-26304; Japanese Patent Application Public No. 8-336387; Martinet et al. (Biotechnol. Lett. 1998, 20(12), 1171-1177); U.S. Pat. No. 5,834,251).

Methods and micoroorganisms for producing an antibody, or antigen-binding fragment thereof, which is glycosylated at an N-linked glycosylation site on the Fc region having reduced fucosylation are also known in the art and may be used to produce an antibody, or antigen-binding fragment thereof, which is glycosylated at an N-linked glycosylation site on the Fc region with one or more oligosaccharide-type structures of interest. See, e.g., U.S. Pat. Nos. 6,946,292, 7,214,775, 6,602,684, ,272,066; 6,946,292, 6,803,225, U.S. patent Publication Nos: 2004/0191256, 2004/0136986, 2007/0020260; 2007/0020260, 20040038381, and PCT Publication No. WO/0114522, the entire contents of which are incorporated herein by reference.

In one embodiment of the invention, an antibody, or antigen-binding fragment thereof, which is glycosylated at an N-linked glycosylation site on the Fc region with one or more oligosaccharide-type structures of interest is produced recombinantly in a unicellular or multicellular fungi such as Pichia pastoris, Hansenulapolymorpha, Pichia stiptis, Pichia methanolica, Pichia sp., Kluyveromyces sp., Candida albicans, Aspergillus nidulans, and Trichoderma reseei, as described in U.S. Pat. Nos. 7,629,163, 7,598,055, U.S. Patent Publication No.: 2009/0304690, PCT Publication Nos.: WO 02/00879, WO 03/0569 14, WO 04/074498, WO 04/074499, Choi et al., 2003, PNAS, 100: 5022-5027; Hamilton et al., 2003, Nature, 301: 1244-1246 and Bobrowicz et al., 2004, Glycobiology, 14: 757-766), the entire contents of all of which are incorporated herein by reference.

Once an antibody, or antigen-binding fragment thereof, which is glycosylated at an N-linked glycosylation site on the Fc region with one or more oligosaccharide-type structures of interest is produced recombinantly, it may be purified and isolated using methods known in the art and described in, for example, Kohier & Milstein, (1975) Nature 256:495; Brodeur et al., Monoclonal Antibody Production Techniques and Applications, pp. 51-63, Marcel Dekker, Inc., New York, 1987);. Goding, Monoclonal Antibodies: Principles and Practice, pp. 59-104 (Academic Press, 1986); and Jakobovits et al. (1993) Proc. Natl. Acad. Sci. USA 90:2551-255 and Jakobovits et al, (1993) Nature 362:255-258. Glycan analysis and distribution on the recombinantly produced antibody, or antigen-binding fragment thereof, which is glycosylated at an N-linked glycosylation site on the Fc region with one or more oligosaccharide-type structures of interest may be determined by several mass spectroscopy methods known to one skilled in the art, including but not limited to: HPLC, NMR, LCMS and MALDI-TOF MS. Furthermore, existing methods in the art allow analytical characterization of protein glycoforms to analyze and verify antibody oligosaccharide-type structures. (See, e.g., Beck et al. (2008) Current Pharmaceutical Biotechnology 9: 482-501). These methods include liquid chromatography, electrophoreses and mass-spectrometry, and fingerprinting and structural analysis of peptides, glycopeptides and glycans.

It will be readily apparent to those skilled in the art that other suitable modifications and adaptations of the methods of the invention described herein are obvious and may be made using suitable equivalents without departing from the scope of the invention or the embodiments disclosed herein. Having now described the present invention in detail, the same will be more clearly understood by reference to the following examples, which are included for purposes of illustration only and are not intended to be limiting of the invention. The contents of all figures and all references, patents and published patent applications cited throughout this application, as well as the Figures, are expressly incorporated herein by reference in their entirety.

### EXAMPLES

### Example 1

### Population Pharmacokinetic Analysis of ABT-874 Glycoforms in Healthy Subjects

The pharmacokinetics of ABT-874 were examined following IV, SC, and IM injection in healthy volunteers in four Phase 1 studies. In healthy volunteers, single dose ABT-874 pharmacokinetics were estimated following IV administration, over the 0.1 mg/kg to ˜10 mg/kg (˜700 mg) dose range, and following SC administration over the 0.1 mg/kg to 5.0 mg/kg dose range. Following IV administration, the pharmacokinetics are best described by a two compartment model. The mean terminal half-life was approximately 8 to 9 days following single IV doses of 1.0 to 5.0 mg/kg, and approximately 13 days following a single 700 mg infusion. Following single dose SC administration of 100 mg ABT-874, the median time to peak concentrations were achieved at 60 hours, with a range of 36 to 144 hours, the mean absolute bioavailability was approximately 47.0%, and the mean terminal elimination half-life was approximately 8 days. Following SC administration of doses ranging from 0.1 mg/kg to 5.0 mg/kg, AUC and Cₘₐₓ were dose linear. Following IM administration the absolute bioavailability of ABT-874 was approximately 63%.

ABT-874 has been administered in clinical studies as two formulations, lyophilized powder and liquid formulations, which were manufactured at three different production scales, 1000 L, 3000 L, and 6000 L. Differences in the production lots include varying levels of charge variants, aggregates, and N-linked glycosylation (glycoforms).

As is typical of recombinant monoclonal antibodies, ABT-874 is subject to post-translational modification. Post-translational modifications observed in ABT-874 include N-linked glycosylation at a single site on the Fc region (Asn297) on the heavy chain. No O-linked glycosylation is observed. The predominant carbohydrate species observed in ABT-874 are N-linked fucosylated biantennary oligosaccharide (FBO) structures containing zero and one terminal galactose residues (NGA2F and NA1F, respectively) and are typical of IgG antibodies produced in Chinese hamster ovary (CHO) cells. Abbreviations for the oligosaccharides are summarized in Table 1.

TABLE 1
Abbreviations Used for Oligosaccharides

| Abbreviation | Oligosaccharide | Alternate Name |
|---|---|---|
| NGA2F | Asialo-agalacto biantennary core- | Gal-0 |
| | substituted fucose | |
| NA1F | Asialo-monogalactosylated biantennary | Gal-1 |
| | core-substituted fucose | |
| NA2F | Asialo-bigalactosylated biantennary core- | Gal-2 |
| | substituted fucose | |
| GlcNAc | N-acetylglucosamine | — |
| M, Mann | Mannose | — |

The most prevalent glycoforms observed here for ABT-874 were NGA2F and NA1F. The glycoforms observed within batches for clinical studies ranged from 4 to 10% for oligomannoses.

### Materials and Methods

### Data Sources

Glycoform analysis was conducted using the individual ABT-874 serum concentration-time data collected following a 700 mg IV infusion of the lyophilized powder formulation of ABT-874 manufactured using the 3000 L process. This was Regimen E of Study M10-220.

Study M10-220 was a single-dose, open-label study conducted according to a sequential design. Adult male and female volunteers (N=75) in general good health were selected to participate in the study according to the selection criteria. Fifteen (15) of the 75 subjects enrolled in Study M10-220 received Regimen E, which consisted of a single 700 mg IV infusion administered over 30 minute period on Study Day 1. The ABT-874 formulation used for Regimen E was the reconstituted lyophilized powder manufactured using the 3000 L process.

Following the 700 mg IV infusion of ABT-874 (Regimen E), blood samples for determination of serum ABT-874 concentrations were collected prior to dosing (0 hour), at 30 minutes (end of the 30 minute IV infusion), and at 6, 12, 24, 36, 48, 72, 120, 168, 240, 336, 504, 672, 1008 and 1344 hours after the start of the infusion. Blood samples for determination of ABT-874 glycoform concentrations in human serum were collected at prior to dosing (0 hour), at 30 minutes (end of the 30 minute IV infusion), and at 6, 12, 24, 36, 48, 72, 120, 168, 240, 336, 504 and 672 hours after dosing.

The lyophilized powder for reconstitution manufactured with the 3000 L process was used for the 700 mg IV infusion arm. The percentages and calculated doses of each of the eight ABT-874 glycoforms, as determined by the assay used for analyses of ABT-874 glycoforms in human serum, are shown in Table 2.

TABLE 2
Percentages of ABT-874 Glycoforms in the Lyophilized Powder
Formulation Manufactured Using the 3000 L Process

| Glycoform | Percentage (%) | Dose (mg) |
|---|---|---|
| Fucosylated Biantennary | | |
| NGA2F | 53.11 | 372 |
| NA1F Total | 28.53 | 200 |
| NA2F | 4.17 | 29 |
| NGA2F-GlcNAc | 2.84 | 20 |
| NA1F-GlcNAc | 2.05 | 14 |
| Oligomannoses | | |
| M5 | 5.70 | 40 |
| M6 | 2.29 | 16 |
| M7 | 1.31 | 9 |
| Total ABT-874 | 100 | 700 |

### Measurement Methods for Total ABT-874

Analysis of samples for ABT-874 concentrations in serum was performed using a validated bridging electrochemiluminescent (ECL) assay method at ALTA Analytical Laboratory, San Diego, Calif. The lower limit of quantitation (LLOQ) for ABT-874 was established at 1.5 ng/mL using 1:5 dilution (7.5 ng/mL in undiluted serum).

### Individual ABT-874 Glycoform Analysis

Serum ABT-874 glycoform analysis was performed at the Abbott Bioresearch Center, 100 Research Drive, Worcester, Mass. 01605.

### Measurement Methods for ABT-874 Glycoforms

Eight glycoforms, M5, M6, M7, NAF1 Total, NAF1 GlcNac, NA2F, NGA2F, NGA2F GlcNac, were identified and analyses conducted to assess their percentages of total ABT-874 in human serum.

The percentages of each glycoform were determined using qualified methods for recovery of ABT-874 from human serum using IL 12 affinity chromatography, and for oligosaccharide (glycoform) analysis using 2 aminobenzamide (2 AB) labeling with normal phase high performance liquid chromatography (NPHPLC). The limit of quantitation (LOQ) for the assay was set at 15 μg/mL of ABT-874.

### Population Pharmacokinetics, Data Sets and Analysis Conventions

Final drug substance specifications for glycoforms involve the grouping of the oligosaccharide species based on their structural composition. For these specifications, individual glycoform content are not reported, but the results of each oligosaccharide species are reported. For ABT-874 the specification results are reported based on the presence (FBO) or absence (oligomannose species) of core fucose. Further, during the preliminary pharmacokinetic analyses, within the FBO group, all of the individual FBO species appeared to have similar pharmacokinetic values, as did the mannose species. Therefore, for purposes of the pharmacokinetic analyses, glycoform concentration data were summarized by two groups: Group 1 (Glycoforms NAF1 Total, NAF1 GlcNac, NA2F, NGA2F, NGA2F GlcNac) and Group 2 (Glycoforms M5, M6, M7).

For the purpose of population pharmacokinetic analysis, a NONMEM formatted data file was created from the pharmacokinetic database of Study M10-220. Glycoform percentages were multiplied by total ABT-874 serum concentrations to determine individual ABT-874 glycoform concentrations. The serum concentrations of the individual glycoforms were added for each subject, based on the grouping as defined before.

Serum ABT-874 concentration measurements taken prior to dosing were included in the population based pharmacokinetic analysis. Where available, actual recorded sampling times and dosages were used for analysis instead of protocol times.

### Data for Inclusion in the Pharmacokinetic Analysis

All subjects (N=15) providing at least one serum ABT-874 concentration measurement above the limit of quantification (15 μg/mL) observed after 700 mg ABT-874 dosing IV were included in the analysis.

### Imputation of Data Below Limit of Quantitation

Serum ABT-874 concentration values reported as below the lower limit of quantitation (BLQ) prior to dosing were removed. However, the first serum ABT-874 concentration below the limit of quantitation observed after dosing was set to half of the lower limit of quantitation (LLOQ/2), and all subsequent BLQ values were removed.

### Handling of Outlying Measurements

All individual serum ABT-874 concentration/time data from the clinical database were listed and noted in the dataset if excluded from pharmacokinetic evaluation along with the reason(s) for exclusion.

### Population Pharmacokinetic Modeling

Following single dose IV administration of ABT-874 in IL-001, the pharmacokinetics followed bi-exponential linear disposition. Therefore, the initial assumption was that the pharmacokinetic profile of ABT-874 observed in Study M10 220 followed two compartment linear disposition. If there was strong evidence that another model was more appropriate, modifications to the structural model were to be made.

Population pharmacokinetic models were built using nonlinear mixed effect modeling with the NONMEM software (double precision, version VI level 1.1). The first-order conditional estimation with interaction method (FOCEI) was employed within NONMEM. Models were built in a stepwise manner, increasing in complexity. The likelihood ratio test was used for hypothesis testing to discriminate among alternative hierarchical models. A combination of exponential and/or additive error models were used to characterize the distribution of inter- and intra-subject variability. The appropriateness of various error structures (additive, proportional and combined additive and proportional) were assessed by the fit of the model.

The objective function value (OFV), calculated by the NONMEM software, is approximately Chi-square (χ2) distributed, and the difference in objective function value was used to guide model building. When comparing hierarchical models, an additional model parameter (one degree of freedom [df]) in the pharmacokinetic model was considered to be significant, if it lowered the OFV by more than 6.63 (significance at the 1% level is reached). With two degrees of freedom (two additional model parameter) the critical values was 9.21, respectively. All statistical tests performed were two-tailed and assessed at the 1% significance level.

Selection between non hierarchical models was determined by the Akaike Information Criterion (AIC) (based on the objective function and the number of parameters in a model, lowest AIC value preferred), visual inspection of the fit of the models, the standard errors of the model parameters and the change in inter subject and random residual error.

The influence of covariates (age, sex, race, laboratory measurements) on pharmacokinetics was not investigated, due to the sample size of 15 subjects.

The model at the end of the forward inclusion process was referred to as the full NONMEM model. After the full model was defined, the statistical significance of each influencing factor-parameter relationship (i.e. residual error model) was tested individually in a stepwise deletion method. A particular influencing factor in the full model was fixed to its null value and the model was run to obtain a new objective function. During the stepwise deletion phase, significance of parameters were assessed at the p<0.001 level (increase in OFV by at least 10.83 units for 1 df). This procedure was repeated for all influencing factors until only significant parameters remained. The resulting model was referred to as the final NONMEM model.

The final model consisted of the structural model definition, estimates of population mean and individual fixed effects parameters, and estimates of the inter-individual and residual random effects parameters.

### Model Selection Criteria

The selection of the pharmacokinetic and clinical response models were based on the criteria listed below:

1. The observed and predicted serum concentration from the preferred model were more randomly distributed across the line of unity (a straight line with zero intercept and a slope of one) than alternative models.

2. The weighted residuals of the preferred model showed less systematic bias than the alternative models.

3. The preferred model showed adequate goodness-of-fit plots, and physiologically reasonable and/or statistically significant estimates (95% confidence intervals did not include zero) of mean parameters and their standard errors.

Starting from the a simple model, the complexity of the models was extended until the criteria listed above were met.

### Model Evaluation

The developed models were evaluated both ad hoc, during development, and after the model development was completed. Methods used in model evaluation included goodness-of-fit plots, visual and numeric predictive checks, and bootstrap evaluation.

Model evaluations determined the predictive performance of the developed models and examined the usefulness of the models for describing observations.

### Goodness-of-Fit Plots

Goodness-of-fit plots were generated ad hoc for model evaluation:
Observed versus predicted data plots were presented on linear and logarithmic scales. Population and individual predictions were compared to observations in separate plots, each including the line of unity and a linear or smooth trend line.
Weighted residuals or conditional weighted residuals were plotted versus population predicted values and versus time.
Individual plots were presented showing observations, individual predictions, and population predictions versus time. Clinical response variables were superimposed on the corresponding pharmacokinetic profiles.
Histograms and QQ plots of inter-individual random effect (ETAs) and conditional weighted residuals (CWRES) were presented.
Potential influencing factor-parameter relationships were visualized showing covariates plotted against empirical Bayes estimates (EBE) of relevant parameters and/or random effects.
Scatter plots of the random effect correlation matrix were generated. Selected goodness-of-fit plots for the basic and final models were presented in parallel to demonstrate the improvement in model fit achieved by inclusion of the covariates.

### Visual Predictive Checks

For visual predictive checks, 1000 simulated replicates of the dataset were generated using NONMEM. Subsequently, the simulated predictions were compared to the observed data by superimposing the observed data on selected percentile intervals of the simulated data. Relevant visual predictive checks included plots of observed and predicted concentrations and clinical response versus time. The observations were sorted into time bins using protocol-scheduled times. Observed data were plotted against the corresponding 95% prediction interval derived from the 1000 simulated datasets.

### Bootstrap Evaluation

In order to estimate confidence intervals of the model parameters, 1000 bootstrap replicates were constructed by randomly sampling (with replacement) N subjects from the original dataset, where N was the number of subjects in the original dataset. Model parameters were estimated for each bootstrap replicate and the resulting values were used to estimate medians and confidence intervals.

Bootstrap statistics were based on only replicates that converged successfully. The medians and 95% confidence intervals for bootstrap model parameters were derived as the 50th percentile and the range from the 2.5th to the 97.5th percentiles of the results from individual replicates. Model parameters based on the original dataset were compared against the bootstrap results.

### Clinical Trial Simulations

Clinical trial simulations of bioequivalence studies were performed using Pharsight Trial Simulator® (Version 2.2.1) to simulate the pharmacokinetics of total ABT-874 at the following compositions of glycoform groups (Group 1/Group 2): 100/0, 95/5, 90/10, 80/20, 70/30 and 60/40. The ABT-874 drug product lot used in Study M10 220 consisted of approximately 90% of Group 1 and 10% of Group 2; and was used as the reference product in the simulations. Products at the other Group 1/Group 2 compositions were defined as test products in the simulations.

The final population pharmacokinetic models derived by NONMEM analysis for both glycoform groups were transferred to Pharsight Trial Simulator® using the covariance structure of the point estimates (THETAs) and inter-individual variabilities (ETAs).

For each glycoform composition, serum concentrations of total ABT-874 were simulated for 10,000 subjects per treatment arm. For each subject, maximum serum concentration (Cₘₐₓ) and Area Under the Curve (AUC_{0-28d}) calculated using the trapezoid rule were estimated. One thousand replicates with n=75 subjects per treatment group were randomly drawn from the 10,000 simulated subjects for both test and reference groups. A sample size of 150 subjects (75 per arm) would provide >80% probability of satisfying the equivalence criterion if the true ratio of the Cₘₐₓ and AUC central values (test/reference) is 1.00. The calculation was based on the estimated error term variance using data from the 15 subjects.

Based on the current recommendations on bioequivalence analysis, AUC_{0-28d} and Cₘₐₓ, was log transformed for calculations. Thus 90% confidence interval (CI) of the ratio of test versus reference composition was calculated as:

CI=exp(μ_{T}−μ_{R}±t_{0.05,v}\sqrt{2·MSE/N})

Where μT is the mean of the log AUC_{0-28d} and Cₘₐₓ values in the test arm, μR of the reference arm, t0.05,v is the critical value of t at α=0.05 with v degrees of freedom used for calculation of MSE which was obtained from an ANOVA, N the number of subjects in each arm.

The percentages of replicates where the 90% confidence interval (CI) was outside of the 80% to 125% range (criterion for bioequivalence) were calculated and represented graphically.

### Disposition of Subjects

Adult male and female subjects (N=75) were enrolled in Study M10 220. Fifteen (15) subjects received a single 30 minute 700 mg infusion of ABT-874.

### Demographics

A summary of the demographic data for the subjects included in the population pharmacokinetic analyses can be found in the CSR (R&D/09/065).4

### Data Sets Analyzed

For the population pharmacokinetic analysis, the data from all subjects who were exposed to a single 30 minute 700 mg IV infusion of ABT-874 (N=15) and who had at least one measurable serum concentration were included in the analyses. Two subjects had samples drawn at unscheduled timepoints (Subjects 110 and 111). These samples were not included in the population pharmacokinetic analysis because glycoform concentrations were not determined for these two samples.

### Results

### ABT-874 Glycoform Concentrations

Individual and summary percent glycoform results can be found in Tables 3-10.

TABLE 3
Individual and Summary % Glycoform Results for NGA2F

| | % NGA2F | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 52.01 | 52.77 | 52.63 | 53.57 | 53.77 | 55.36 | 57.74 | 58.11 | 58.76 | 59.29 | — | — | — |
| 102 | 54.52 | 55.01 | 55.57 | 56.20 | 56.20 | 56.55 | 58.06 | 57.17 | 56.70 | 56.45 | — | — | — |
| 103 | 54.88 | 55.37 | 56.10 | 56.06 | 56.98 | 56.88 | 58.82 | 57.99 | 59.29 | — | — | — | — |
| 104 | 53.71 | 55.64 | 54.62 | 55.33 | 55.18 | 56.46 | 57.28 | 56.76 | 59.06 | — | — | — | — |
| 105 | 53.53 | 54.49 | 54.50 | 55.58 | 56.26 | 56.41 | 57.34 | 56.48 | 56.93 | 58.04 | 57.66 | — | — |
| 106 | 52.99 | 54.86 | 53.76 | 54.40 | 56.29 | 56.36 | 56.59 | 55.08 | 54.23 | 53.72 | 53.14 | — | — |
| 107 | 52.09 | 52.05 | 52.92 | 53.04 | 53.17 | 56.14 | 57.29 | 55.98 | 55.85 | 55.03 | 56.45 | 58.50 | 44.26 |
| 108 | 48.86 | 51.67 | 51.44 | 53.35 | 54.28 | 54.67 | 55.07 | 55.52 | 55.36 | 57.67 | 57.41 | — | — |
| 109 | 49.80 | 48.09 | 41.53 | 53.51 | 53.13 | 55.32 | 50.12 | 53.46 | 51.36 | 54.61 | 53.56 | — | — |
| 110 | 50.40 | 51.71 | 52.90 | 55.90 | 56.09 | 55.52 | 56.33 | 57.59 | 58.97 | 56.59 | 55.34 | — | — |
| 111 | 49.77 | 50.54 | 50.41 | 54.89 | 53.81 | 55.54 | 54.17 | 55.94 | 56.44 | 56.16 | 53.38 | — | — |
| 112 | 52.53 | 52.51 | 49.93 | 54.58 | 53.75 | 54.21 | 55.01 | 55.75 | 55.83 | 55.59 | 54.50 | — | — |
| 113 | 52.69 | 51.76 | 52.62 | 53.78 | 54.05 | 54.79 | 55.28 | 57.68 | 56.96 | 57.65 | 55.58 | — | — |
| 114 | 51.82 | 52.10 | 50.82 | 53.05 | 54.29 | 53.24 | 55.22 | 55.73 | 54.98 | 55.84 | 57.47 | — | — |
| 115 | 53.09 | 54.41 | 56.72 | 54.71 | 54.76 | 57.00 | 58.38 | 58.90 | 58.11 | — | — | — | — |
| N | 15 | 15 | 15 | 15 | 15 | 15 | 15 | 15 | 15 | 12 | 10 | 1 | 1 |
| Mean | 52.18 | 52.87 | 52.43 | 54.53 | 54.80 | 55.63 | 56.18 | 56.54 | 56.59 | 56.39 | 55.45 | 58.50 | 44.26 |
| SD | 1.79 | 2.09 | 3.64 | 1.11 | 1.27 | 1.07 | 2.19 | 1.40 | 2.15 | 1.58 | 1.76 | — | — |
| Min | 48.86 | 48.09 | 41.53 | 53.04 | 53.13 | 53.24 | 50.12 | 53.46 | 51.36 | 53.72 | 53.14 | 58.50 | 44.26 |
| Median | 52.53 | 52.51 | 52.90 | 54.58 | 54.29 | 55.54 | 56.59 | 56.48 | 56.70 | 56.31 | 55.46 | 58.50 | 44.26 |
| Max | 54.88 | 55.64 | 56.72 | 56.20 | 56.98 | 57.00 | 58.82 | 58.90 | 59.29 | 59.29 | 57.66 | 58.50 | 44.26 |
| CV % | 3.4 | 4.0 | 6.9 | 2.0 | 2.3 | 1.9 | 3.9 | 2.5 | 3.8 | 2.8 | 3.2 | — | — |

“—” = Not calculated; total ABT-874 serum concentration was below limit of quantitation (LOQ) of 15 μg/mL.

TABLE 4
Individual and Summary % Glycoform Results for NA1F Total

| | % NA1F Total | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 27.76 | 26.85 | 24.25 | 26.38 | 27.71 | 27.37 | 27.05 | 26.18 | 26.12 | 25.30 | — | — | — |
| 102 | 27.76 | 27.06 | 27.50 | 28.21 | 28.03 | 28.08 | 26.02 | 27.49 | 27.70 | 28.87 | — | — | — |
| 103 | 27.25 | 27.39 | 27.74 | 27.71 | 28.14 | 28.06 | 27.45 | 27.23 | 27.98 | — | — | — | — |
| 104 | 27.90 | 26.69 | 26.96 | 26.08 | 27.85 | 26.72 | 25.38 | 25.95 | 25.49 | — | — | — | — |
| 105 | 26.83 | 27.12 | 26.91 | 27.54 | 27.59 | 27.92 | 27.80 | 26.59 | 26.63 | 26.70 | 26.42 | — | — |
| 106 | 24.36 | 25.20 | 25.27 | 24.39 | 24.72 | 24.80 | 25.76 | 24.40 | 24.60 | 26.53 | 25.43 | — | — |
| 107 | 25.12 | 25.44 | 24.53 | 25.50 | 26.54 | 27.98 | 26.98 | 28.54 | 27.22 | 27.66 | 26.95 | 28.58 | 24.03 |
| 108 | 25.50 | 25.49 | 25.65 | 26.25 | 25.63 | 26.27 | 25.70 | 26.20 | 26.28 | 25.18 | 25.50 | — | — |
| 109 | 24.23 | 23.08 | 21.69 | 26.17 | 26.15 | 26.78 | 23.78 | 26.07 | 24.61 | 24.74 | 23.63 | — | — |
| 110 | 25.36 | 26.69 | 25.92 | 26.72 | 27.24 | 26.56 | 25.98 | 25.80 | 25.60 | 24.86 | 23.07 | — | — |
| 111 | 25.12 | 25.27 | 24.12 | 26.73 | 26.89 | 26.96 | 25.08 | 26.40 | 25.36 | 24.50 | 23.64 | — | — |
| 112 | 26.42 | 26.27 | 23.49 | 26.92 | 26.84 | 26.52 | 26.44 | 26.63 | 26.15 | 26.20 | 26.84 | — | — |
| 113 | 25.98 | 25.90 | 26.17 | 26.71 | 26.57 | 27.24 | 26.82 | 26.16 | 25.76 | 25.56 | 25.05 | — | — |
| 114 | 25.74 | 26.08 | 25.59 | 24.90 | 26.88 | 26.78 | 26.61 | 27.50 | 26.86 | 25.56 | 26.05 | — | — |
| 115 | 25.69 | 26.54 | 27.43 | 26.26 | 25.81 | 26.29 | 26.05 | 26.51 | 26.67 | — | — | — | — |
| N | 15 | 15 | 15 | 15 | 15 | 15 | 15 | 15 | 15 | 12 | 10 | 1 | 1 |
| Mean | 26.07 | 26.07 | 25.55 | 26.43 | 26.84 | 26.96 | 26.19 | 26.51 | 26.20 | 25.97 | 25.26 | 28.58 | 24.03 |
| SD | 1.21 | 1.09 | 1.70 | 1.01 | 0.97 | 0.88 | 1.01 | 0.94 | 1.01 | 1.30 | 1.40 | — | — |
| Min | 24.23 | 23.08 | 21.69 | 24.39 | 24.72 | 24.80 | 23.78 | 24.40 | 24.60 | 24.50 | 23.07 | 28.58 | 24.03 |
| Median | 25.74 | 26.27 | 25.65 | 26.38 | 26.88 | 26.78 | 26.05 | 26.40 | 26.15 | 25.56 | 25.47 | 28.58 | 24.03 |
| Max | 27.90 | 27.39 | 27.74 | 28.21 | 28.14 | 28.08 | 27.80 | 28.54 | 27.98 | 28.87 | 26.95 | 28.58 | 24.03 |
| CV % | 4.6 | 4.2 | 6.7 | 3.8 | 3.6 | 3.2 | 3.9 | 3.6 | 3.8 | 5.0 | 5.5 | — | — |

“—” = Not calculated; total ABT-874 serum concentration was below LOQ of 15 μg/mL.

TABLE 5
Individual and Summary % Glycoform Results for NGA2F-GlcNac

| | % NGA2F-GlcNac | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 3.01 | 3.24 | 2.47 | 2.92 | 3.08 | 2.99 | 3.21 | 2.93 | 3.13 | 3.22 | — | — | — |
| 102 | 3.07 | 2.99 | 3.09 | 3.10 | 3.02 | 2.96 | 2.86 | 2.79 | 3.00 | 2.84 | — | — | — |
| 103 | 3.27 | 3.08 | 3.21 | 3.31 | 3.08 | 3.16 | 3.02 | 2.88 | 2.72 | — | — | — | — |
| 104 | 3.13 | 2.83 | 3.02 | 2.91 | 2.97 | 2.67 | 2.85 | 3.29 | 3.12 | — | — | — | — |
| 105 | 3.14 | 3.07 | 3.04 | 3.05 | 3.12 | 3.09 | 3.15 | 3.14 | 3.08 | 2.97 | 3.37 | — | — |
| 106 | 3.22 | 3.18 | 3.42 | 3.48 | 3.05 | 3.36 | 3.07 | 3.05 | 3.15 | 2.88 | 2.89 | — | — |
| 107 | 2.95 | 3.46 | 3.37 | 3.24 | 3.08 | 3.06 | 2.94 | 2.62 | 2.82 | 2.90 | 2.67 | 2.38 | 2.90 |
| 108 | 4.34 | 3.36 | 3.60 | 3.33 | 3.20 | 3.47 | 3.42 | 3.34 | 3.60 | 3.27 | 3.24 | — | — |
| 109 | 4.21 | 3.53 | 4.82 | 3.57 | 3.72 | 3.32 | 3.68 | 3.35 | 3.88 | 3.69 | 3.58 | — | — |
| 110 | 3.79 | 3.49 | 3.92 | 3.23 | 3.61 | 3.34 | 3.41 | 3.47 | 3.15 | 3.39 | 3.84 | — | — |
| 111 | 4.27 | 3.66 | 3.84 | 3.49 | 3.81 | 3.44 | 3.59 | 3.20 | 3.60 | 3.59 | 3.86 | — | — |
| 112 | 3.78 | 3.62 | 3.06 | 3.43 | 3.62 | 3.61 | 3.67 | 3.52 | 3.79 | 3.38 | 3.40 | — | — |
| 113 | 4.62 | 4.23 | 4.28 | 4.72 | 4.13 | 3.94 | 3.83 | 3.59 | 4.22 | 4.10 | 4.92 | — | — |
| 114 | 4.41 | 4.27 | 4.53 | 3.68 | 3.83 | 4.18 | 3.84 | 3.86 | 4.11 | 4.21 | 3.58 | — | — |
| 115 | 4.07 | 3.85 | 3.30 | 3.66 | 3.52 | 3.66 | 3.35 | 3.35 | 3.19 | — | — | — | — |
| N | 15 | 15 | 15 | 15 | 15 | 15 | 15 | 15 | 15 | 12 | 10 | 1 | 1 |
| Mean | 3.69 | 3.46 | 3.53 | 3.41 | 3.39 | 3.35 | 3.33 | 3.23 | 3.37 | 3.37 | 3.54 | 2.38 | 2.90 |
| SD | 0.60 | 0.42 | 0.64 | 0.44 | 0.38 | 0.39 | 0.34 | 0.33 | 0.46 | 0.46 | 0.62 | — | — |
| Min | 2.95 | 2.83 | 2.47 | 2.91 | 2.97 | 2.67 | 2.85 | 2.62 | 2.72 | 2.84 | 2.67 | 2.38 | 2.90 |
| Median | 3.78 | 3.46 | 3.37 | 3.33 | 3.20 | 3.34 | 3.35 | 3.29 | 3.15 | 3.33 | 3.49 | 2.38 | 2.90 |
| Max | 4.62 | 4.27 | 4.82 | 4.72 | 4.13 | 4.18 | 3.84 | 3.86 | 4.22 | 4.21 | 4.92 | 2.38 | 2.90 |
| CV % | 16.2 | 12.3 | 18.1 | 12.8 | 11.1 | 11.7 | 10.3 | 10.3 | 13.8 | 13.6 | 17.4 | — | — |

“—” = Not calculated; total ABT-874 serum concentration was below LOQ of 15 μg/mL.

TABLE 6
Individual and Summary % Glycoform Results for NA1F-GlcNac

| | % NA1F-GlcNac | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 3.39 | 3.33 | 7.52 | 4.52 | 3.17 | 3.43 | 3.16 | 3.62 | 3.17 | 3.81 | — | — | — |
| 102 | 3.16 | 3.82 | 3.47 | 2.78 | 3.13 | 2.99 | 3.92 | 4.58 | 3.14 | 2.84 | — | — | — |
| 103 | 3.14 | 3.33 | 3.01 | 2.92 | 2.83 | 2.87 | 2.87 | 3.33 | 2.74 | — | — | — | — |
| 104 | 3.18 | 4.05 | 4.22 | 4.97 | 3.20 | 4.31 | 4.55 | 3.38 | 3.35 | — | — | — | — |
| 105 | 3.94 | 3.68 | 4.03 | 3.30 | 3.39 | 2.74 | 2.52 | 3.49 | 3.46 | 3.14 | 2.49 | — | — |
| 106 | 5.77 | 5.19 | 4.87 | 5.63 | 5.64 | 4.60 | 3.94 | 4.76 | 4.83 | 3.34 | 4.22 | — | — |
| 107 | 7.03 | 6.65 | 7.07 | 6.89 | 5.54 | 4.47 | 4.49 | 2.70 | 4.79 | 4.15 | 4.70 | 3.46 | 6.27 |
| 108 | 4.13 | 4.52 | 4.20 | 4.07 | 4.61 | 3.79 | 3.89 | 3.61 | 3.35 | 3.64 | 3.58 | — | — |
| 109 | 3.40 | 5.12 | 8.34 | 3.45 | 3.21 | 3.29 | 4.57 | 3.17 | 3.47 | 3.33 | 3.84 | — | — |
| 110 | 3.92 | 3.70 | 3.86 | 3.74 | 2.45 | 3.04 | 3.46 | 2.93 | 2.86 | 2.76 | 3.85 | — | — |
| 111 | 3.82 | 4.68 | 5.84 | 3.62 | 2.82 | 2.89 | 3.91 | 3.10 | 3.10 | 3.34 | 4.06 | — | — |
| 112 | 2.82 | 3.44 | 6.11 | 3.12 | 3.09 | 3.29 | 3.54 | 2.82 | 2.76 | 3.16 | 3.18 | — | — |
| 113 | 2.33 | 3.57 | 3.85 | 2.82 | 3.16 | 2.68 | 2.51 | 3.18 | 3.09 | 2.94 | 2.54 | — | — |
| 114 | 2.86 | 3.03 | 3.98 | 4.54 | 3.35 | 2.88 | 3.37 | 2.43 | 2.70 | 3.59 | 3.25 | — | — |
| 115 | 3.43 | 2.71 | 2.70 | 3.40 | 3.10 | 3.08 | 3.49 | 2.86 | 2.85 | — | — | — | — |
| N | 15 | 15 | 15 | 15 | 15 | 15 | 15 | 15 | 15 | 12 | 10 | 1 | 1 |
| Mean | 3.75 | 4.05 | 4.87 | 3.98 | 3.51 | 3.36 | 3.61 | 3.33 | 3.31 | 3.34 | 3.57 | 3.46 | 6.27 |
| SD | 1.20 | 1.02 | 1.71 | 1.16 | 0.96 | 0.64 | 0.67 | 0.64 | 0.66 | 0.41 | 0.71 | — | — |
| Min | 2.33 | 2.71 | 2.70 | 2.78 | 2.45 | 2.68 | 2.51 | 2.43 | 2.70 | 2.76 | 2.49 | 3.46 | 6.27 |
| Median | 3.40 | 3.70 | 4.20 | 3.62 | 3.17 | 3.08 | 3.54 | 3.18 | 3.14 | 3.34 | 3.71 | 3.46 | 6.27 |
| Max | 7.03 | 6.65 | 8.34 | 6.89 | 5.64 | 4.60 | 4.57 | 4.76 | 4.83 | 4.15 | 4.70 | 3.46 | 6.27 |
| CV % | 31.9 | 25.2 | 35.1 | 29.0 | 27.3 | 19.1 | 18.4 | 19.2 | 19.9 | 12.3 | 20.0 | — | — |

“—” = Not calculated; total ABT-874 serum concentration was below LOQ of 15 μg/mL.

TABLE 7
Individual and Summary % Glycoform Results for NA2F

| | % NA2F | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 4.89 | 5.19 | 4.81 | 5.05 | 5.36 | 4.82 | 3.84 | 4.18 | 4.26 | 3.92 | — | — | — |
| 102 | 3.96 | 3.88 | 3.83 | 4.20 | 4.20 | 4.31 | 4.04 | 3.77 | 4.57 | 5.25 | — | — | — |
| 103 | 3.84 | 3.81 | 3.95 | 4.08 | 4.09 | 4.01 | 3.64 | 3.69 | 3.86 | — | — | — | — |
| 104 | 4.36 | 4.11 | 4.29 | 4.25 | 4.61 | 4.28 | 3.59 | 4.14 | 3.79 | — | — | — | — |
| 105 | 4.25 | 4.31 | 4.38 | 4.17 | 4.15 | 4.37 | 4.21 | 4.42 | 4.34 | 4.48 | 4.80 | — | — |
| 106 | 3.90 | 3.82 | 4.31 | 4.03 | 3.98 | 4.44 | 4.70 | 5.16 | 5.73 | 7.39 | 7.74 | — | — |
| 107 | 4.27 | 4.17 | 3.73 | 3.49 | 4.63 | 3.60 | 3.44 | 4.56 | 3.86 | 4.66 | 4.01 | 3.92 | 10.81 |
| 108 | 6.54 | 5.00 | 5.42 | 4.90 | 4.62 | 5.05 | 4.79 | 4.74 | 5.07 | 4.47 | 4.48 | — | — |
| 109 | 8.60 | 7.42 | 9.58 | 6.45 | 7.08 | 5.94 | 8.55 | 7.28 | 8.53 | 7.22 | 8.32 | — | — |
| 110 | 5.15 | 5.18 | 5.14 | 4.73 | 4.78 | 5.17 | 5.00 | 4.87 | 4.75 | 6.81 | 6.31 | — | — |
| 111 | 6.12 | 5.58 | 6.51 | 5.05 | 5.75 | 5.37 | 5.79 | 5.68 | 5.74 | 6.11 | 6.89 | — | — |
| 112 | 5.08 | 5.02 | 4.92 | 5.15 | 5.34 | 5.21 | 5.10 | 5.31 | 5.40 | 6.13 | 6.81 | — | — |
| 113 | 5.31 | 5.20 | 5.35 | 5.02 | 5.18 | 4.75 | 5.23 | 4.33 | 4.51 | 4.91 | 6.13 | — | — |
| 114 | 5.78 | 5.62 | 5.92 | 5.16 | 5.04 | 5.79 | 5.02 | 5.04 | 5.10 | 4.85 | 4.75 | — | — |
| 115 | 4.72 | 4.54 | 4.02 | 4.62 | 6.12 | 4.64 | 4.00 | 4.20 | 5.33 | — | — | — | — |
| N | 15 | 15 | 15 | 15 | 15 | 15 | 15 | 15 | 15 | 12 | 10 | 1 | 1 |
| Mean | 5.12 | 4.86 | 5.08 | 4.69 | 5.00 | 4.78 | 4.73 | 4.76 | 4.99 | 5.52 | 6.02 | 3.92 | 10.81 |
| SD | 1.26 | 0.95 | 1.48 | 0.70 | 0.85 | 0.65 | 1.27 | 0.90 | 1.18 | 1.17 | 1.46 | — | — |
| Min | 3.84 | 3.81 | 3.73 | 3.49 | 3.98 | 3.60 | 3.44 | 3.69 | 3.79 | 3.92 | 4.01 | 3.92 | 10.81 |
| Median | 4.89 | 5.00 | 4.81 | 4.73 | 4.78 | 4.75 | 4.70 | 4.56 | 4.75 | 5.08 | 6.22 | 3.92 | 10.81 |
| Max | 8.60 | 7.42 | 9.58 | 6.45 | 7.08 | 5.94 | 8.55 | 7.28 | 8.53 | 7.39 | 8.32 | 3.92 | 10.81 |
| CV % | 24.7 | 19.5 | 29.2 | 15.0 | 17.0 | 13.6 | 26.8 | 18.8 | 23.6 | 21.3 | 24.3 | — | — |

“—” = Not calculated; total ABT-874 serum concentration was below LOQ of 15 μg/mL.

TABLE 8
Individual and Summary % Glycoform Results for M5

| | % M5 | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 5.53 | 5.95 | 5.04 | 5.15 | 4.80 | 4.16 | 3.76 | 3.55 | 3.39 | 3.15 | — | — | — |
| 102 | 4.94 | 4.87 | 4.92 | 4.49 | 4.10 | 3.90 | 3.28 | 3.03 | 3.13 | 2.71 | — | — | — |
| 103 | 4.77 | 4.91 | 4.52 | 4.29 | 3.65 | 3.61 | 3.08 | 3.00 | 2.52 | — | — | — | — |
| 104 | 4.98 | 4.81 | 4.93 | 4.45 | 4.19 | 3.75 | 3.02 | 3.49 | 2.99 | — | — | — | — |
| 105 | 5.10 | 5.15 | 5.03 | 4.77 | 4.18 | 4.14 | 3.75 | 3.76 | 3.66 | 3.07 | 3.25 | — | — |
| 106 | 4.74 | 4.35 | 4.77 | 3.88 | 3.14 | 3.58 | 3.41 | 3.38 | 3.39 | 2.99 | 3.28 | — | — |
| 107 | 4.65 | 5.13 | 5.37 | 4.87 | 4.48 | 3.26 | 2.97 | 3.88 | 2.79 | 2.69 | 2.32 | 1.40 | 4.02 |
| 108 | 5.01 | 5.79 | 5.86 | 4.91 | 3.91 | 4.22 | 3.98 | 3.78 | 4.01 | 3.32 | 3.09 | — | — |
| 109 | 4.97 | 6.15 | 4.81 | 4.41 | 4.30 | 3.42 | 3.91 | 3.75 | 3.80 | 3.17 | 2.89 | — | — |
| 110 | 5.70 | 5.51 | 4.91 | 3.80 | 4.06 | 3.72 | 3.17 | 3.02 | 2.51 | 2.85 | 3.08 | — | — |
| 111 | 5.46 | 5.71 | 4.62 | 4.14 | 4.11 | 3.47 | 3.29 | 3.31 | 3.04 | 2.79 | 3.21 | — | — |
| 112 | 5.84 | 5.89 | 5.28 | 4.55 | 4.85 | 4.27 | 3.80 | 3.67 | 3.63 | 2.91 | 3.04 | — | — |
| 113 | 5.54 | 5.65 | 4.94 | 4.87 | 4.55 | 4.28 | 4.01 | 3.46 | 3.62 | 3.32 | 3.74 | — | — |
| 114 | 4.95 | 5.33 | 5.40 | 4.40 | 4.12 | 4.22 | 3.80 | 3.75 | 3.72 | 3.37 | 2.71 | — | — |
| 115 | 5.03 | 5.33 | 4.48 | 4.69 | 3.84 | 3.77 | 3.20 | 2.95 | 2.59 | — | — | — | — |
| N | 15 | 15 | 15 | 15 | 15 | 15 | 15 | 15 | 15 | 12 | 10 | 1 | 1 |
| Mean | 5.15 | 5.37 | 4.99 | 4.51 | 4.15 | 3.85 | 3.50 | 3.45 | 3.25 | 3.03 | 3.06 | 1.40 | 4.02 |
| SD | 0.37 | 0.50 | 0.37 | 0.38 | 0.43 | 0.34 | 0.37 | 0.32 | 0.50 | 0.24 | 0.38 | — | — |
| Min | 4.65 | 4.35 | 4.48 | 3.80 | 3.14 | 3.26 | 2.97 | 2.95 | 2.51 | 2.69 | 2.32 | 1.40 | 4.02 |
| Median | 5.01 | 5.33 | 4.93 | 4.49 | 4.12 | 3.77 | 3.41 | 3.49 | 3.39 | 3.03 | 3.09 | 1.40 | 4.02 |
| Max | 5.84 | 6.15 | 5.86 | 5.15 | 4.85 | 4.28 | 4.01 | 3.88 | 4.01 | 3.37 | 3.74 | 1.40 | 4.02 |
| CV % | 7.2 | 9.4 | 7.3 | 8.5 | 10.5 | 8.9 | 10.7 | 9.4 | 15.2 | 8.0 | 12.3 | — | — |

“—” = Not calculated; total ABT-874 serum concentration was below LOQ of 15 μg/mL.

TABLE 9
Individual and Summary % Glycoform Results for M6

| | % M6 | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 2.75 | 2.09 | 2.84 | 2.12 | 1.90 | 1.67 | 1.11 | 1.30 | 1.07 | 1.26 | — | — | — |
| 102 | 2.08 | 1.90 | 1.30 | 0.93 | 1.26 | 1.10 | 1.76 | 1.10 | 1.61 | 0.77 | — | — | — |
| 103 | 2.42 | 1.72 | 1.24 | 1.36 | 1.05 | 1.18 | 1.06 | 1.69 | 0.85 | — | — | — | — |
| 104 | 2.30 | 1.67 | 1.73 | 1.77 | 1.88 | 1.76 | 2.67 | 2.36 | 1.93 | — | — | — | — |
| 105 | 2.69 | 1.86 | 1.91 | 1.51 | 1.22 | 1.24 | 1.15 | 1.97 | 1.77 | 1.47 | 1.83 | — | — |
| 106 | 4.01 | 2.54 | 2.66 | 3.10 | 2.42 | 2.07 | 1.97 | 3.07 | 3.07 | 1.91 | 2.27 | — | — |
| 107 | 3.18 | 2.78 | 2.41 | 2.74 | 2.28 | 1.35 | 1.14 | 1.56 | 1.98 | 2.06 | 2.01 | 0.98 | 5.72 |
| 108 | 3.83 | 2.95 | 2.84 | 2.42 | 2.62 | 2.00 | 2.41 | 2.22 | 1.96 | 1.78 | 2.11 | — | — |
| 109 | 3.83 | 4.53 | 6.61 | 2.18 | 2.12 | 1.69 | 4.03 | 2.46 | 3.69 | 2.96 | 3.62 | — | — |
| 110 | 3.81 | 2.64 | 2.37 | 1.53 | 1.38 | 1.95 | 1.85 | 1.63 | 1.47 | 1.71 | 2.93 | — | — |
| 111 | 4.10 | 3.46 | 3.89 | 1.84 | 2.57 | 2.00 | 3.02 | 2.28 | 2.35 | 2.88 | 3.86 | — | — |
| 112 | 2.41 | 2.29 | 4.85 | 1.77 | 2.01 | 2.08 | 1.90 | 1.88 | 2.00 | 2.14 | 1.80 | — | — |
| 113 | 2.31 | 2.52 | 1.99 | 1.55 | 1.62 | 1.67 | 1.68 | 1.35 | 1.68 | 1.14 | 1.64 | — | — |
| 114 | 3.08 | 2.43 | 2.86 | 2.70 | 1.99 | 2.14 | 1.68 | 1.37 | 1.97 | 2.02 | 1.74 | — | — |
| 115 | 2.76 | 1.79 | 0.90 | 2.23 | 2.16 | 1.35 | 1.27 | 1.15 | 1.08 | — | — | — | — |
| N | 15 | 15 | 15 | 15 | 15 | 15 | 15 | 15 | 15 | 12 | 10 | 1 | 1 |
| Mean | 3.04 | 2.48 | 2.69 | 1.98 | 1.90 | 1.68 | 1.91 | 1.83 | 1.90 | 1.84 | 2.38 | 0.98 | 5.72 |
| SD | 0.71 | 0.76 | 1.49 | 0.59 | 0.50 | 0.36 | 0.83 | 0.56 | 0.74 | 0.65 | 0.81 | — | — |
| Min | 2.08 | 1.67 | 0.90 | 0.93 | 1.05 | 1.10 | 1.06 | 1.10 | 0.85 | 0.77 | 1.64 | 0.98 | 5.72 |
| Median | 2.76 | 2.43 | 2.41 | 1.84 | 1.99 | 1.69 | 1.76 | 1.69 | 1.93 | 1.85 | 2.06 | 0.98 | 5.72 |
| Max | 4.10 | 4.53 | 6.61 | 3.10 | 2.62 | 2.14 | 4.03 | 3.07 | 3.69 | 2.96 | 3.86 | 0.98 | 5.72 |
| CV % | 23.3 | 30.7 | 55.2 | 29.7 | 26.2 | 21.3 | 43.4 | 30.8 | 38.8 | 35.3 | 33.9 | — | — |

“—” = Not calculated; total ABT-874 serum concentration was below LOQ of 15 μg/mL.

TABLE 10
Individual and Summary % Glycoform Results for M7

| | % M7 | | | | | | | | | | | | |
|---|---|---|---|---|---|---|---|---|---|---|---|---|---|
| | Planned Time (Days) | | | | | | | | | | | | |
| Subject | 0.02 | 0.25 | 0.5 | 1 | 1.5 | 2 | 3 | 5 | 7 | 10 | 14 | 21 | 28 |
| 101 | 0.66 | 0.57 | 0.44 | 0.29 | 0.21 | 0.20 | 0.12 | 0.11 | 0.08 | 0.05 | — | — | — |
| 102 | 0.51 | 0.47 | 0.32 | 0.09 | 0.69 | 0.10 | 0.07 | 0.07 | 0.16 | 0.27 | — | — | — |
| 103 | 0.42 | 0.38 | 0.24 | 0.27 | 0.18 | 0.23 | 0.06 | 0.17 | 0.05 | — | — | — | — |
| 104 | 0.45 | 0.20 | 0.23 | 0.23 | 0.13 | 0.05 | 0.67 | 0.63 | 0.27 | — | — | — | — |
| 105 | 0.52 | 0.32 | 0.21 | 0.08 | 0.09 | 0.09 | 0.09 | 0.16 | 0.14 | 0.12 | 0.17 | — | — |
| 106 | 1.02 | 0.87 | 0.94 | 1.09 | 0.75 | 0.79 | 0.56 | 1.10 | 1.01 | 1.23 | 1.04 | — | — |
| 107 | 0.71 | 0.34 | 0.60 | 0.23 | 0.28 | 0.14 | 0.76 | 0.15 | 0.68 | 0.86 | 0.89 | 0.77 | 1.99 |
| 108 | 1.79 | 1.22 | 1.00 | 0.77 | 1.13 | 0.52 | 0.73 | 0.61 | 0.38 | 0.66 | 0.59 | — | — |
| 109 | 0.96 | 2.08 | 2.61 | 0.25 | 0.29 | 0.24 | 1.35 | 0.46 | 0.66 | 0.28 | 0.55 | — | — |
| 110 | 1.87 | 1.08 | 0.98 | 0.34 | 0.39 | 0.71 | 0.79 | 0.69 | 0.69 | 1.03 | 1.58 | — | — |
| 111 | 1.34 | 1.10 | 0.77 | 0.25 | 0.24 | 0.33 | 1.17 | 0.09 | 0.37 | 0.64 | 1.09 | — | — |
| 112 | 1.11 | 0.96 | 2.36 | 0.48 | 0.49 | 0.81 | 0.56 | 0.43 | 0.44 | 0.50 | 0.43 | — | — |
| 113 | 1.21 | 1.19 | 0.80 | 0.53 | 0.75 | 0.64 | 0.64 | 0.25 | 0.16 | 0.38 | 0.39 | — | — |
| 114 | 1.37 | 1.13 | 0.91 | 1.56 | 0.49 | 0.77 | 0.46 | 0.31 | 0.56 | 0.57 | 0.45 | — | — |
| 115 | 1.21 | 0.83 | 0.44 | 0.44 | 0.69 | 0.21 | 0.25 | 0.08 | 0.17 | — | — | — | — |
| N | 15 | 15 | 15 | 15 | 15 | 15 | 15 | 15 | 15 | 12 | 10 | 1 | 1 |
| Mean | 1.01 | 0.85 | 0.86 | 0.46 | 0.45 | 0.39 | 0.55 | 0.35 | 0.39 | 0.55 | 0.72 | 0.77 | 1.99 |
| SD | 0.47 | 0.49 | 0.72 | 0.40 | 0.30 | 0.28 | 0.39 | 0.30 | 0.28 | 0.36 | 0.42 | — | — |
| Min | 0.42 | 0.20 | 0.21 | 0.08 | 0.09 | 0.05 | 0.06 | 0.07 | 0.05 | 0.05 | 0.17 | 0.77 | 1.99 |
| Median | 1.02 | 0.87 | 0.77 | 0.29 | 0.39 | 0.24 | 0.56 | 0.25 | 0.37 | 0.54 | 0.57 | 0.77 | 1.99 |
| Max | 1.87 | 2.08 | 2.61 | 1.56 | 1.13 | 0.81 | 1.35 | 1.10 | 1.01 | 1.23 | 1.58 | 0.77 | 1.99 |
| CV % | 46.2 | 57.8 | 84.2 | 87.4 | 65.3 | 73.3 | 70.9 | 84.0 | 72.4 | 65.6 | 59.1 | — | — |

“—” = Not calculated; total ABT-874 serum concentration was below LOQ of 15 μg/mL.

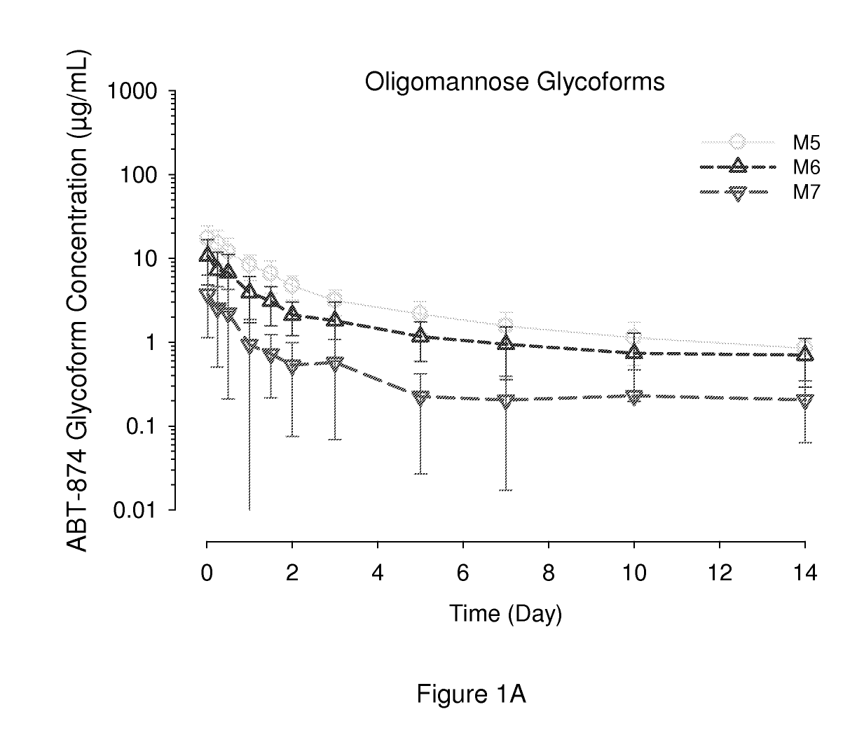

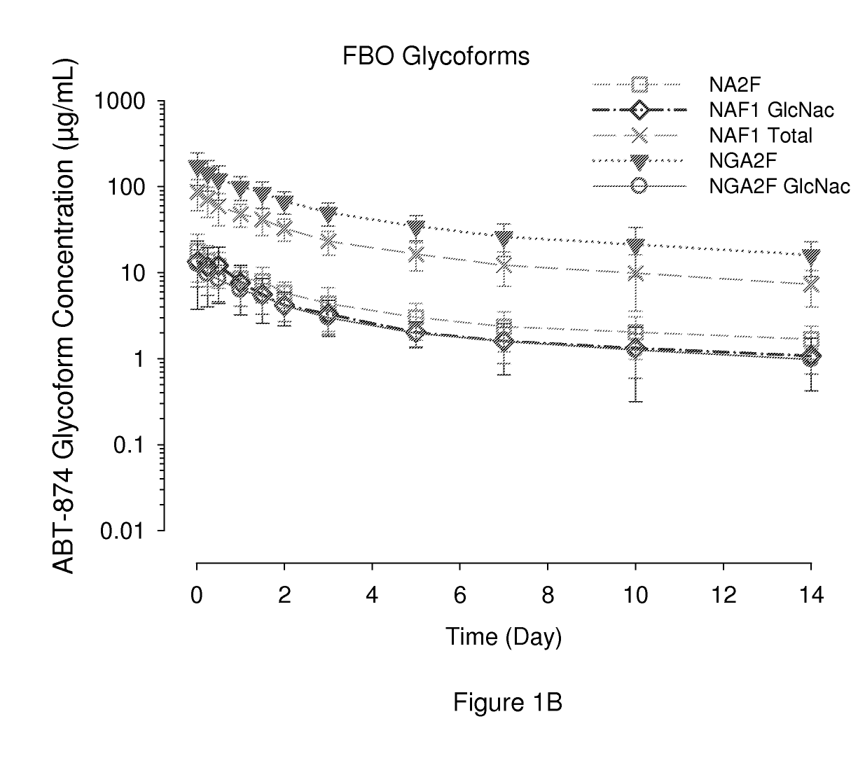

The mean±SD individual ABT-874 glycoforms serum concentrations over time following a single 700 mg IV infusion of ABT-874 are presented in FIG. 1. The mean serum concentrations of all of the FBO species appear to have a similar rate of decline over the 14 day period following dosing. Collectively, the mean concentrations of mannose species, and in particular M5, appear to decrease at a faster rate over the 14 day period following dose administration than the FBO species. The similarities in the pharmacokinetics of the FBO species and the mannose species support grouping the five FBO species and three mannose species into the two main groups for further analyses.

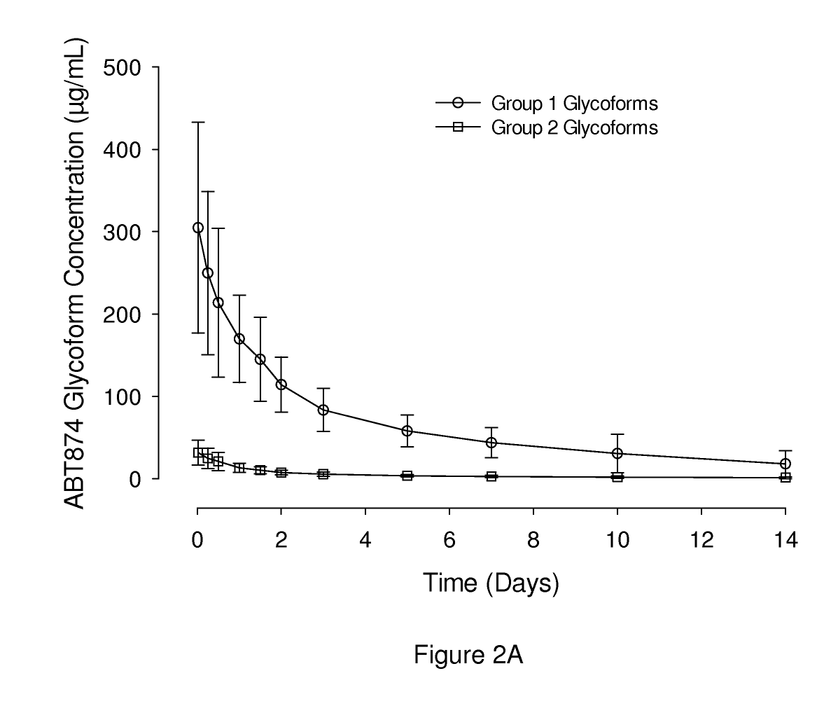

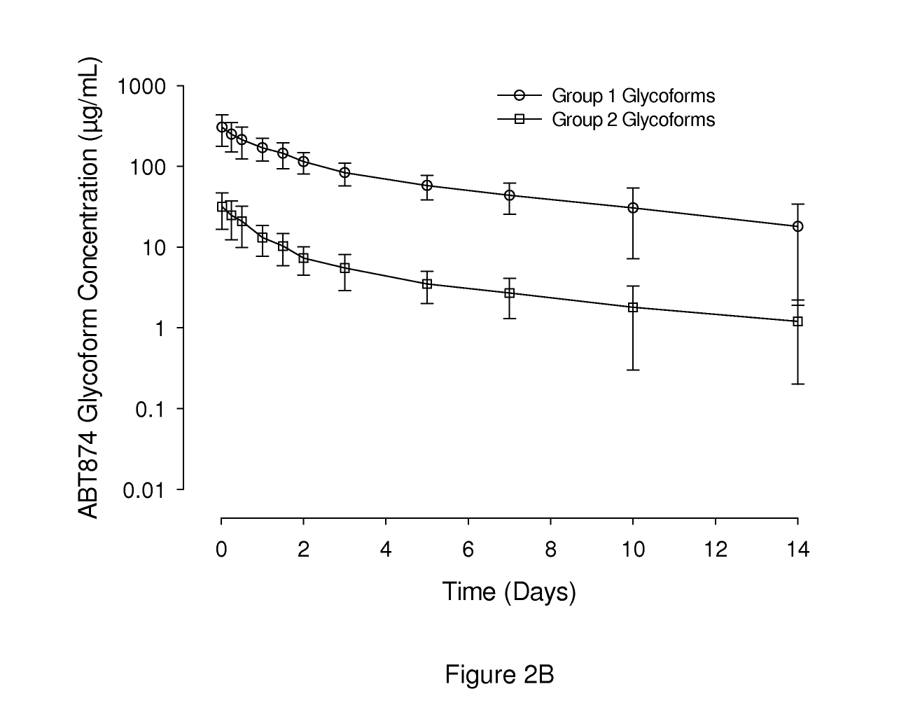

The mean±SD serum concentration-time profiles for glycoform Group 1 (FBO) and Group 2 (oligomannose) are presented on linear and log-linear scales following a single 700 mg IV infusion of ABT-874 in FIG. 2.

The median CL values for total ABT-874 (all glycoforms) and Group 1 were similar (<10% difference), while the median CL for Group 2 was ˜40% larger than both total ABT-874 and Group 1 median CL values. The median VI values were similar between Groups 1 and 2, and total ABT-874. This indicates that the elimination for Group 2 glycoforms is faster than Group 1 glycoforms and that the CL of total ABT-874 is driven primarily by Group 1.

### Population Pharmacokinetic Modeling

Based on earlier population pharmacokinetic modeling of ABT-874, the model building process started with a two compartment model, with linear elimination from a central compartment, and a peripheral compartment with one ETA for clearance (CL), and a proportional residual error model for both glycoform groups. The OFVs for the original model were 1265.497 for Group 1 (model run 100) and 525.374 for Group 2 (run101). Further pharmacokinetic parameters to be estimated were the volume of distribution of central compartment (V1), the inter compartmental clearance (Q), and the volume of distribution of the peripheral compartment (V2). The inclusion of a further exponential inter-individual term on V1 led to a drop of the OFV by 85.482 points for Group 1 (model run102), and by 31.523 points for the Group 2 (model run103), respectively. Because of the correlation between CL and V, the ‘BLOCK’ statement was used in the $OMEGA block of the models, which led to a further drop of OFVs by 10.858 (model run104) and 11.636 (model run 105) for Group 1 and Group 2, respectively. The extension of the residual error to a combined error model (proportional+additive) led to a further OFV improvement of 12.248 points (Group 1) and 36.584 points (Group 2). No further improvement of these models could be achieved, therefore models run 106 and run 107, were chosen as Final Models for Glycoform Group 1 and Group 2, respectively.

### Results

### Population Pharmacokinetic Model

In the population pharmacokinetic model, the ABT-874 serum concentrations were best described by a two-compartment model having linear elimination from a central compartment with a peripheral compartment.

The estimated pharmacokinetic parameter values and their associated variabilities from the ABT-874 models for both glycoform groups are listed in Table 11.

TABLE 11
Parameter Estimates and Variability for ABT-874 Glycoform
Group 1 and Group 2 Pharmacokinetics (Final Model)

| | | | | |
|---|---|---|---|---|
| | | | | 95% |
| | | Standard | | Confidence |
| Parameter | Estimate | Error (SE) | % RSE^{a} | Interval |

Group 1 (FBO)

| | | | | |
|---|---|---|---|---|
| CL (L/day) | 0.605 | 0.0461 | 7.62 | [0.515, 0.695] |
| CL (mL/h) | 25.2 | 1.92 | 7.62 | [21.4, 29.0] |
| Intersubject variance | 36.2 | NA | NA | NA |
| for CL^{b} | | | | |
| V1 (L) | 2.24 | 0.224 | 10.0 | [1.80, 2.68] |
| Intersubject variance | 41.8 | NA^{c} | NA | NA |
| for V^{b} | | | | |
| Q (L/day) | 0.631 | 0.054 | 8.56 | [0.525, 0.737] |
| V2 (L) | 1.82 | 0.0870 | 4.78 | [1.65, 1.99] |

Group 2 (Oligomannose)

| | | | | |
|---|---|---|---|---|
| CL (L/day) | 0.962 | 0.111 | 11.6 | [0.744, 1.18] |
| CL (mL/h) | 40.1 | 4.64 | 11.6 | [31.0, 49.2] |
| Intersubject variance | 47.3 | NA | NA | NA |
| for CL^{b} | | | | |
| V1 (L) | 2.45 | 0.365 | 14.9 | [1.74, 3.17] |
| Intersubject variance | 56.2 | NA | NA | NA |
| for V^{b} | | | | |
| Q (L/day) | 0.847 | 0.0749 | 8.84 | [0.700, 0.994] |
| V2 (L) | 2.95 | 0.308 | 10.4 | [2.35, 3.55] |

^{a}% RSE was estimated as the SE divided by the population estimate multiplied by 100.
^{b}Inter-subject Variance = SQRT(ETA) * 100.
^{c}NA = Not applicable.

Measures of variability were acceptable for all model parameters and the relative standard error (% RSE), was not larger than 15% for any model parameters in the final models.

Generally, the final pharmacokinetic model adequately described the observed serum concentrations in healthy subjects for both ABT-874 glycoform groups. The predicted vs. observed ABT-874 concentrations were scattered around the line of unity. The conditional weighted residuals did not show any major trend when plotted against predicted concentrations or sampling time indicating that the model was appropriately unbiased, and that the clearance of both ABT-874 glycoform groups was relatively time-independent.

Summary statistics for the pharmacokinetic model parameters are shown in Table 12.

TABLE 12
Summary Statistics for Model Parameters (Final Models)

| Parameter | Group | N | Mean | Std | % CV | Min | Median | Max |
|---|---|---|---|---|---|---|---|---|
| CL (mL/h) | 1 | 15 | 26.9 | 9.00 | 33.5 | 10.9 | 25.6 | 42.0 |
| CL (mL/h) | 2 | 15 | 42.8 | 20.1 | 47.1 | 17.7 | 35.8 | 80.7 |
| CL (mL/h) | all | 15 | 27.6 | 8.60 | 31.2 | 11.4 | 26.2 | 43.5 |
| V1 (L) | 1 | 15 | 2.51 | 1.07 | 42.7 | 1.11 | 2.23 | 4.38 |
| V1 (L) | 2 | 15 | 2.73 | 1.61 | 58.8 | 1.15 | 2.06 | 5.58 |
| V1 (L) | all | 15 | 2.56 | 1.21 | 47.1 | 1.11 | 2.20 | 4.74 |
| Q (mL/h) | 1 | 15 | 26.3 | NA | NA | NA | NA | NA |
| Q (mL/h) | 2 | 15 | 35.3 | NA | NA | NA | NA | NA |
| Q (mL/h) | all | 15 | 28.0 | NA | NA | NA | NA | NA |
| V2 (L) | 1 | 15 | 1.82 | NA | NA | NA | NA | NA |
| V2 (L) | 2 | 15 | 2.95 | NA | NA | NA | NA | NA |
| V2 (L) | all | 15 | 1.83 | NA | NA | NA | NA | NA |

Std = standard deviation.
Min = minimum;
max = maximum.
% CV = percent coefficient of variation.
NA = Not applicable.
Group 1 = FBO;
Group 2 = Oligomannose

The median CL values for total ABT-874 (all glycoforms) and Group 1 were similar (<10% difference), while the median CL for Group 2 was ˜40% larger than both total ABT-874 and Group 1 median CL values. The median V1 values were similar between Groups 1 and 2, and total ABT-874. This suggests that the elimination for Group 2 glycoforms is faster than Group 1 glycoforms and that the CL of total ABT-874 is driven primarily by Group 1.

### Model Evaluation

ABT-874 Pharmacokinetic Model

Goodness-of-Fit Plots

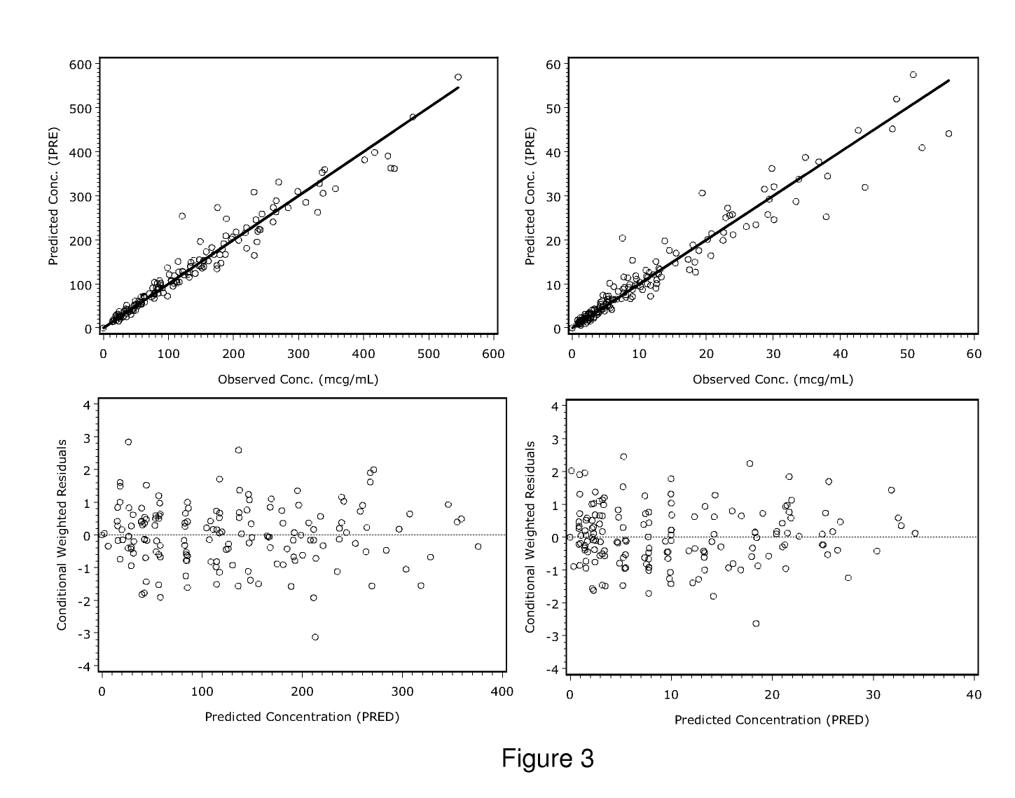

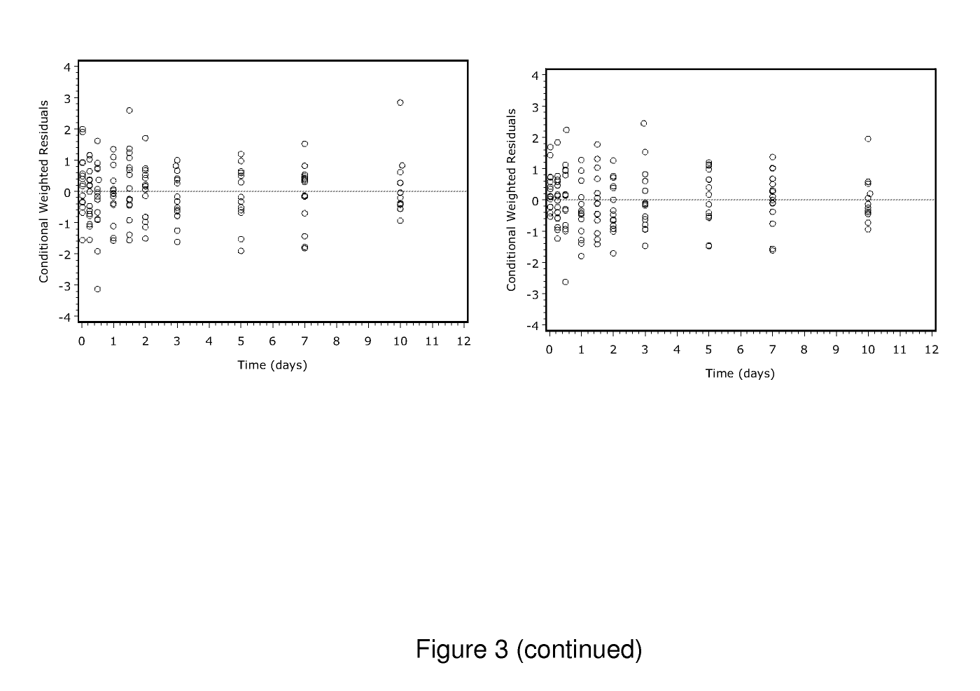

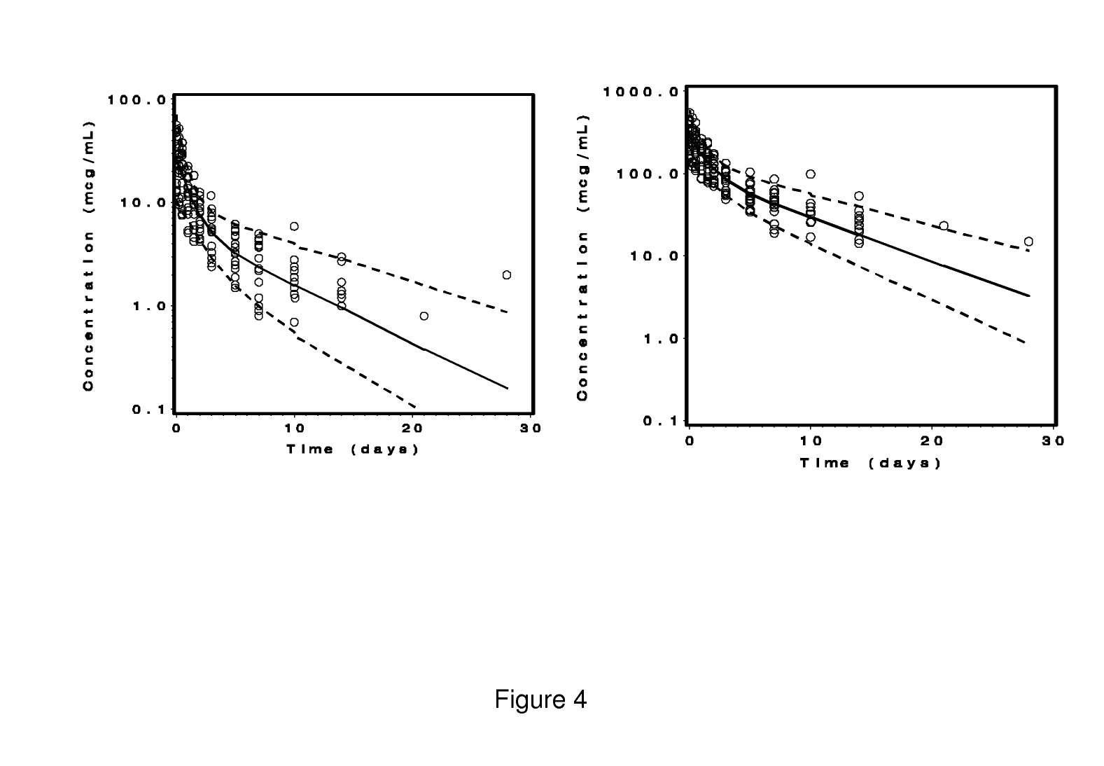

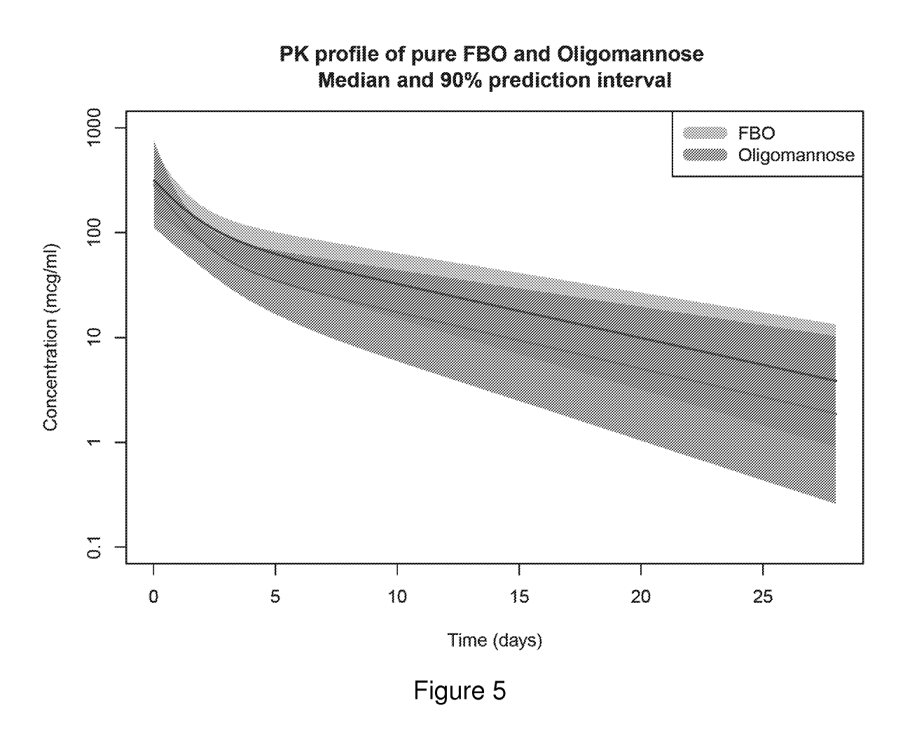

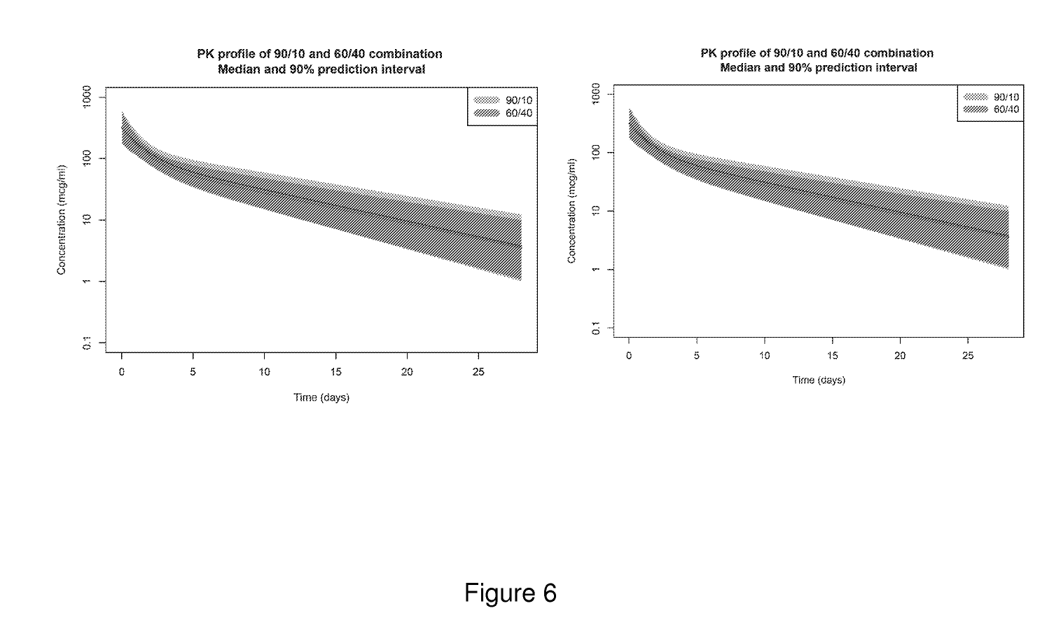

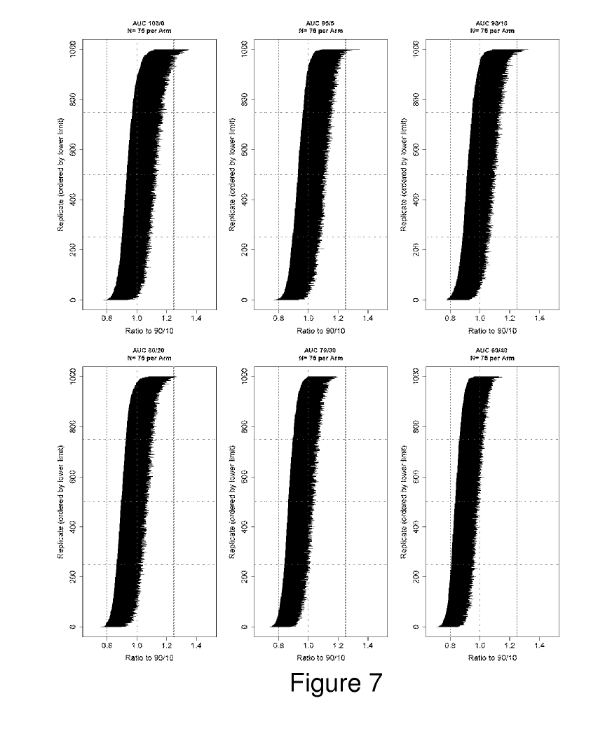

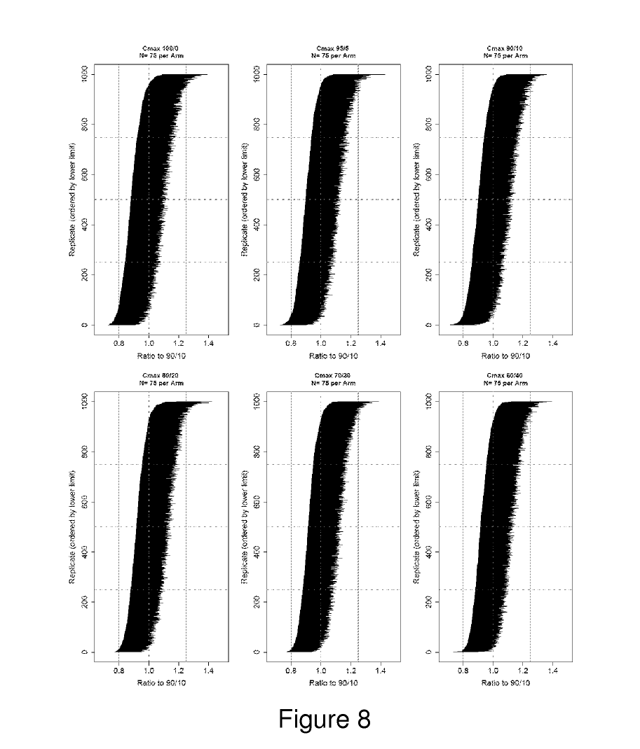

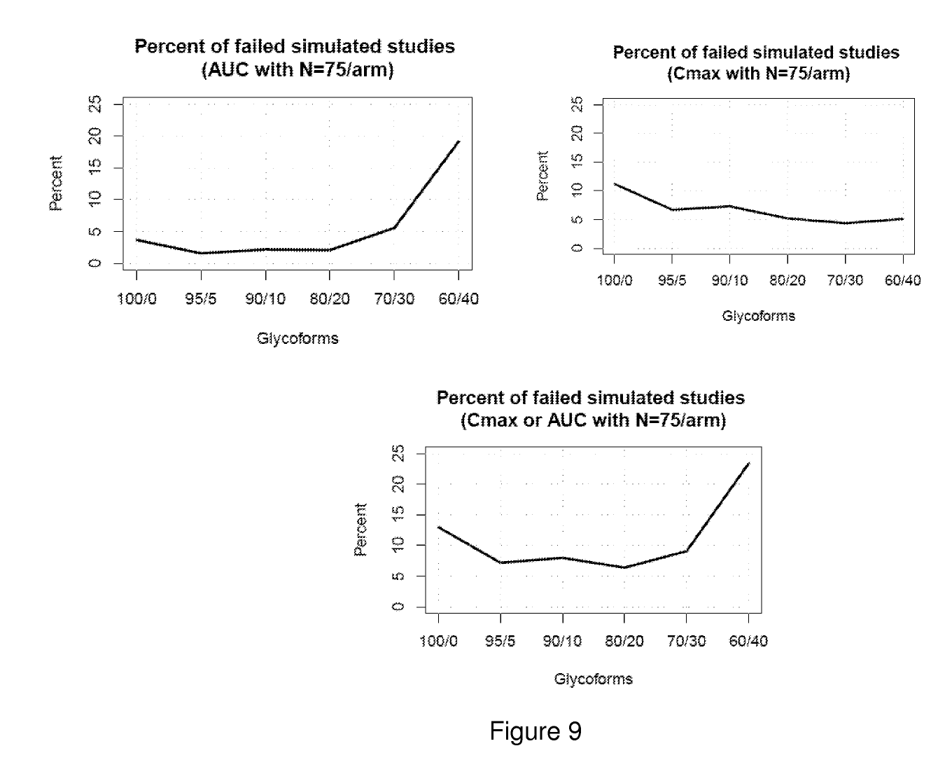

Inter-individual variabilities for ABT-874 CL and V1 were 36.2% and 41.8% for Group 1, and 47.3% and 56.2% for Group 2, respectively. The goodness-of-fit for the final model was evaluated graphically. The goodness-of-fit plots of the individual predicted ABT-874 concentrations versus the observed concentrations and the weight residuals versus time are presented in FIG. 3. The plots indicated that the model adequately described the observations over the entire ABT-874 serum concentration range since observed and predicted ABT-874 concentrations were randomly distributed across the line of unity (a straight line with zero intercept and a slope of one) and the plot of weighted residuals revealed no systemic trends versus population predicted concentrations or over time.

Visual Predictive Checks

The results of visual predictive checks with 1000 simulations stratified by glycoform groups are shown in FIG. 4. Overall, the variability in the observed data was described with good accuracy for both groups.

Bootstrap Evaluation

A total of 987 out of 1000 bootstrap replicates ran successfully for the Final Model of ABT-874 Group 1 and Group 2.

The estimated pharmacokinetic parameter values based on the original dataset were in good agreement with the medians of the parameter values estimated from the bootstrap replicates for both groups of glycoforms (Table 13). This agreement demonstrated that estimation of parameter values by the ABT-874 pharmacokinetic model for both glycoform groups was robust and based on the global minimum of the likelihood profile.

In accordance with the estimated standard errors of the estimate (SE) for pharmacokinetic parameters in the ABT-874 pharmacokinetic model, none of the 95% confidence intervals from the bootstrap validation for the four pharmacokinetic parameters included zero.

TABLE 13
Medians and 95% Confidence Intervals for ABT-874 Pharmacokinetic
Parameters Estimated From Bootstrap Evaluation

| | | Bootstrap Evaluation Results | |
|---|---|---|---|
| Pharmacokinetic | | Median | 95% Confidence |
| Parameter | Model Result | (N = 987) | Interval (N = 987) |

Group 1 (FBO)

| | | | |
|---|---|---|---|
| CL (L/day) | 0.605 | 0.600 | [0.480, 0.706] |
| CL (mL/h) | 25.2 | 25.0 | [20.0, 29.4] |
| V1 (L) | 2.24 | 2.23 | [1.75, 2.91] |
| Q (L/day) | 0.631 | 0.617 | [0.499, 0.725] |
| V2 (L) | 1.82 | 1.86 | [1.67, 2.56] |

Group 2 (Oligomannose)

| | | | |
|---|---|---|---|
| CL (L/day) | 0.962 | 0.931 | [0.466, 1.21] |
| CL (mL/h) | 40.1 | 38.8 | [19.4, 50.4] |
| V1 (L) | 2.45 | 2.41 | [1.64, 3.41] |
| Q (L/day) | 0.847 | 0.862 | [0.713, 1.07] |
| V2 (L) | 2.95 | 3.05 | [2.39, 9.95] |

### Simulations of ABT-874 Glycoform Pharmacokinetics: Bioequivalence Analyses

To understand the impact of varying percentages of glycoform groups on the pharmacokinetics of total ABT-874, simulations of bioequivalence studies using test products with different glycoform compositions were conducted including the 90/10 composition as reference. For illustrative purposes, ABT-874 pharmacokinetic profiles of pure 100% FBO and 100% oligomannose were simulated and plotted in FIG. 5. Pharmacokinetic profiles of the test products with 70/30 FBO/Oligomannose and 60/40 versus the reference product with 90/10 composition are shown in FIG. 6.

For the estimation of the effect of different compositions versus reference, the percentages of replicates with 90% confidence intervals outside of the 80% to 125% range were calculated and represented graphically (AUC_{0-28d}: FIG. 7, Cₘₐₓ: FIG. 7). The percentages of studies not meeting the bioequivalence criteria versus glycoform ratio are shown in FIG. 9.

Simulation results indicate that varying the total oligomannose percentage from 5% up to 30% would have minor impact on the pharmacokinetics of total ABT-874, as the 90% confidence interval for the AUC_{0-28d} and Cₘₐₓ ratios fit within the bioequivalence range for over 90% of the studies with sample sizes of 150 subjects (n=75 per arm). With 75 subjects per arm, increasing the percentages of oligomannoses beyond 40% would have a likelihood of not meeting bioequivalence criteria of more than 20%. The probability of meeting the bioequivalence criterion would increase with increased sample size.

In the present analyses of ABT-874 glycoform pharmacokinetics, two population PK models were constructed to describe the pharmacokinetics of fucosylated biantennary oligosaccharides (FBO) and oligomannose glycoforms. Similarities of both the biochemical properties (presence or absence of fucose) and preliminary pharmacokinetic analyses of the individual glycoforms support the grouping of the eight glycoforms into two major species. The two population PK models adequately described the pharmacokinetics of these two glycoforms groups and demonstrated that ABT-874 oligomannose glycoforms (Group 2) have an approximately 40% greater clearance than FBO glycoforms (Group 1).

In the clinical lots of ABT-874 used in human studies to date, the percentages of oligomannose species have been approximately 10% or less. In the current study, the composition of ABT-874 was approximately 90% FBO and 10% oligomannose. At this composition, the clearance estimates of the FBO group (Group 1), oligomannose group (Group 2), and total ABT-874 (all) demonstrated that the FBO group has similar clearance (26.9 mL/hr) to the total ABT-874 estimate (27.6 mL/hr), while the oligomannose group estimate was approximately 40% higher (42.8 mL/hr). This demonstrates that even with the increased clearance of the oligomannose group, total ABT-874 clearance is controlled primarily by the FBO group. Therefore, while the clearance of the oligomannose species is higher than the FBO glycoforms, there is minimal impact on the overall pharmacokinetics of total ABT-874, because they represent a smaller percentage of the ABT-874 glycoforms.

Simulations of bioequivalence studies were conducted to investigate the magnitude of change that would be necessary to influence the pharmacokinetics of total ABT-874. Results indicate that increasing the oligomannose species to approximately 30%, minimally increases the risk of bioequivalence study failure, as the percentages of studies with 90% confidence intervals for the ratios of AUC_{0-28d} and Cₘₐₓ falling outside of the 80% to 125% range are similar to those of ABT-874 product with 10% oligomannose species. When the percentages of oligomannose species increase above 30%, the risk of failing bioequivalence would increase. Therefore, an increase of oligomannose species two-fold (˜20%) over what has been used clinically would provide similar exposures to those of the clinical supply used in the current study (oligomannose ˜10%). These simulations support that changes in the composition of ABT-874 glycoforms of up to approximately 30% oligomannose would have minimal impact of the pharmacokinetics of total ABT-874.

### SUMMARY

A population pharmacokinetic analysis for the glycoforms of ABT-874 has been performed using serum concentration data from 15 subjects who received a single 700 mg ABT-874 IV infusion. Eight different glycoforms of ABT-874 were grouped based on their similar pharmacokinetics and biochemical properties, either as FBO oligosaccharides or oligomannoses, and were analyzed. The final population pharmacokinetic models for both glycoform groups are two-compartment models having linear elimination from a central compartment with a peripheral compartment and an inter-compartmental clearance, with two exponential inter-individual variability terms on the CL and V1 of the central compartment, a combined residual error model (with a proportional and an additive term). The reliability of the final models as well as the variability of pharmacokinetic parameters were confirmed by Goodness-Of-Fit Plots, by inspection of individual data plots, by bootstrap evaluation and visual predictive checks.

The final population pharmacokinetic models were used to simulate ABT-874 serum concentrations following administration of a drug product with composition similar to the one administered in this study (90% fucosylated biantennary, 10% oligomannose), and of hypothetical study drug products, consisting of varying compositions of glycoforms with oligomannose percentage ranging from 0% to 40%. Using the simulated subjects, replicates of parallel group bioequivalence studies were simulated. For each subject, AUC_{0-28d} and Cₘₐₓ were calculated. For each composition, the ratio relative to the reference composition (90/10) and its 90% confidence interval were calculated in each replicated study. The percentages of replicates with a 90% confidence interval of the ratio of AUC_{0-28d} and Cₘₐₓ between test and reference composition outside the 80% to 125% range were calculated. The simulation results demonstrate that varying the total oligomannose percentage from 0% up to 30% would have minor impact on the pharmacokinetics of total ABT-874.

### EQUIVALENTS

Those skilled in the art will recognize, or be able to ascertain using no more that routine experimentation, many equivalents to the specific embodiments of the invention described herein. Such equivalents are intended to be encompassed by the following claims.

1. A composition comprising a human antibody, or antigen binding portion thereof, the composition comprising
(a) a first level of the antibody, or antigen binding portion thereof, which is glycosylated at an N-linked glycosylation site on the Fc region with an oligomannose-type structure; and
(b) a second level of the antibody, or antigen binding portion thereof, which is glycosylated at the N-linked glycosylation site on the Fc region with a fucosylated biantennary oligosaccharide-type structure;
wherein the composition exhibits a desired rate of serum clearance.

2. The composition of claim 1, wherein the N-linked glycosylation site is an asparagine residue on the Fc region of the antibody.

3. The composition of claim 2, wherein the asparagine residue is Asn 297.

4. The composition of claim 1, wherein the oligomannose-type structure is independently selected from the group consisting of M5, M6, M7, M8, and M9.

5. The composition of claim 1, wherein the fucosylated biantennary oligosaccharide-type structure is independently selected from the group consisting of NGA2F, NA1F, NA2F, NGA2F-GlcNAc, and NA1F-GlcNAc.

6. The composition of claim 1, wherein the first level is about 0-100%.

7. The composition of claim 1, wherein the first level is about 10-30%.

8. (canceled)

9. The composition of claim 1, wherein the second level is about 0-100%.

10. The composition of claim 1, wherein the second level is about 70-90%.

11. (canceled)

12. The composition of claim 1, wherein the desired rate of serum clearance is a rapid rate of serum clearance.

13. (canceled)

14. (canceled)

15. (canceled)

16. (canceled)

17. The composition of claim 1, wherein the desired rate of serum clearance is a slow rate of serum clearance.

18. (canceled)

19. (canceled)

20. The composition of claim 1, wherein the antibody, or antigen binding portion thereof, comprises a λ light chain.

21. (canceled)

22. (canceled)

23. (canceled)

24. (canceled)

25. (canceled)

26. (canceled)

27. The composition of claim 1, wherein the antibody, or antigen binding portion thereof, is an anti-IL-12 antibody.

28. The composition of claim 1, wherein the antibody, or antigen binding portion thereof, is an anti-IL-23 antibody.

29. The composition of claim 1, wherein the antibody, or antigen binding portion thereof, is ABT-874 or a fragment thereof.

30. (canceled)

31. (canceled)

32. (canceled)

33. (canceled)

34. The composition of claim 1, wherein the antibody, or antigen binding portion thereof, is an antibody, or fragment thereof, selected from the group consisting of CNT01275, tositumomab, WRI-170, WO1, TNF-H9G1, THY-32, THY-29, TEL16, TEL14, Tel13, SM1, S1-1, RSP4, RH-14, RF-TS7, RF-SJ2, RF-SJ1, RF-AN, PR-TS2, PR-TS1, PR-SJ2, PR-SJ1, PHOX15, PAG-1, OG-31, NO. 13, NM3E2 SCFV, MUC1-1, MN215, MC116, MAD-2, MAB67, MAB63, MAB60, MAB59, MAB57, MAB56, MAB111, MAB107, L3055-BL, K6H6, K6F5, K5G5, K5C7, K5B8, K4B8, JAC-10, HUC, HMST-1, HIH2, HIH10, HBW4-1, HBP2, HA1, H6-3C4, H210, GP44, GG48, GG3, GAD-2, FOM-A, FOM-1, FOG1-A3, FOG-B, DPC, DPA, DOB1, DO1, CLL001, CLL-249, CD4-74, CB-201, C304 RF, BSA3, BO3, BO1, BEN-27, B-33, B-24, ANTI-TEST, ANTI-EST, ANTI-DIGB, ANTI-DIGA, AIG, 9604, 448.9G.F1, 33.H11, 32.B9, 24A5, 1B9/F2, 13E10, 123AV16-1, 11-50, and 1.32.

35. The composition of claim 1, wherein the composition further comprises an additional agent selected from the group consisting of a buffer, a polyol and a surfactant.

36. (canceled)

37. (canceled)

38. (canceled)

39. (canceled)

40. The composition of claim 1, wherein the concentration of the antibody, or antigen binding portion thereof, is about 0.1-250 mg/ml.

41. (canceled)

42. (canceled)

43. (canceled)

44. (canceled)

45. (canceled)

46. (canceled)

47. (canceled)

48. A composition comprising a human antibody, or antigen binding portion thereof, wherein the composition comprises
(a) about 0-100% of the antibody, or antigen binding portion thereof, which is glycosylated at an N-linked glycosylation site on the Fc region with an oligomannose-type structure; and
(b) about 0-100% of the antibody, or antigen binding portion thereof, which is glycosylated at the N-linked glycosylation site on the Fc region with a fucosylated biantennary oligosaccharide-type structure, wherein the composition exhibits a desired rate of serum clearance.

49. A composition comprising a human antibody, or antigen binding portion thereof, wherein the composition comprises
(a) about 10-30% of the antibody, or antigen binding portion thereof, which is glycosylated at an N-linked glycosylation site on the Fc region with an oligomannose-type structure; and
(b) about 70-90% of the antibody, or antigen binding portion thereof, which is glycosylated at the N-linked glycosylation site on the Fc region with a fucosylated biantennary oligosaccharide-type structure, wherein the composition exhibits a desired rate of serum clearance.

50. A composition comprising ABT-874, or antigen binding portion thereof, wherein
(a) about 0-100% of the ABT-874 is glycosylated at Asn 297 with an oligomannose structure that is independently selected from the group consisting of M5, M6, M7, M8 and M9; and
(b) about 0-100% of the ABT-874 is glycosylated at Asn 297 with a fucosylated biantennary oligosaccharide structure that is independently selected from the group consisting of NGA2F, NA1F, NA2F, NGA2F-GlcNAc, and NA1F-GlcNAc.

51. A composition comprising ABT-874, or antigen binding portion thereof, wherein
(a) about 10-30% of the ABT-874 is glycosylated at Asn 297 with an oligomannose structure that is independently selected from the group consisting of M5, M6, M7, M8 and M9; and
(b) about 70-90% of the ABT-874 is glycosylated at Asn 297 with a fucosylated biantennary oligosaccharide structure that is independently selected from the group consisting of NGA2F, NA1F, NA2F, NGA2F-GlcNAc, and NA1F-GlcNAc.

52. A method for modulating the pharmacokinetics of a composition comprising a human antibody, or antigen binding portion thereof, the method comprising
(a) modulating a first level of the antibody that is glycosylated at an N-linked glycosylation site on the Fc region with an oligomannose-type structure; and
(b) modulating a second level of the antibody that is glycosylated at the N-linked glycosylation site on the Fc region with a fucosylated biantennary oligosaccharide-type structure;
wherein the modulation of the first and second levels results in a desired rate of serum clearance, thereby modulating the pharmacokinetics of a composition comprising a human antibody, or antigen binding portion thereof.

53. The method of claim 52, wherein the N-linked glycosylation site is an asparagine residue on the Fc region of the antibody.

54. The method of claim 53, wherein the asparagine residue is Asn 297.

55. The method of claim 52, wherein the oligomannose-type structure is independently selected from the group consisting of M5, M6, M7, M8, and M9.

56. The method of claim 52, wherein the fucosylated biantennary oligosaccharide-type structure is independently selected from the group consisting of NGA2F, NA1F, NA2F, NGA2F-GlcNAc, and NA1F-GlcNAc.

57. The method of claim 52, wherein the first level is about 0-100%.

58. The method of claim 52, wherein the first level is about 10-30%.

59. (canceled)

60. The method of claim 52, wherein the second level is about 0-100%.

61. The method of claim 52, wherein the first level is about 10-30%.

62. (canceled)

63. The method of claim 52, wherein the desired rate of serum clearance is a rapid rate of serum clearance.

64. (canceled)

65. (canceled)

66. (canceled)

67. (canceled)

68. The method of claim 52, wherein the desired rate of serum clearance is a slow rate of serum clearance.

69. (canceled)

70. (canceled)

71. The method of claim 52, wherein the antibody, or antigen binding portion thereof, comprises a λ light chain.

72. The method of claim 52, wherein the antibody, or antigen binding portion thereof, comprises a heavy chain constant region selected from the group consisting of IgG1, IgG2, IgG3, and IgG4 constant regions.

73. (canceled)

74. The method of claim 52, wherein the antibody, or antigen binding portion thereof, comprises an IgG1 heavy chain constant region and a λ light chain.

75. (canceled)

76. (canceled)

77. (canceled)

78. The method of claim 52, wherein the antibody, or antigen binding portion thereof, is an anti-IL-12 antibody.

79. The method of claim 52, wherein the antibody, or antigen binding portion thereof, is an anti-IL-23 antibody.

80. The method of claim 52, wherein the antibody, or antigen binding portion thereof, is ABT-874 or a fragment thereof.

81. (canceled)

82. (canceled)

83. (canceled)

84. (canceled)

85. The composition of claim 52, wherein the antibody, or antigen binding portion thereof, is an antibody, or fragment thereof, selected from the group consisting of CNT01275, tositumomab, WRI-170, WO1, TNF-H9G1, THY-32, THY-29, TEL16, TEL14, Tel13, SM1, S1-1, RSP4, RH-14, RF-TS7, RF-SJ2, RF-SJ1, RF-AN, PR-TS2, PR-TS1, PR-SJ2, PR-SJ1, PHOX15, PAG-1, OG-31, NO. 13, NM3E2 SCFV, MUC1-1, MN215, MC116, MAD-2, MAB67, MAB63, MAB60, MAB59, MAB57, MAB56, MAB111, MAB107, L3055-BL, K6H6, K6F5, K5G5, K5C7, K5B8, K4B8, JAC-10, HUC, HMST-1, HIH2, HIH10, HBW4-1, HBP2, HA1, H6-3C4, H210, GP44, GG48, GG3, GAD-2, FOM-A, FOM-1, FOG1-A3, FOG-B, DPC, DPA, DOB1, DO1, CLL001, CLL-249, CD4-74, CB-201, C304 RF, BSA3, BO3, BO1, BEN-27, B-33, B-24, ANTI-TEST, ANTI-EST, ANTI-DIGB, ANTI-DIGA, AIG, 9604, 448.9G.F1, 33.H11, 32.B9, 24A5, 1B9/F2, 13E10, 123AV16-1, 11-50, and 1.32.

86. A method for modulating the pharmacokinetics of a composition comprising ABT-874, or an antigen-binding portion thereof, the method comprising
(a) modulating a first level of ABT-874, or an antigen-binding fragment thereof, that is glycosylated at an N-linked glycosylation site on the Fc region with an oligomannose-type structure that is independently selected from the group consisting of M5, M6, M7, M8 and M9; and
(b) modulating a second level ABT-874, or an antigen-binding fragment thereof, that is glycosylated at the N-linked glycosylation site on the Fc region with a fucosylated biantennary oligosaccharide-type structure that is independently selected from the group consisting of NGA2F, NA1F, NA2F, NGA2F-GlcNAc, and NA1F-GlcNAc;
wherein the modulation of the first and second levels results in a desired rate of serum clearance, thereby modulating the pharmacokinetics of a composition comprising ABT-874, or an antigen binding portion thereof.


SEQUENCE LISTING (675 sequences)

SEQ ID NO: 1 - protein, 6 aa, Homo sapiens
  MOD_RES (1)..(1): His or Ser
XGSXDX

SEQ ID NO: 2 - protein, 12 aa, Homo sapiens
  MOD_RES (2)..(2): Ser or Thr
QXYXXXXXXXXX

SEQ ID NO: 3 - protein, 17 aa, Homo sapiens
FIRYDGSNKYYADSVKG

SEQ ID NO: 4 - protein, 7 aa, Homo sapiens
  MOD_RES (1)..(1): Gly or Tyr
XNXXRPS

SEQ ID NO: 5 - protein, 9 aa, Homo sapiens
  MOD_RES (5)..(5): Ser or Glu
FTFSXYGMH

SEQ ID NO: 6 - protein, 13 aa, Homo sapiens
  MOD_RES (1)..(1): Ser or Thr
XGXXSNIXXXXVX

SEQ ID NO: 7 - protein, 115 aa, Homo sapiens
  MOD_RES (6)..(6): Gln or Glu
QVQLVXSGGGVVQPGXSLRLSCAASGFTFSXYGMHWVRQAPGKGLEWVAFIRYDGSNKYYADSBKGRFTISRDNSKNTLYLQMXSLRAEDTAVYYCXXXGSXDXWGQGTMVTVSS

SEQ ID NO: 8 - protein, 112 aa, Homo sapiens
  MOD_RES (1)..(1): Ser or Gln
XXVLTQPPSVSGXPGQRVTISCXGXXSNIXXXXVXWYQQLPGTAPKLLIYXNXXRPSGVPDRFSGSKSGTSASLAITGXQAEDEADYYCQXYXXXXXXXXXFGTGTKVTVLG

SEQ ID NO: 9 - protein, 6 aa, Homo sapiens
  MOD_RES (2)..(2): Gly, Val, Cys or His
HXXXXX

SEQ ID NO: 10 - protein, 12 aa, Homo sapiens
  MOD_RES (4)..(4): Asp or Ser
QSYXXXTHPALL

SEQ ID NO: 11 - protein, 17 aa, Homo sapiens
  MOD_RES (1)..(1): Phe, Thr or Tyr
XIXYXXSXKXYADSVKG

SEQ ID NO: 12 - protein, 7 aa, Homo sapiens
  MOD_RES (1)..(1): Gly, Tyr, Ser, Thr, Asn or Gln
XNDQRPS

SEQ ID NO: 13 - protein, 9 aa, Homo sapiens
  MOD_RES (4)..(5): Any amino acid
FTFXXXXMH

SEQ ID NO: 14 - protein, 13 aa, Homo sapiens
  MOD_RES (9)..(9): Ser, Cys, Arg, Asn, Asp or Thr
SGGRSNIGXXXVK

SEQ ID NO: 15 - protein, 114 aa, Homo sapiens
  MOD_RES (5)..(5): Gln or Glu
QVQVXSGGGVVQPGRSLRLSCAASGFTFSXYGMHWVRQAPGKGLEWVAFIRYDGSNKYYADSVKGRFTISRDNSKNTLYLQMXSLRAEDTAVYYCKTHGSHDNWGQGTMVTVSS

SEQ ID NO: 16 - protein, 112 aa, Homo sapiens
  MOD_RES (1)..(1): Ser or Gln
XXVLTQPPSVSGXPGQRVTISCSGXRSNIGSNTVKWYQQLPGTAPKLLIYXNDQRPSGVPDRFSGSKSGTSASLAITGXQAEDEADYYCQSYDRXTHPALLFGTGTKVTVLG

SEQ ID NO: 17 - protein, 6 aa, Homo sapiens
HGSHDN

SEQ ID NO: 18 - protein, 12 aa, Homo sapiens
QSYDRGTHPALL

SEQ ID NO: 19 - protein, 17 aa, Homo sapiens
FIRYDGSNKYYADSVKG

SEQ ID NO: 20 - protein, 7 aa, Homo sapiens
GNDQRPS

SEQ ID NO: 21 - protein, 9 aa, Homo sapiens
FTFSSYGMH

SEQ ID NO: 22 - protein, 13 aa, Homo sapiens
SGGRSNIGSNTVK

SEQ ID NO: 23 - protein, 115 aa, Homo sapiens
QVQLVQSGGGVVQPGRSLRLSCAASGFTFSSYGMHWVRQAPGKGLEWVAFIRYDGSNKYYADSVKGRFTISRDNSKNTLYLQMKSLRAEDTAVYYCKTHGSHDNWGQGTMVTVSS

SEQ ID NO: 24 - protein, 112 aa, Homo sapiens
SYVLTQPPSVSGTPGQRVTISCSGGRSWIGSNTVKWYQQLPGTAPKLLIYGNDQRPSGVPDRFSGSKSGTSASLAITGVQAEDEADYYCQSYDRGTHPALLFGTGTKVTVLG

SEQ ID NO: 25 - protein, 6 aa, Homo sapiens
HGSHDN

SEQ ID NO: 26 - protein, 12 aa, Homo sapiens
QSYDRYTHPALL

SEQ ID NO: 27 - protein, 17 aa, Homo sapiens
FIRYDGSNKYYADSVKG

SEQ ID NO: 28 - protein, 7 aa, Homo sapiens
YNDQRPS

SEQ ID NO: 29 - protein, 9 aa, Homo sapiens
FTFSSYGMH

SEQ ID NO: 30 - protein, 13 aa, Homo sapiens
SGSRSNIGSNTVK

SEQ ID NO: 31 - protein, 115 aa, Homo sapiens [sequence omitted]

SEQ ID NO: 32 - protein, 112 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

SEQ ID NO: 33 - protein, 115 aa, Homo sapiens
QVQLVQSGGGVVQPGRSLRLSCAASGFTFSSYGMHWVRQAPGKGLEWVAFIRYDGSNKYYADSVKGRFTISRDNSKNTLYLQMKSLRAEDTAVYYCTTSGSYDYWGQGTMVTVSS

SEQ ID NO: 34 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDSSLRGSRVFGTGTKVTVLG

SEQ ID NO: 35 - protein, 115 aa, Homo sapiens
QVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAKSGSYDYWGQGTMVTVSS

SEQ ID NO: 36 - protein, 112 aa, Homo sapiens
  MOD_RES (32)..(32): Gly or Tyr
QSVLTQPPSVSGAPGQRVTISCTGSSSNIGAXDVHWYQQLPGTAPKLLIYGNSNRPSGVPDRFSGSKSGTSASLAITGLQAEDEADYYCQSYDSSLSGSRVFGTGTKVTVLG

SEQ ID NO: 37 - protein, 115 aa, Homo sapiens
QVQLVQSGGGVVQPGRSLRLSCAASGFTFSSYGMHWVRQAPGKGLEWVAFIRYDGSNKYYADSVKGRFTISRDNSKNTLYLQMKSLRAEDTAVYYCTTHGSHDNWGQGTMVTVSS

SEQ ID NO: 38 - protein, 112 aa, Homo sapiens; identical to SEQ ID NO: 34

SEQ ID NO: 39 - protein, 115 aa, Homo sapiens; identical to SEQ ID NO: 33

SEQ ID NO: 40 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDRGFTGSRVFGTGTKVTVLG

SEQ ID NO: 41 - protein, 115 aa, Homo sapiens; identical to SEQ ID NO: 33

SEQ ID NO: 42 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDSSLWGSRVFGTGTKVTVLG

SEQ ID NO: 43 - protein, 115 aa, Homo sapiens; identical to SEQ ID NO: 37

SEQ ID NO: 44 - protein, 112 aa, Homo sapiens; identical to SEQ ID NO: 40

SEQ ID NO: 45 - protein, 115 aa, Homo sapiens; identical to SEQ ID NO: 37

SEQ ID NO: 46 - protein, 112 aa, Homo sapiens; identical to SEQ ID NO: 42

SEQ ID NO: 47 - protein, 115 aa, Homo sapiens; identical to SEQ ID NO: 23

SEQ ID NO: 48 - protein, 112 aa, Homo sapiens
SYVLTQPPSVSGTPGQRVTISCSGSRSNIGSNTVKWYQQLPGTAPKLLIYGNDQRPSGVPDRFSGSKSGTSASLAITGVQAEDEADYYCQTYDKGFTGSSVFGTGTKVTVLG

SEQ ID NO: 49 - protein, 115 aa, Homo sapiens [sequence omitted]

SEQ ID NO: 50 - protein, 112 aa, Homo sapiens
QSVLTQPPSVSGAPGQRVTISCSGSRSNIGSNTVKWYQQLPGTAPKLLIYGNDQRPSGVPDRFSGSKSGTSASLAITGLQAEDEADYYCQTYDKGFTGSSVFGTGTKVTVLG

SEQ ID NO: 51 - protein, 115 aa, Homo sapiens
QVQLVQSGGGVVQPGRSLRLSCAASGFTFSSYGMHWVRQAPGKGLEWVAFIRYDGSNKYYADSVKGRFTISRDNSKNTLYLQMKSLRAEDTAVYYCTTHGSHDTWGQGTMVTVSS

SEQ ID NO: 52 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDSSLWGTRVFGTGTKVTVLG

SEQ ID NO: 53 - protein, 115 aa, Homo sapiens; identical to SEQ ID NO: 37

SEQ ID NO: 54 - protein, 112 aa, Homo sapiens
SYVLTQPPSVSGTPGQRVTISCSGGRSNIVSNTVKWYQQLPGTAPKLLIYGNDQRPSGVPDRFSGSKSGTSASLAITGVQAEDEADYYCQSYDRGFTGSRVFGTGTKVTVLG

SEQ ID NO: 55 - protein, 115 aa, Homo sapiens; identical to SEQ ID NO: 37

SEQ ID NO: 56 - protein, 112 aa, Homo sapiens
SYVLTQPPSVSGTPGQRVTISCSGGRSNIVSNTVKWYQQLPGTAPKLLIYGNDQRPSGVPDRFSGSKSGTSASLAITGVQAEDEADYYCQSYDRGFTGARVFGTGTKVTVLG

SEQ ID NO: 57 - protein, 115 aa, Homo sapiens; identical to SEQ ID NO: 23

SEQ ID NO: 58 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQTYDKGFTGSSVFGTGTKVTVLG

SEQ ID NO: 59 - protein, 115 aa, Homo sapiens; identical to SEQ ID NO: 23

SEQ ID NO: 60 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYERGFTGSMVFGTGTKVTVLG

SEQ ID NO: 61 - protein, 115 aa, Homo sapiens; identical to SEQ ID NO: 23

SEQ ID NO: 62 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDRGTHPLTIFGTGTKVTVLG

SEQ ID NO: 63 - protein, 115 aa, Homo sapiens; identical to SEQ ID NO: 23

SEQ ID NO: 64 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDRGSHPALTFGTGTKVTVLG

SEQ ID NO: 65 - protein, 115 aa, Homo sapiens; identical to SEQ ID NO: 23

SEQ ID NO: 66 - protein, 112 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDRGTHPLTMFGTGTKVTVLG

SEQ ID NO: 67 - protein, 115 aa, Homo sapiens [sequence omitted]

SEQ ID NO: 68 - protein, 112 aa, Homo sapiens
QSVLTQPPSVSGAPGQRVTISCSGSRSNIGSNTVKWYQQLPGTAPKLLIYGNDQRPSGVPDRFSGSKSGTSASLAITGLQAEDEADYYCQSYDRGTHPLTMFGTGTKVTVLG

SEQ ID NO: 69 - protein, 115 aa, Homo sapiens [sequence omitted]

SEQ ID NO: 70 - protein, 112 aa, Homo sapiens
QSVLTQPPSVSGAPGQRVTISCSGSRSNIGSNTVKWYQQLPGTAPKLLIYGNDQRPSGVPDRFSGSKSGTSASLAITGLQAEDEADYYCQSYDRGTHPALLFGTGTKVTVLG

SEQ ID NO: 71 - protein, 115 aa, Homo sapiens
QVQLVQSGGGVVQPGRSLRLSCAASGFTFSEYGMHWVRQAPGKGLEWVAFIRYDGSNKYYADSVKGRFTISRDNSKNTLYLQMKSLRAEDTAVYYCKTHGSHDNWGQGTMVTVSS

SEQ ID NO: 72 - protein, 112 aa, Homo sapiens; identical to SEQ ID NO: 70

SEQ ID NO: 73 - protein, 115 aa, Homo sapiens; identical to SEQ ID NO: 23

SEQ ID NO: 74 - protein, 112 aa, Homo sapiens
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRGTHPALLFGTGTKVTVLG

SEQ ID NO: 75 - protein, 115 aa, Homo sapiens; identical to SEQ ID NO: 23

SEQ ID NO: 76 - protein, 112 aa, Homo sapiens
QSVLTQPPSVSGAPGQRVTISCSGSRSNIGSNTVKWYQQLPGTAPKLLIYGNDQRPSGVPDRFSGSKSGTSASLAITGLQAEDEADYYCQSYDRYTHPALLFGTGTKVTVLG

SEQ ID NO: 77 - protein, 6 aa, Homo sapiens
SGSYDY

SEQ ID NO: 78 - protein, 6 aa, Homo sapiens
HGSHDN

SEQ ID NO: 79 - protein, 6 aa, Homo sapiens
HGSYDY

SEQ ID NO: 80 - protein, 6 aa, Homo sapiens
RRRSNY

SEQ ID NO: 81 - protein, 6 aa, Homo sapiens
SGSIDY

SEQ ID NO: 82 - protein, 6 aa, Homo sapiens
HGSHDD

SEQ ID NO: 83 - protein, 6 aa, Homo sapiens
HGSHDN

SEQ ID NO: 84 - protein, 12 aa, Homo sapiens
TTHGSHDNWGQG

SEQ ID NO: 85 - protein, 12 aa, Homo sapiens
AKHGSHDNWGQG

SEQ ID NO: 86 - protein, 12 aa, Homo sapiens
TTHGSHDNWSQG

SEQ ID NO: 87 - protein, 12 aa, Homo sapiens
TTHGSHDTWGQG

SEQ ID NO: 88 - protein, 12 aa, Homo sapiens
KTHGSHDNWGQG

SEQ ID NO: 89 - protein, 12 aa, Homo sapiens
KTHGSHDNWGHG

SEQ ID NO: 90 - protein, 12 aa, Homo sapiens
TTHGSHDNWSQG

SEQ ID NO: 91 - protein, 12 aa, Homo sapiens
TTHRSHNNWGQG

SEQ ID NO: 92 - protein, 8 aa, Homo sapiens
TTHGSHDN

SEQ ID NO: 93 - protein, 8 aa, Homo sapiens
TTHGSHDT

SEQ ID NO: 94 - protein, 8 aa, Homo sapiens
TKHGSHDN

SEQ ID NO: 95 - protein, 8 aa, Homo sapiens
TTQGRHDN

SEQ ID NO: 96 - protein, 8 aa, Homo sapiens
KTRGRHDN

SEQ ID NO: 97 - protein, 8 aa, Homo sapiens
TTHGSHDK

SEQ ID NO: 98 - protein, 8 aa, Homo sapiens
TTHGSHDD

SEQ ID NO: 99 - protein, 8 aa, Homo sapiens
KTHGSHDN

SEQ ID NO: 100 - protein, 8 aa, Homo sapiens
KTHGSHDN

SEQ ID NO: 101 - protein, 8 aa, Homo sapiens
TTHGSHDN

SEQ ID NO: 102 - protein, 8 aa, Homo sapiens
TTSGSYDY

SEQ ID NO: 103 - protein, 8 aa, Homo sapiens
TTHGSHDN

SEQ ID NO: 104 - protein, 8 aa, Homo sapiens
TTHGSQDN

SEQ ID NO: 105 - protein, 8 aa, Homo sapiens
ATHGSQDN

SEQ ID NO: 106 - protein, 6 aa, Homo sapiens
HGSQDT

SEQ ID NO: 107 - protein, 6 aa, Homo sapiens
SGSYDY

SEQ ID NO: 108 - protein, 6 aa, Homo sapiens
HGSQDN

SEQ ID NO: 109 - protein, 9 aa, Homo sapiens
CKTHGSHDN

SEQ ID NO: 110 - protein, 12 aa, Homo sapiens
QSYDSSLRGSRV

SEQ ID NO: 111 - protein, 12 aa, Homo sapiens
QSYDRGFTGSRV

SEQ ID NO: 112 - protein, 12 aa, Homo sapiens
QSYDSSLRGSRV

SEQ ID NO: 113 - protein, 12 aa, Homo sapiens
QSYDSSLTGSRV

SEQ ID NO: 114 - protein, 12 aa, Homo sapiens
QSYDSSLWGSRV

SEQ ID NO: 115 - protein, 12 aa, Homo sapiens
QTYDISESGSRV

SEQ ID NO: 116 - protein, 12 aa, Homo sapiens
QSYDRGFTGSRV

SEQ ID NO: 117 - protein, 12 aa, Homo sapiens
QTYDRGFTGSRV

SEQ ID NO: 118 - protein, 12 aa, Homo sapiens
QTYDKGFTGSSV

SEQ ID NO: 119 - protein, 12 aa, Homo sapiens
QSYDRRFTGSRV

SEQ ID NO: 120 - protein, 12 aa, Homo sapiens
QSYDWNFTGSRV

SEQ ID NO: 121 - protein, 12 aa, Homo sapiens
QSYDRGFTGSRV

SEQ ID NO: 122 - protein, 12 aa, Homo sapiens
QSYDNGFTGSRV

SEQ ID NO: 123 - protein, 12 aa, Homo sapiens
QSYDNAVTASKV

SEQ ID NO: 124 - protein, 12 aa, Homo sapiens
QSYDRGFTGSRV

SEQ ID NO: 125 - protein, 12 aa, Homo sapiens
QSYDSSLWGTRV

SEQ ID NO: 126 - protein, 12 aa, Homo sapiens
QSYDRDFTGSRV

SEQ ID NO: 127 - protein, 12 aa, Homo sapiens
QSYERGFTGSMV

SEQ ID NO: 128 - protein, 12 aa, Homo sapiens
QSYDNGFTGARV

SEQ ID NO: 129 - protein, 12 aa, Homo sapiens
QSYDRRFTGSRV

SEQ ID NO: 130 - protein, 12 aa, Homo sapiens
QTYDKGFTGSSV

SEQ ID NO: 131 - protein, 12 aa, Homo sapiens
QSYDRDFTGTRV

SEQ ID NO: 132 - protein, 12 aa, Homo sapiens
QSYDRGFYGSMV

SEQ ID NO: 133 - protein, 12 aa, Homo sapiens
QTYDKGFTGSSV

SEQ ID NO: 134 - protein, 12 aa, Homo sapiens
QSYDRGFTGARV

SEQ ID NO: 135 - protein, 12 aa, Homo sapiens
QSYERGFTGARV

SEQ ID NO: 136 - protein, 13 aa, Homo sapiens
QSYDRGFTGSRVF

SEQ ID NO: 137 - protein, 13 aa, Homo sapiens
QSYDRGFTGFKVF

SEQ ID NO: 138 - protein, 13 aa, Homo sapiens
QSYDRGFVSAYVF

SEQ ID NO: 139 - protein, 13 aa, Homo sapiens
QSYDRGLTVTKVF

SEQ ID NO: 140 - protein, 13 aa, Homo sapiens
QSYDRGYTASRVF

SEQ ID NO: 141 - protein, 13 aa, Homo sapiens
QSYDRGFTGSKVF

SEQ ID NO: 142 - protein, 13 aa, Homo sapiens
QSYDRGLTGFRVF

SEQ ID NO: 143 - protein, 13 aa, Homo sapiens
QSYDRGFTGYKVF

SEQ ID NO: 144 - protein, 13 aa, Homo sapiens
QSYDRGLTGYRLF

SEQ ID NO: 145 - protein, 13 aa, Homo sapiens
QSYDRGFTDYKVF

SEQ ID NO: 146 - protein, 13 aa, Homo sapiens
QSYDRGFTGPRLF

SEQ ID NO: 147 - protein, 13 aa, Homo sapiens
QSYDRGLTGSRVF

SEQ ID NO: 148 - protein, 13 aa, Homo sapiens
QSYDRGFTGARVW

SEQ ID NO: 149 - protein, 13 aa, Homo sapiens
QSYDRGFTGYRVF

SEQ ID NO: 150 - protein, 13 aa, Homo sapiens
QSYDRGFTGPRVF

SEQ ID NO: 151 - protein, 13 aa, Homo sapiens
QSYDRGMTSSRVF

SEQ ID NO: 152 - protein, 13 aa, Homo sapiens
QSYDRDSTGSRVF

SEQ ID NO: 153 - protein, 13 aa, Homo sapiens
QSYDSSLRGSRVF

SEQ ID NO: 154 - protein, 13 aa, Homo sapiens
HSYDSDFTGSRVF

SEQ ID NO: 155 - protein, 13 aa, Homo sapiens
HSSESGFTGSRVF

SEQ ID NO: 156 - protein, 13 aa, Homo sapiens
HSYDNRFTGSRVF

SEQ ID NO: 157 - protein, 13 aa, Homo sapiens
HSYDSRFTGSRVF

SEQ ID NO: 158 - protein, 13 aa, Homo sapiens
QSYDSEFTGSRVF

SEQ ID NO: 159 - protein, 13 aa, Homo sapiens
QSYDTGFTGSRVF

SEQ ID NO: 160 - protein, 13 aa, Homo sapiens
HSYDSGFTGSRVF

SEQ ID NO: 161 - protein, 13 aa, Homo sapiens
QSYDTGFTGSRVF

SEQ ID NO: 162 - protein, 13 aa, Homo sapiens
HSYDTKFTGSRVF

SEQ ID NO: 163 - protein, 13 aa, Homo sapiens
HSSDSGFTGSRVF

SEQ ID NO: 164 - protein, 13 aa, Homo sapiens
QSYDSDFTGSRVF

SEQ ID NO: 165 - protein, 13 aa, Homo sapiens
HSYESGFTGSRVF

SEQ ID NO: 166 - protein, 13 aa, Homo sapiens
QSYDAPWSGSRVF

SEQ ID NO: 167 - protein, 13 aa, Homo sapiens
QSYDSDFTGSKVF

SEQ ID NO: 168 - protein, 13 aa, Homo sapiens
HTNDSGFTGSRVF

SEQ ID NO: 169 - protein, 13 aa, Homo sapiens
HSYDTRFTGSRVF

SEQ ID NO: 170 - protein, 13 aa, Homo sapiens
QSYDMRFTGSRVF

SEQ ID NO: 171 - protein, 13 aa, Homo sapiens
HSSDSDSTGSRVF

SEQ ID NO: 172 - protein, 13 aa, Homo sapiens
QSYNTDFTGSRVF

SEQ ID NO: 173 - protein, 13 aa, Homo sapiens
QSYDSGFTGSRVF

SEQ ID NO: 174 - protein, 13 aa, Homo sapiens
HSYDMGFTGSRVF

SEQ ID NO: 175 - protein, 13 aa, Homo sapiens
HSYDNGFTGSRVF

SEQ ID NO: 176 - protein, 13 aa, Homo sapiens
HSHDRDFTGSRVF

SEQ ID NO: 177 - protein, 12 aa, Homo sapiens
QSYDSSLRGSRV

SEQ ID NO: 178 - protein, 13 aa, Homo sapiens
QSYDRGIHGSRVF

SEQ ID NO: 179 - protein, 13 aa, Homo sapiens
QSYDSGFPGSRVF

SEQ ID NO: 180 - protein, 13 aa, Homo sapiens
QSYDIGSTGSRVF

SEQ ID NO: 181 - protein, 13 aa, Homo sapiens
QSYDSGLTGSRVF

SEQ ID NO: 182 - protein, 13 aa, Homo sapiens
QSYDIGMTGSRVF

SEQ ID NO: 183 - protein, 13 aa, Homo sapiens
QSYDIGLTGSRVF

SEQ ID NO: 184 - protein, 13 aa, Homo sapiens
QSYDSGVTGSRVF

SEQ ID NO: 185 - protein, 13 aa, Homo sapiens
QSYDRGLTASRVF

SEQ ID NO: 186 - protein, 13 aa, Homo sapiens
QSYDTGLTGSRVF

SEQ ID NO: 187 - protein, 13 aa, Homo sapiens
QSYDTALTGSRVF

SEQ ID NO: 188 - protein, 13 aa, Homo sapiens
QSYDIRFTGSRVF

SEQ ID NO: 189 - protein, 13 aa, Homo sapiens
QSYDIRSTGSRVF

SEQ ID NO: 190 - protein, 13 aa, Homo sapiens
QSYDNRLTGSRVF

SEQ ID NO: 191 - protein, 13 aa, Homo sapiens
QSYETSFTGSRVF

SEQ ID NO: 192 - protein, 13 aa, Homo sapiens
QSYDSSSTGSRVF

SEQ ID NO: 193 - protein, 13 aa, Homo sapiens
QSYDSGFTASRVF

SEQ ID NO: 194 - protein, 13 aa, Homo sapiens
QTYDKGFTGSSVF

SEQ ID NO: 195 - protein, 13 aa, Homo sapiens
QSYDNGFTGSRVF

SEQ ID NO: 196 - protein, 13 aa, Homo sapiens
QSYDTGFTKSRVF

SEQ ID NO: 197 - protein, 13 aa, Homo sapiens
QSYDSDVTGSRVF

SEQ ID NO: 198 - protein, 13 aa, Homo sapiens
QSYDAGFTGSRVF

SEQ ID NO: 199 - protein, 12 aa, Homo sapiens
QSYDRGTHPSML

SEQ ID NO: 200 - protein, 12 aa, Homo sapiens
QSYDRGTTPRPM

SEQ ID NO: 201 - protein, 12 aa, Homo sapiens
QSYDRGRNPALT

SEQ ID NO: 202 - protein, 12 aa, Homo sapiens
QSYDRGTHPWLH

SEQ ID NO: 203 - protein, 12 aa, Homo sapiens
QSYDRGNSPATV

SEQ ID NO: 204 - protein, 12 aa, Homo sapiens
QSYDRGTFPSPQ

SEQ ID NO: 205 - protein, 12 aa, Homo sapiens
QSYDRGLNPSAT

SEQ ID NO: 206 - protein, 12 aa, Homo sapiens
QSYDRGKSNKML

SEQ ID NO: 207 - protein, 12 aa, Homo sapiens
QSYDRGHTAHLY

SEQ ID NO: 208 - protein, 12 aa, Homo sapiens
QSYDRGQTPSIT

SEQ ID NO: 209 - protein, 12 aa, Homo sapiens
QSYDRGYPRNIL

SEQ ID NO: 210 - protein, 12 aa, Homo sapiens
QSYDRGITPGLA

SEQ ID NO: 211 - protein, 12 aa, Homo sapiens
QSYDRGQPHAVL

SEQ ID NO: 212 - protein, 12 aa, Homo sapiens
QSYDRGNSPIPT

SEQ ID NO: 213 - protein, 12 aa, Homo sapiens
QSYDRGTPNNSF

SEQ ID NO: 214 - protein, 12 aa, Homo sapiens
QSYDSGVDPGPY

SEQ ID NO: 215 - protein, 12 aa, Homo sapiens
QSYDRGRPRHAL

SEQ ID NO: 216 - protein, 12 aa, Homo sapiens
QSYDRGPYHPIR

SEQ ID NO: 217 - protein, 12 aa, Homo sapiens
QSYDRGPHTQPT

SEQ ID NO: 218 - protein, 12 aa, Homo sapiens
QSYDRGHNNFSP

SEQ ID NO: 219 - protein, 12 aa, Homo sapiens
QSYDRGPTHLPH

SEQ ID NO: 220 - protein, 12 aa, Homo sapiens
QSYDRGTPSYPT

SEQ ID NO: 221 - protein, 12 aa, Homo sapiens
QSYDSGTSNLLP

SEQ ID NO: 222 - protein, 12 aa, Homo sapiens
QSYDRGDSNHDL

SEQ ID NO: 223 - protein, 12 aa, Homo sapiens
QSYDRGLPRLTH

SEQ ID NO: 224 - protein, 12 aa, Homo sapiens
QSYDRGIPTSYL

SEQ ID NO: 225 - protein, 12 aa, Homo sapiens
QSYDRGLRVQAP

SEQ ID NO: 226 - protein, 12 aa, Homo sapiens
QSYDRGLSDSPL

SEQ ID NO: 227 - protein, 12 aa, Homo sapiens
QSYDSGSLRRIL

SEQ ID NO: 228 - protein, 12 aa, Homo sapiens
QSYDRGPARTSP

SEQ ID NO: 229 - protein, 12 aa, Homo sapiens
QSYDRGRAAHPQ

SEQ ID NO: 230 - protein, 12 aa, Homo sapiens
QSYDRGTQPABI

SEQ ID NO: 231 - protein, 12 aa, Homo sapiens
QSYDRGTHPTMI

SEQ ID NO: 232 - protein, 12 aa, Homo sapiens
QSYDRGRIPABT

SEQ ID NO: 233 - protein, 12 aa, Homo sapiens
QSYDRGTHPVPA

SEQ ID NO: 234 - protein, 12 aa, Homo sapiens
QSYDRGSBPIPA

SEQ ID NO: 235 - protein, 12 aa, Homo sapiens
QSYDRGTHPVPA

SEQ ID NO: 236 - protein, 12 aa, Homo sapiens
QSYDRGTHPTMY

SEQ ID NO: 237 - protein, 12 aa, Homo sapiens
QSYDRGHHYTTF

SEQ ID NO: 238 - protein, 12 aa, Homo sapiens
QSYDRGSHPAAE

SEQ ID NO: 239 - protein, 12 aa, Homo sapiens
QSYDRGTIPSIE

SEQ ID NO: 240 - protein, 12 aa, Homo sapiens
QSYDRGSSPAIM

SEQ ID NO: 241 - protein, 12 aa, Homo sapiens
QSYDRGIWPNLN

SEQ ID NO: 242 - protein, 12 aa, Homo sapiens
QSYDRGTHPNLN

SEQ ID NO: 243 - protein, 12 aa, Homo sapiens
QSYDRGTHPSIS

SEQ ID NO: 244 - protein, 12 aa, Homo sapiens
QSYDRGSAPMIN

SEQ ID NO: 245 - protein, 12 aa, Homo sapiens
QSYDRGHHPAMS

SEQ ID NO: 246 - protein, 12 aa, Homo sapiens
QSYDRGTHPSIT

SEQ ID NO: 247 - protein, 12 aa, Homo sapiens
QSYDRGTDPAIV

SEQ ID NO: 248 - protein, 12 aa, Homo sapiens
QSYDRGTHPALL

SEQ ID NO: 249 - protein, 12 aa, Homo sapiens
QSYDRGSHPALT

SEQ ID NO: 250 - protein, 12 aa, Homo sapiens
QSYDRGTTPAPE

SEQ ID NO: 251 - protein, 12 aa, Homo sapiens
QSYDRGSHPTLI

SEQ ID NO: 252 - protein, 12 aa, Homo sapiens
QSYDRGTHPSML

SEQ ID NO: 253 - protein, 12 aa, Homo sapiens
QSYDRGTTPRPM

SEQ ID NO: 254 - protein, 12 aa, Homo sapiens
QSYDRGRLPAQT

SEQ ID NO: 255 - protein, 12 aa, Homo sapiens
QSYDRGTHPLTI

SEQ ID NO: 256 - protein, 12 aa, Homo sapiens
QSYDRGQTPSIT

SEQ ID NO: 257 - protein, 12 aa, Homo sapiens
QSYDRGTHFQMY

SEQ ID NO: 258 - protein, 12 aa, Homo sapiens
QSYDRGRNPALT

SEQ ID NO: 259 - protein, 12 aa, Homo sapiens
QSYDRGTHPLTM

SEQ ID NO: 260 - protein, 12 aa, Homo sapiens
QSYDRGTHPLTM

SEQ ID NO: 261 - protein, 12 aa, Homo sapiens
QSYDSGYTGSRV

SEQ ID NO: 262 - protein, 12 aa, Homo sapiens
QSYDSGFTGSRV

SEQ ID NO: 263 - protein, 12 aa, Homo sapiens
QSYDSRFTGSRV

SEQ ID NO: 264 - protein, 12 aa, Homo sapiens
QSYPDGTPASRV

SEQ ID NO: 265 - protein, 12 aa, Homo sapiens
QSYSTHMPISRV

SEQ ID NO: 266 - protein, 12 aa, Homo sapiens
QSYDSGSTGSRV

SEQ ID NO: 267 - protein, 12 aa, Homo sapiens
QSYPNSYPISRV

SEQ ID NO: 268 - protein, 10 aa, Homo sapiens
QSYIRAPQQV

SEQ ID NO: 269 - protein, 12 aa, Homo sapiens
QSYLKSRAFSRV

SEQ ID NO: 270 - protein, 12 aa, Homo sapiens
QSYDSRFTGSRV

SEQ ID NO: 271 - protein, 12 aa, Homo sapiens
QSYDRGFTGSMV

SEQ ID NO: 272 - protein, 12 aa, Homo sapiens
QSYDRGFTGSMV

SEQ ID NO: 273 - protein, 12 aa, Homo sapiens
QSYDRGFTGFDG

SEQ ID NO: 274 - protein, 12 aa, Homo sapiens
QSYDRGTAPALS

SEQ ID NO: 275 - protein, 12 aa, Homo sapiens
QSYDRGSYPALR

SEQ ID NO: 276 - protein, 12 aa, Homo sapiens
QSYDRGNWPNSN

SEQ ID NO: 277 - protein, 12 aa, Homo sapiens
QSYDRGTAPSLL

SEQ ID NO: 278 - protein, 12 aa, Homo sapiens
QSYDRGFTGSMV

SEQ ID NO: 279 - protein, 12 aa, Homo sapiens
QSYDRGTTPRIR

SEQ ID NO: 280 - protein, 12 aa, Homo sapiens
QSYDRGFTGSMV

SEQ ID NO: 281 - protein, 12 aa, Homo sapiens
QSYDRGFTGSMV

SEQ ID NO: 282 - protein, 12 aa, Homo sapiens
QSYDRGMIPALT

SEQ ID NO: 283 - protein, 12 aa, Homo sapiens
QSYDRNTHPALL

SEQ ID NO: 284 - protein, 12 aa, Homo sapiens
QSYDRFTHPALL

SEQ ID NO: 285 - protein, 12 aa, Homo sapiens
QSYDRYTHPALL

SEQ ID NO: 286 - protein, 12 aa, Homo sapiens
QSYDRGTHPALL

SEQ ID NO: 287 - protein, 12 aa, Homo sapiens
QSYDRYTHPALL

SEQ ID NO: 288 - protein, 9 aa, Homo sapiens
FTFESYGMH

SEQ ID NO: 289 - protein, 9 aa, Homo sapiens
FTFSSYGMH

SEQ ID NO: 290 - protein, 9 aa, Homo sapiens
FTFYSYGMH

SEQ ID NO: 291 - protein, 9 aa, Homo sapiens
FTFHSYGMH

SEQ ID NO: 292 - protein, 9 aa, Homo sapiens
FTFKSYGMH

SEQ ID NO: 293 - protein, 9 aa, Homo sapiens
FTFRSYGMH

SEQ ID NO: 294 - protein, 9 aa, Homo sapiens
FTFNSYGMH

SEQ ID NO: 295 - protein, 9 aa, Homo sapiens
FTFTSYGMH

SEQ ID NO: 296 - protein, 9 aa, Homo sapiens
FTFGSYGMH

SEQ ID NO: 297 - protein, 9 aa, Homo sapiens
FTFVSYGMH

SEQ ID NO: 298 - protein, 9 aa, Homo sapiens
FTFISYGMH

SEQ ID NO: 299 - protein, 9 aa, Homo sapiens
FTFWSYGMH

SEQ ID NO: 300 - protein, 9 aa, Homo sapiens
FTFSEYGMH

SEQ ID NO: 301 - protein, 9 aa, Homo sapiens
FTFSCYGMH

SEQ ID NO: 302 - protein, 9 aa, Homo sapiens
FTFSSYGMH

SEQ ID NO: 303 - protein, 9 aa, Homo sapiens
FTFSYYGMH

SEQ ID NO: 304 - protein, 9 aa, Homo sapiens
FTFSHYGMH

SEQ ID NO: 305 - protein, 9 aa, Homo sapiens
FTFSRYGMH

SEQ ID NO: 306 - protein, 9 aa, Homo sapiens
FTFSNYGMH

SEQ ID NO: 307 - protein, 9 aa, Homo sapiens
FTFSQYGMH

SEQ ID NO: 308 - protein, 9 aa, Homo sapiens
FTFSTYGMH

SEQ ID NO: 309 - protein, 9 aa, Homo sapiens
FTFSAYGMH

SEQ ID NO: 310 - protein, 9 aa, Homo sapiens
FTFSIYGMH

SEQ ID NO: 311 - protein, 9 aa, Homo sapiens
FTFSSEGMH

SEQ ID NO: 312 - protein, 9 aa, Homo sapiens
FTFSSCGMH

SEQ ID NO: 313 - protein, 9 aa, Homo sapiens
FTFSSSGMH

SEQ ID NO: 314 - protein, 9 aa, Homo sapiens
FTFSSYGMH

SEQ ID NO: 315 - protein, 9 aa, Homo sapiens
FTFSSHGMH

SEQ ID NO: 316 - protein, 9 aa, Homo sapiens
FTFSSRGMH

SEQ ID NO: 317 - protein, 9 aa, Homo sapiens
FTFSSNGMH

SEQ ID NO: 318 - protein, 9 aa, Homo sapiens
FTFSSTGMH

SEQ ID NO: 319 - protein, 9 aa, Homo sapiens
FTFSSAGMH

SEQ ID NO: 320 - protein, 9 aa, Homo sapiens
FTFSSVGMH

SEQ ID NO: 321 - protein, 9 aa, Homo sapiens
FTFSSLGMH

SEQ ID NO: 322 - protein, 9 aa, Homo sapiens
FTFSSIGMH

SEQ ID NO: 323 - protein, 9 aa, Homo sapiens
FTFSSYDMH

SEQ ID NO: 324 - protein, 9 aa, Homo sapiens
FTFSSYEMH

SEQ ID NO: 325 - protein, 9 aa, Homo sapiens
FTFSSYCMH

SEQ ID NO: 326 - protein, 9 aa, Homo sapiens
FTFSSYSMH

SEQ ID NO: 327 - protein, 9 aa, Homo sapiens
FTFSSYYMH

SEQ ID NO: 328 - protein, 9 aa, Homo sapiens
FTFSSYNMH

SEQ ID NO: 329 - protein, 9 aa, Homo sapiens
FTFSSYGMH

SEQ ID NO: 330 - protein, 9 aa, Homo sapiens
FTFSSYAMH

SEQ ID NO: 331 - protein, 9 aa, Homo sapiens
FTFSSYVMH

SEQ ID NO: 332 - protein, 9 aa, Homo sapiens
FTFSSYMMH

SEQ ID NO: 333 - protein, 9 aa, Homo sapiens
FTFSSYIMH

SEQ ID NO: 334 - protein, 9 aa, Homo sapiens
FTFSSYPMH

SEQ ID NO: 335 - protein, 17 aa, Homo sapiens
EIRYDGSNKYYADSVKG

SEQ ID NO: 336 - protein, 17 aa, Homo sapiens
CIRYDGSNKYYADSVKG

SEQ ID NO: 337 - protein, 17 aa, Homo sapiens
YIRYDGSNKYYADSVKG

SEQ ID NO: 338 - protein, 17 aa, Homo sapiens
HIRYDGSNKYYADSVKG

SEQ ID NO: 339 - protein, 17 aa, Homo sapiens
KIRYDGSNKYYADSVKG

SEQ ID NO: 340 - protein, 17 aa, Homo sapiens
NIRYDGSNKYYADSVKG

SEQ ID NO: 341 - protein, 17 aa, Homo sapiens
QIRYDGSNKYYADSVKG

SEQ ID NO: 342 - protein, 17 aa, Homo sapiens
TIRYDGSNKYYADSVKG

SEQ ID NO: 343 - protein, 17 aa, Homo sapiens
LIRYDGSNKYYADSVKG

SEQ ID NO: 344 - protein, 17 aa, Homo sapiens
FIRYDGSNKYYADSVKG

SEQ ID NO: 345 - protein, 17 aa, Homo sapiens
FIEYDGSNKYYADSVKG

SEQ ID NO: 346 - protein, 17 aa, Homo sapiens
FISYDGSNKYYADSVKG

SEQ ID NO: 347 - protein, 17 aa, Homo sapiens
FIYYDGSNKYYADSVKG

SEQ ID NO: 348 - protein, 17 aa, Homo sapiens
FIHYDGSNKYYADSVKG

SEQ ID NO: 349 - protein, 17 aa, Homo sapiens
FIKYDGSNKYYADSVKG

SEQ ID NO: 350 - protein, 17 aa, Homo sapiens
FIRYDGSNKYYADSVKG

SEQ ID NO: 351 - protein, 17 aa, Homo sapiens
FIQYDGSNKYYADSVKG

SEQ ID NO: 352 - protein, 17 aa, Homo sapiens
FITYDGSNKYYADSVKG

SEQ ID NO: 353 - protein, 17 aa, Homo sapiens
FIGYDGSNKYYADSVKG

SEQ ID NO: 354 - protein, 17 aa, Homo sapiens
FIAYDGSNKYYADSVKG

SEQ ID NO: 355 - protein, 17 aa, Homo sapiens
FIVYDGSNKYYADSVKG

SEQ ID NO: 356 - protein, 17 aa, Homo sapiens
FILYDGSNKYYADSVKG

SEQ ID NO: 357 - protein, 17 aa, Homo sapiens
FIWYDGSNKYYADSVKG

SEQ ID NO: 358 - protein, 17 aa, Homo sapiens
FIRYDGSNKYYADSVKG

SEQ ID NO: 359 - protein, 17 aa, Homo sapiens
FIRYEGSNKYYADSVKG

SEQ ID NO: 360 - protein, 17 aa, Homo sapiens
FIRYSGSNKYYADSVKG

SEQ ID NO: 361 - protein, 17 aa, Homo sapiens
FIRYYGSNKYYADSVKG

SEQ ID NO: 362 - protein, 17 aa, Homo sapiens
FIRYKGSNKYYADSVKG

SEQ ID NO: 363 - protein, 17 aa, Homo sapiens
FIRYRGSNKYYADSVKG

SEQ ID NO: 364 - protein, 17 aa, Homo sapiens
FIRYNGSNKYYADSVKG

SEQ ID NO: 365 - protein, 17 aa, Homo sapiens
FIRYQGSNKYYADSVKG

SEQ ID NO: 366 - protein, 17 aa, Homo sapiens
FIRYTGSNKYYADSVKG

SEQ ID NO: 367 - protein, 17 aa, Homo sapiens
FIRYAGSNKYYADSVKG

SEQ ID NO: 368 - protein, 17 aa, Homo sapiens
FIRYVGSNKYYADSVKG

SEQ ID NO: 369 - protein, 17 aa, Homo sapiens
FIRYLGSNKYYADSVKG

SEQ ID NO: 370 - protein, 17 aa, Homo sapiens
FIRYIGSNKYYADSVKG

SEQ ID NO: 371 - protein, 17 aa, Homo sapiens
FIRYFGSNKYYADSVKG

SEQ ID NO: 372 - protein, 17 aa, Homo sapiens
FIRYDDSNKYYADSVKG

SEQ ID NO: 373 - protein, 17 aa, Homo sapiens
FIRYDESNKYYADSVKG

SEQ ID NO: 374 - protein, 17 aa, Homo sapiens
FIRYDSSNKYYADSVKG

SEQ ID NO: 375 - protein, 17 aa, Homo sapiens
FIRYDYSNKYYADSVKG

SEQ ID NO: 376 - protein, 17 aa, Homo sapiens
FIRYDKSNKYYADSVKG

SEQ ID NO: 377 - protein, 17 aa, Homo sapiens
FIRYDRSNKYYADSVKG

SEQ ID NO: 378 - protein, 17 aa, Homo sapiens
FIRYDNSNKYYADSVKG

SEQ ID NO: 379 - protein, 17 aa, Homo sapiens
FIRYDQSNKYYADSVKG

SEQ ID NO: 380 - protein, 17 aa, Homo sapiens
FIRYDTSNKYYADSVKG

SEQ ID NO: 381 - protein, 17 aa, Homo sapiens
FIRYDGSNKYYADSVKG

SEQ ID NO: 382 - protein, 17 aa, Homo sapiens
FIRYDVSNKYYADSVKG

SEQ ID NO: 383 - protein, 17 aa, Homo sapiens
FIRYDFSNKYYADSVKG

SEQ ID NO: 384 - protein, 17 aa, Homo sapiens
FIRYDGSSKYYADSVKG

SEQ ID NO: 385 - protein, 17 aa, Homo sapiens
FIRYDGSYKYYADSVKG

SEQ ID NO: 386 - protein, 17 aa, Homo sapiens
FIRYDGSHKYYADSVKG

SEQ ID NO: 387 - protein, 17 aa, Homo sapiens
FIRYDGSNKYYADSVKG

SEQ ID NO: 388 - protein, 17 aa, Homo sapiens
FIRYDGSTKYYADSVKG

SEQ ID NO: 389 - protein, 17 aa, Homo sapiens
FIRYDGSGKYYADSVKG

SEQ ID NO: 390 - protein, 17 aa, Homo sapiens
FIRYDGSMKYYADSVKG

SEQ ID NO: 391 - protein, 17 aa, Homo sapiens
FIRYDGSLKYYADSVKG

SEQ ID NO: 392 - protein, 17 aa, Homo sapiens
FIRYDGSIKYYADSVKG

SEQ ID NO: 393 - protein, 17 aa, Homo sapiens
FIRYDGSPKYYADSVKG

SEQ ID NO: 394 - protein, 17 aa, Homo sapiens
FIRYDGSFKYYADSVKG

SEQ ID NO: 395 - protein, 17 aa, Homo sapiens
FIRYDGSNKEYADSVKG

SEQ ID NO: 396 - protein, 17 aa, Homo sapiens
FIRYDGSNKSYADSVKG

SEQ ID NO: 397 - protein, 17 aa, Homo sapiens
FIRYDGSNKYYADSVKG

SEQ ID NO: 398 - protein, 17 aa, Homo sapiens
FIRYDGSNKNYADSVKG

SEQ ID NO: 399 - protein, 17 aa, Homo sapiens
FIRYDGSNKVYADSVKG

SEQ ID NO: 400 - protein, 17 aa, Homo sapiens
FIRYDGSNKLYADSVKG

SEQ ID NO: 401 - protein, 17 aa, Homo sapiens
FIRYDGSNKIYADSVKG

SEQ ID NO: 402 - protein, 17 aa, Homo sapiens [sequence omitted]

SEQ ID NO: 403 - protein, 17 aa, Homo sapiens
FIRYDGSNKFYADSVKG

SEQ ID NO: 404 - protein, 6 aa, Homo sapiens
EGSHDN

SEQ ID NO: 405 - protein, 6 aa, Homo sapiens
SGSHDN

SEQ ID NO: 406 - protein, 6 aa, Homo sapiens
HGSHDN

SEQ ID NO: 407 - protein, 6 aa, Homo sapiens
KGSHDN

SEQ ID NO: 408 - protein, 6 aa, Homo sapiens
QGSHDN

SEQ ID NO: 409 - protein, 6 aa, Homo sapiens
TGSHDN

SEQ ID NO: 410 - protein, 6 aa, Homo sapiens
AGSHDN

SEQ ID NO: 411 - protein, 6 aa, Homo sapiens
LGSHDN

SEQ ID NO: 412 - protein, 6 aa, Homo sapiens
PGSHDN

SEQ ID NO: 413 - protein, 6 aa, Homo sapiens
FGSHDN

SEQ ID NO: 414 - protein, 6 aa, Homo sapiens
HDSHDN

SEQ ID NO: 415 - protein, 6 aa, Homo sapiens
HCSHDN

SEQ ID NO: 416 - protein, 6 aa, Homo sapiens
HHSHDN

SEQ ID NO: 417 - protein, 6 aa, Homo sapiens
HRSHDN

SEQ ID NO: 418 - protein, 6 aa, Homo sapiens
HTSHDN

SEQ ID NO: 419 - protein, 6 aa, Homo sapiens
HGSHDN

SEQ ID NO: 420 - protein, 6 aa, Homo sapiens
HVSHDN

SEQ ID NO: 421 - protein, 6 aa, Homo sapiens
HMSHDN

SEQ ID NO: 422 - protein, 6 aa, Homo sapiens
HLSHDN

SEQ ID NO: 423 - protein, 6 aa, Homo sapiens
HISHDN

SEQ ID NO: 424 - protein, 6 aa, Homo sapiens
HPSHDN

SEQ ID NO: 425 - protein, 6 aa, Homo sapiens
HWSHDN

SEQ ID NO: 426 - protein, 6 aa, Homo sapiens
HGDHDN

SEQ ID NO: 427 - protein, 6 aa, Homo sapiens
HGSHDN

SEQ ID NO: 428 - protein, 6 aa, Homo sapiens
HGYHDN

SEQ ID NO: 429 - protein, 6 aa, Homo sapiens
HGHHDN

SEQ ID NO: 430 - protein, 6 aa, Homo sapiens
HGRHDN

SEQ ID NO: 431 - protein, 6 aa, Homo sapiens
HGNHDN

SEQ ID NO: 432 - protein, 6 aa, Homo sapiens
HGTHDN

SEQ ID NO: 433 - protein, 6 aa, Homo sapiens
HGGHDN

SEQ ID NO: 434 - protein, 6 aa, Homo sapiens
HGAHDN

SEQ ID NO: 435 - protein, 6 aa, Homo sapiens
HGIHDN

SEQ ID NO: 436 - protein, 6 aa, Homo sapiens
HGPHDN

SEQ ID NO: 437 - protein, 6 aa, Homo sapiens
HGWHDN

SEQ ID NO: 438 - protein, 6 aa, Homo sapiens
HGFHDN

SEQ ID NO: 439 - protein, 6 aa, Homo sapiens
HGSHDN

SEQ ID NO: 440 - protein, 6 aa, Homo sapiens
HGSRDN

SEQ ID NO: 441 - protein, 6 aa, Homo sapiens
HGSTDN

SEQ ID NO: 442 - protein, 6 aa, Homo sapiens
HGSADN

SEQ ID NO: 443 - protein, 6 aa, Homo sapiens
HGSVDN

SEQ ID NO: 444 - protein, 6 aa, Homo sapiens
HGSLDN

SEQ ID NO: 445 - protein, 6 aa, Homo sapiens
HGSIDN

SEQ ID NO: 446 - protein, 6 aa, Homo sapiens
HGSFDN

SEQ ID NO: 447 - protein, 6 aa, Homo sapiens
HGSHDN

SEQ ID NO: 448 - protein, 6 aa, Homo sapiens
HGSHSN

SEQ ID NO: 449 - protein, 6 aa, Homo sapiens
HGSHYN

SEQ ID NO: 450 - protein, 6 aa, Homo sapiens
HGSHHN

SEQ ID NO: 451 - protein, 6 aa, Homo sapiens
HGSHRN

SEQ ID NO: 452 - protein, 6 aa, Homo sapiens
HGSHNN

SEQ ID NO: 453 - protein, 6 aa, Homo sapiens
HGSHGN

SEQ ID NO: 454 - protein, 6 aa, Homo sapiens
HGSHAN

SEQ ID NO: 455 - protein, 6 aa, Homo sapiens
HGSHVN

SEQ ID NO: 456 - protein, 6 aa, Homo sapiens
HGSHIN

SEQ ID NO: 457 - protein, 6 aa, Homo sapiens
HGSHDS

SEQ ID NO: 458 - protein, 6 aa, Homo sapiens
HGSHDH

SEQ ID NO: 459 - protein, 6 aa, Homo sapiens
HGSHDK

SEQ ID NO: 460 - protein, 6 aa, Homo sapiens
HGSHDR

SEQ ID NO: 461 - protein, 6 aa, Homo sapiens
HGSHDN

SEQ ID NO: 462 - protein, 6 aa, Homo sapiens
HGSHDT

SEQ ID NO: 463 - protein, 6 aa, Homo sapiens
HGSHDG

SEQ ID NO: 464 - protein, 6 aa, Homo sapiens
HGSHDA

SEQ ID NO: 465 - protein, 6 aa, Homo sapiens
HGSHDL

SEQ ID NO: 466 - protein, 6 aa, Homo sapiens
HGSHDI

SEQ ID NO: 467 - protein, 6 aa, Homo sapiens
HGSHDP

SEQ ID NO: 468 - protein, 6 aa, Homo sapiens
HGSHDW

SEQ ID NO: 469 - protein, 6 aa, Homo sapiens
HGSHDF

SEQ ID NO: 470 - protein, 13 aa, Homo sapiens
SGGRSNIGDNTVK

SEQ ID NO: 471 - protein, 13 aa, Homo sapiens
SGGRSNIGCNTVK

SEQ ID NO: 472 - protein, 13 aa, Homo sapiens
SGGRSNIGSNTVK

SEQ ID NO: 473 - protein, 13 aa, Homo sapiens
SGGRSNIGYNTVK

SEQ ID NO: 474 - protein, 13 aa, Homo sapiens
SGGRSNIGKNTVK

SEQ ID NO: 475 - protein, 13 aa, Homo sapiens
SGGRSNIGRNTVK

SEQ ID NO: 476 - protein, 13 aa, Homo sapiens
SGGRSNIGNNTVK

SEQ ID NO: 477 - protein, 13 aa, Homo sapiens
SGGRSNIGTNTVK

SEQ ID NO: 478 - protein, 13 aa, Homo sapiens
SGGRSNIGPNTVK

SEQ ID NO: 479 - protein, 13 aa, Homo sapiens
SGGRSNIGSDTVK

SEQ ID NO: 480 - protein, 13 aa, Homo sapiens
SGGRSNIGSETVK

SEQ ID NO: 481 - protein, 13 aa, Homo sapiens
SGGRSNIGSSTVK

SEQ ID NO: 482 - protein, 13 aa, Homo sapiens
SGGRSNIGSYTVK

SEQ ID NO: 483 - protein, 13 aa, Homo sapiens
SGGRSNIGSHTVK

SEQ ID NO: 484 - protein, 13 aa, Homo sapiens
SGGRSNIGSKTVK

SEQ ID NO: 485 - protein, 13 aa, Homo sapiens
SGGRSNIGSNTVK

SEQ ID NO: 486 - protein, 13 aa, Homo sapiens
SGGRSNIGSQTVK

SEQ ID NO: 487 - protein, 13 aa, Homo sapiens
SGGRSNIGSTTVK

SEQ ID NO: 488 - protein, 13 aa, Homo sapiens
SGGRSNIGSGTVK

SEQ ID NO: 489 - protein, 13 aa, Homo sapiens
SGGRSNIGSMTVK

SEQ ID NO: 490 - protein, 13 aa, Homo sapiens
SGGRSNIGSITVK

SEQ ID NO: 491 - protein, 13 aa, Homo sapiens
SGGRSNIGSNDVK

SEQ ID NO: 492 - protein, 13 aa, Homo sapiens
SGGRSNIGSNCVK

SEQ ID NO: 493 - protein, 13 aa, Homo sapiens
SGGRSNIGSNSVK

SEQ ID NO: 494 - protein, 13 aa, Homo sapiens
SGGRSNIGSNYVK

SEQ ID NO: 495 - protein, 13 aa, Homo sapiens
SGGRSNIGSNHVK

SEQ ID NO: 496 - protein, 13 aa, Homo sapiens
SGGRSNIGSNKVK

SEQ ID NO: 497 - protein, 13 aa, Homo sapiens
SGGRSNIGSNRVK

SEQ ID NO: 498 - protein, 13 aa, Homo sapiens
SGGRSNIGSNNVK

SEQ ID NO: 499 - protein, 13 aa, Homo sapiens
SGGRSNIGSNQVK

SEQ ID NO: 500 - protein, 13 aa, Homo sapiens
SGGRSNIGSNTVK

SEQ ID NO: 501 - protein, 13 aa, Homo sapiens
SGGRSNIGSNAVK

SEQ ID NO: 502 - protein, 13 aa, Homo sapiens
SGGRSNIGSNVVK

SEQ ID NO: 503 - protein, 13 aa, Homo sapiens
SGGRSNIGSNLVK

SEQ ID NO: 504 - protein, 13 aa, Homo sapiens
SGGRSNIGSNIVK

SEQ ID NO: 505 - protein, 13 aa, Homo sapiens
SGGRSNIGSNPVK

SEQ ID NO: 506 - protein, 7 aa, Homo sapiens
DNDQRPS

SEQ ID NO: 507 - protein, 7 aa, Homo sapiens
ENDQRPS

SEQ ID NO: 508 - protein, 7 aa, Homo sapiens
CNDQRPS

SEQ ID NO: 509 - protein, 7 aa, Homo sapiens
SNDQRPS

SEQ ID NO: 510 - protein, 7 aa, Homo sapiens
YNDQRPS

SEQ ID NO: 511 - protein, 7 aa, Homo sapiens
HNDQRPS

SEQ ID NO: 512 - protein, 7 aa, Homo sapiens
KNDQRPS

SEQ ID NO: 513 - protein, 7 aa, Homo sapiens
RNDQRPS

SEQ ID NO: 514 - protein, 7 aa, Homo sapiens
NNDQRPS

SEQ ID NO: 515 - protein, 7 aa, Homo sapiens
QNDQRPS

SEQ ID NO: 516 - protein, 7 aa, Homo sapiens
TNDQRPS

SEQ ID NO: 517 - protein, 7 aa, Homo sapiens
GNDQRPS

SEQ ID NO: 518 - protein, 7 aa, Homo sapiens
ANDQRPS

SEQ ID NO: 519 - protein, 7 aa, Homo sapiens
VNDQRPS

SEQ ID NO: 520 - protein, 7 aa, Homo sapiens
MNDQRPS

SEQ ID NO: 521 - protein, 7 aa, Homo sapiens
LNDQRPS

SEQ ID NO: 522 - protein, 7 aa, Homo sapiens
INDQRPS

SEQ ID NO: 523 - protein, 7 aa, Homo sapiens
PNDQRPS

SEQ ID NO: 524 - protein, 7 aa, Homo sapiens
WNDQRPS

SEQ ID NO: 525 - protein, 7 aa, Homo sapiens
FNDQRPS

SEQ ID NO: 526 - protein, 7 aa, Homo sapiens
GNDSRPS

SEQ ID NO: 527 - protein, 7 aa, Homo sapiens
GNDYRPS

SEQ ID NO: 528 - protein, 7 aa, Homo sapiens
GNDRRPS

SEQ ID NO: 529 - protein, 7 aa, Homo sapiens
GNDQRPS

SEQ ID NO: 530 - protein, 7 aa, Homo sapiens
GNDTRPS

SEQ ID NO: 531 - protein, 7 aa, Homo sapiens
GNDARPS

SEQ ID NO: 532 - protein, 7 aa, Homo sapiens
GNDIRPS

SEQ ID NO: 533 - protein, 7 aa, Homo sapiens
GNDPRPS

SEQ ID NO: 534 - protein, 12 aa, Homo sapiens
QSYDRGTHPALL

SEQ ID NO: 535 - protein, 12 aa, Homo sapiens
QSYCRGTHPALL

SEQ ID NO: 536 - protein, 12 aa, Homo sapiens
QSYSRGTHPALL

SEQ ID NO: 537 - protein, 12 aa, Homo sapiens
QSYYRGTHPALL

SEQ ID NO: 538 - protein, 12 aa, Homo sapiens
QSYNRGTHPALL

SEQ ID NO: 539 - protein, 12 aa, Homo sapiens
QSYQRGTHPALL

SEQ ID NO: 540 - protein, 12 aa, Homo sapiens
QSYTRGTHPALL

SEQ ID NO: 541 - protein, 12 aa, Homo sapiens
QSYGRGTHPALL

SEQ ID NO: 542 - protein, 12 aa, Homo sapiens
QSYARGTHPALL

SEQ ID NO: 543 - protein, 12 aa, Homo sapiens
QSYLRGTHPALL

SEQ ID NO: 544 - protein, 12 aa, Homo sapiens
QSYIRGTHPALL

SEQ ID NO: 545 - protein, 12 aa, Homo sapiens
QSYWRGTHPALL

SEQ ID NO: 546 - protein, 12 aa, Homo sapiens
QSYFRGTHPALL

SEQ ID NO: 547 - protein, 12 aa, Homo sapiens
QSYDDGTHPALL

SEQ ID NO: 548 - protein, 12 aa, Homo sapiens
QSYDCGTHPALL

SEQ ID NO: 549 - protein, 12 aa, Homo sapiens
QSYDSGTHPALL

SEQ ID NO: 550 - protein, 12 aa, Homo sapiens
QSYDYGTHPALL

SEQ ID NO: 551 - protein, 12 aa, Homo sapiens
QSYDRGTHPALL

SEQ ID NO: 552 - protein, 12 aa, Homo sapiens
QSYDNGTHPALL

SEQ ID NO: 553 - protein, 12 aa, Homo sapiens
QSYDQGTHPALL

SEQ ID NO: 554 - protein, 12 aa, Homo sapiens
QSYDTGTHPALL

SEQ ID NO: 555 - protein, 12 aa, Homo sapiens
QSYDGGTHPALL

SEQ ID NO: 556 - protein, 12 aa, Homo sapiens
QSYDAGTHPALL

SEQ ID NO: 557 - protein, 12 aa, Homo sapiens
QSYDVGTHPALL

SEQ ID NO: 558 - protein, 12 aa, Homo sapiens
QSYDMGTHPALL

SEQ ID NO: 559 - protein, 12 aa, Homo sapiens
QSYDLGTHPALL

SEQ ID NO: 560 - protein, 12 aa, Homo sapiens
QSYDIGTHPALL

SEQ ID NO: 561 - protein, 12 aa, Homo sapiens
QSYDPGTHPALL

SEQ ID NO: 562 - protein, 12 aa, Homo sapiens
QSYDWGTHPALL

SEQ ID NO: 563 - protein, 12 aa, Homo sapiens
QSYDRDTHPALL

SEQ ID NO: 564 - protein, 12 aa, Homo sapiens
QSYDRCTHPALL

SEQ ID NO: 565 - protein, 12 aa, Homo sapiens
QSYDRSTHPALL

SEQ ID NO: 566 - protein, 12 aa, Homo sapiens
QSYDRYTHPALL

SEQ ID NO: 567 - protein, 12 aa, Homo sapiens
QSYDRHTHPALL

SEQ ID NO: 568 - protein, 12 aa, Homo sapiens
QSYDRRTHPALL

SEQ ID NO: 569 - protein, 12 aa, Homo sapiens
QSYDRNTHPALL

SEQ ID NO: 570 - protein, 12 aa, Homo sapiens
QSYDRQTHPALL

SEQ ID NO: 571 - protein, 12 aa, Homo sapiens
QSYDRTTHPALL

SEQ ID NO: 572 - protein, 12 aa, Homo sapiens
QSYDRGTHPALL

SEQ ID NO: 573 - protein, 12 aa, Homo sapiens
QSYDRATHPALL

SEQ ID NO: 574 - protein, 12 aa, Homo sapiens
QSYDRVTHPALL

SEQ ID NO: 575 - protein, 12 aa, Homo sapiens
QSYDRLTHPALL

SEQ ID NO: 576 - protein, 12 aa, Homo sapiens
QSYDRITHPALL

SEQ ID NO: 577 - protein, 12 aa, Homo sapiens
QSYDRPTHPALL

SEQ ID NO: 578 - protein, 12 aa, Homo sapiens
QSYDRWTHPALL

SEQ ID NO: 579 - protein, 12 aa, Homo sapiens
QSYDRFTHPALL

SEQ ID NO: 580 - DNA, 48 bp, Artificial Sequence
  Description of Artificial Sequence Synthetic oligonucleotide
tgtcccttggccccannnnnnnnnnnnnnnnnnngtcgtacagtaata

SEQ ID NO: 581 - DNA, 35 bp, Artificial Sequence
  Description of Artificial Sequence Synthetic oligonucleotide
gacacctcgatcagcggataacaatttcacacagg

SEQ ID NO: 582 - DNA, 15 bp, Artificial Sequence
  Description of Artificial Sequence Synthetic oligonucleotide
tggggccaagggaca

SEQ ID NO: 583 - DNA, 45 bp, Artificial Sequence
  Description of Artificial Sequence Synthetic oligonucleotide
attcgtcctataccgttctactttgtcgtctttccagacgttagt

SEQ ID NO: 584 - DNA, 18 bp, Artificial Sequence
  Description of Artificial Sequence Synthetic oligonucleotide
attcgtcctataccgttc

SEQ ID NO: 585 - DNA, 66 bp, Artificial Sequence
  Description of Artificial Sequence Synthetic oligonucleotide
ggtcccagttccgaagaccctcgaaccnnnnnnnnnnnnnnnatatgactggcagtaatagtcagc

SEQ ID NO: 586 - DNA, 15 bp, Artificial Sequence
  Description of Artificial Sequence Synthetic oligonucleotide
tggggccaagggaca

SEQ ID NO: 587 - DNA, 24 bp, Artificial Sequence
  Description of Artificial Sequence Synthetic oligonucleotide
tgaagagacggtgaccattgtccc

SEQ ID NO: 588 - DNA, 16 bp, Artificial Sequence
  Description of Artificial Sequence Synthetic oligonucleotide
gacacctcgatcagcg

SEQ ID NO: 589 - DNA, 48 bp, Artificial Sequence
  Description of Artificial Sequence Synthetic oligonucleotide
gagtcattctcgacttgcggccgcacctaggacggtcagcttggtccc

SEQ ID NO: 590 - protein, 12 aa, Homo sapiens
QSYDRGFTGSMV

SEQ ID NO: 591 - protein, 12 aa, Homo sapiens
  MOD_RES (1)..(6): Any of the 20 naturally occurring amino acids
XXXXXXFTGSMV

SEQ ID NO: 592 - protein, 12 aa, Homo sapiens
  MOD_RES (4)..(9): Any of the 20 naturally occurring amino acids
QSYXXXXXXSMV

SEQ ID NO: 593 - protein, 12 aa, Homo sapiens
  MOD_RES (7)..(12): Any of the 20 naturally occurring amino acids
QSYDRGXXXXXX

SEQ ID NO: 594 - protein, 100 aa, Homo sapiens
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 595 - protein, 100 aa, Homo sapiens
EVQLVESGGGLVQPGGSLRLSCAASGFTFSDHYMSWVRQAQGKGLELVGLIRNKANSYTTEYAASVKGRLTISREDSKNTLYLQMSSLKTEDLAVYYCAR

SEQ ID NO: 596 - protein, 100 aa, Homo sapiens
EVQLVESGGGLVQPGGSLRLSCAASGFTFSDHYMSWVRQAQGKGLELVGLIRNKANSYTTEYAASVKGRLTISREDSKNTMYLQMSNLKTEDLAVYYCAR

SEQ ID NO: 597 - protein, 100 aa, Homo sapiens
EVQLLESGGGLVQPGGSLRLSCAASGFTFSDHYMSWVRQAQGKGLELVGLIRNKANSYTTEYAASVKGRLTISREDSKNTLYLQMSSLKTEDLAVYYCAR

SEQ ID NO: 598 - protein, 98 aa, Homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAK

SEQ ID NO: 599 - protein, 98 aa, Homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 600 - protein, 98 aa, Homo sapiens
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAK

SEQ ID NO: 601 - protein, 98 aa, Homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 602 - protein, 98 aa, Homo sapiens
QVQLLESGGGLVKPGGSLRLSCAASGFTFSDYYMSWIRQAPGKGLEWVSYISSSSSYTNYADSVKGRFTISRDNAKNSLYLQMNSLRAEDTAVYYCAR

SEQ ID NO: 603 - protein, 100 aa, Homo sapiens
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 604 - protein, 100 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 605 - protein, 100 aa, Homo sapiens
EVQLVESGGGLVKPGGSLRLSCAASGFTFSNAWMSWVRQAPGKGLEWVGRIESKTDGGTTDYAAPVKGRFTISRDDSKNTLYLQMNSLKTEDTAVYYCTT

SEQ ID NO: 606 - protein, 100 aa, Homo sapiens; identical to SEQ ID NO: 604

SEQ ID NO: 607 - protein, 100 aa, Homo sapiens
EVQLVESGGGLVKPGGSLRLSCAASGFTFSNAWMSWVRQAPGKGLEWVGRIKSKTDGGTTNYAAPVKGRFTISRDDSKNTLYLQMNSLKTEDTAVYYCTT

SEQ ID NO: 608 - protein, 100 aa, Homo sapiens
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTT

SEQ ID NO: 609 - protein, 100 aa, Homo sapiens
EVQLVESGGALVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 610 - protein, 98 aa, Homo sapiens
EVQLVESGGGLVQPGGSLRLSCPASGFTFSNHYMSWVRQAPGKGLEWVSYISGDSGYTNYADSVKGRFTISRDNANNSPYLQMNSLRAEDTAVYYCVK

SEQ ID NO: 611 - protein, 98 aa, Homo sapiens
EVQLVESGGGLVQPGGSLRLSCAASGFTFSNHYTSWVRQAPGKGLEWVSYSSGNSGYTNYADSVKGRFTISRDNAKNSLYLQMNSLRAEDTAVYYCVK

SEQ ID NO: 612 - protein, 98 aa, Homo sapiens
EVQLVESGGGLVQPGGSLRLSCAASGFTFSNSDMNWVHQAPGKGLEWVSGVSWNGSRTHYADSVKGRFIISRDNSRNTLYLQTNSLRAEDTAVYYCVR

SEQ ID NO: 613 - protein, 98 aa, Homo sapiens
EVQLVESGGGLVQPGGSLRLSCAASGFTFSNSDMNWARKAPGKGLEWVSGVSWNGSRTHYVDSVKRRFIISRDNSRNSLYLQKNRRRAEDMAVYYCVR

SEQ ID NO: 614 - protein, 98 aa, Homo sapiens
TVQLVESGGGLVEPGGSLRLSCAASGFTFSNSDMNWVRQAPGKGLEWVSGVSWNGSRTHYADSVKGRFIISRDNSRNFLYQQMNSLRPEDMAVYYCVR

SEQ ID NO: 615 - protein, 97 aa, Homo sapiens
EVHLVESGGGLVQPGGALRLSCAASGFTFSNYDMHWVRQATGKGLEWVSANGTAGDTYYPGSVKGRFTISRENAKNSLYLQMNSLRAGDTAVYYCAR

SEQ ID NO: 616 - protein, 97 aa, Homo sapiens
EVQLVETGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 617 - protein, 97 aa, Homo sapiens
EVQLVQSGGGLVHPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 618 - protein, 97 aa, Homo sapiens
EVQLVQSGGGLVQPGGSLRLSCAGSGFTFSSYAMHWVRQAPGKGLEWVSAIGTGGGTYYADSVKGRFTISRDNAKNSLYLQMNSLRAEDMAVYYCAR

SEQ ID NO: 619 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 620 - protein, 98 aa, Homo sapiens
EVQLVESGGGLVQPGGSLRLSCSASGFTFSSYAMHWVRQAPGKGLEYVSAISSNGGSTYYADSVKGRFTISRDNSKNTLYVQMSSLRAEDTAVYYCVK

SEQ ID NO: 621 - protein, 98 aa, Homo sapiens; identical to SEQ ID NO: 620

SEQ ID NO: 622 - protein, 98 aa, Homo sapiens
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCAR

SEQ ID NO: 623 - protein, 98 aa, Homo sapiens
EVQLLESGGGLVQPGGSLRLSCAASGFTFSSYAMSWVRQAPGKGLEWVSAISGSGGSTYYGDSVKGRFTISRDNSKNTLYLQMNSLRAEDTAVYYCAK

SEQ ID NO: 624 - protein, 98 aa, Homo sapiens
QVQLVESGGGVVQPGRSLRLSCAASGFTFSSYAMHWVRQAPGKGLEWVAVISYDGSNKYYTDSVKGRFTISRDNSKNTLYLQMNSLRAEDTAVYYCAR

SEQ ID NO: 625 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 626 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMSSLRAEDTAVYYCAR

SEQ ID NO: 627 - protein, 98 aa, Homo sapiens; identical to SEQ ID NO: 625

SEQ ID NO: 628 - protein, 98 aa, Homo sapiens; identical to SEQ ID NO: 625

SEQ ID NO: 629 - protein, 98 aa, Homo sapiens; identical to SEQ ID NO: 625

SEQ ID NO: 630 - protein, 98 aa, Homo sapiens; identical to SEQ ID NO: 625

SEQ ID NO: 631 - protein, 98 aa, Homo sapiens; identical to SEQ ID NO: 625

SEQ ID NO: 632 - protein, 98 aa, Homo sapiens; identical to SEQ ID NO: 625

SEQ ID NO: 633 - protein, 98 aa, Homo sapiens
QVQLVESGGGLVQPGGSLRLSCSASGFTFSSYAMHWVRQAPGKGLEYVSAISSNGGSTYYADSVKGRFTISRDNSKNTLYVQMSSLRAEDTAVYYCVK

SEQ ID NO: 634 - protein, 98 aa, Homo sapiens
QVQLVESGGGLVQPGGSLRLSCSASGFTFSSYAMHWVRQAPGKGLEYVSAISSNGGSTYYADSVKGRFTISRDNSKNTLYLQMNSLRAEDTAVYYCAR

SEQ ID NO: 635 - protein, 98 aa, Homo sapiens
QVQLVESGGGVVQPGRSLRLSCAASGFTFSSYAMHWVRQAPGKGLEWVAVISYDGSNKYYADSVKGRFAISRDNSKNTLYLQMNSLRAEDTAVYYCAR

SEQ ID NO: 636 - protein, 98 aa, Homo sapiens; identical to SEQ ID NO: 625

SEQ ID NO: 637 - protein, 98 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAK

SEQ ID NO: 638 - protein, 97 aa, Homo sapiens
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 639 - protein, 98 aa, Homo sapiens
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCAR

SEQ ID NO: 640 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRLRARLCITVRE

SEQ ID NO: 641 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAR

SEQ ID NO: 642 - protein, 98 aa, Homo sapiens; identical to SEQ ID NO: 641

SEQ ID NO: 643 - protein, 98 aa, Homo sapiens; identical to SEQ ID NO: 641

SEQ ID NO: 644 - protein, 98 aa, Homo sapiens; identical to SEQ ID NO: 641

SEQ ID NO: 645 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNRLYLQMNSLRAEDTAVYYCAR

SEQ ID NO: 646 - protein, 98 aa, Homo sapiens; identical to SEQ ID NO: 641

SEQ ID NO: 647 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 648 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEGTAVYYCAR

SEQ ID NO: 649 - protein, 98 aa, Homo sapiens
QVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCAK

SEQ ID NO: 650 - protein, 98 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 651 - protein, 98 aa, Homo sapiens; identical to SEQ ID NO: 647

SEQ ID NO: 652 - protein, 98 aa, Homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAK

SEQ ID NO: 653 - protein, 95 aa, Homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVRK

SEQ ID NO: 654 - protein, 98 aa, Homo sapiens; identical to SEQ ID NO: 641

SEQ ID NO: 655 - protein, 98 aa, Homo sapiens
QVQLVESGGGVVQPGRSLRLSCAASGFTFSSYGMHWVRQAPGKGLEWVAVIWYDGSNKYYADSAKGRFTISRDNSTNTLFLQMNSLRAEDTAVYYCAR

SEQ ID NO: 656 - protein, 98 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRDEDTAVYYCAR

SEQ ID NO: 657 - protein, 98 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 658 - protein, 97 aa, Homo sapiens
EVQLVESGGGLVKPGGSLRLSCAASGFTFSSYSMNWVRQAPGKGLEWVSSISSSSYIYYADSVKGRFTISRDNAKNSLYLQMNSLRAEDTAVYYCAR

SEQ ID NO: 659 - protein, 98 aa, Homo sapiens; identical to SEQ ID NO: 657

SEQ ID NO: 660 - protein, 98 aa, Homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAR

SEQ ID NO: 661 - protein, 97 aa, Homo sapiens
EDQLVESGGGLVQPGGSLRPSCAASGFAFSSYVLHWVRRAPGKGPEWVSAIGTGGDTYYADSVMGRFTISRDNAKKSLYLQMNSLIAEDMAVYYCAR

SEQ ID NO: 662 - protein, 98 aa, Homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 663 - protein, 98 aa, Homo sapiens; identical to SEQ ID NO: 662

SEQ ID NO: 664 - protein, 98 aa, Homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 665 - protein, 98 aa, Homo sapiens
EVQLVESGGGLVQPGGSLRLSCAASGFTFSSYWMHWVRQAPGKGLVWVSRINSDGSSTSYADSMKGQFTISRDNAKNTLYLQMNSLRAEDMAVYYCTR

SEQ ID NO: 666 - protein, 98 aa, Homo sapiens; identical to SEQ ID NO: 664

SEQ ID NO: 667 - protein, 98 aa, Homo sapiens
QVQLVQSGGGVVQPGRSLRLSCAASGFTFSSYGMHWVRQAPGKGLEWVAFIRYDGSNKYYADSVKGRFTISRDNSKNTLYLQMKSLRAEDTAVYYCTT

SEQ ID NO: 668 - protein, 98 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSA

SEQ ID NO: 669 - protein, 98 aa, Homo sapiens
QSVLTQPPSVSAAPGQKVTISCSGSSSDMGNYAVSWYQQLPGTAPKLLIYENNKRPSGIPDRFSGSKSGTSATLGITGLWPEDEADYYCLAWDTSPRA

SEQ ID NO: 670 - protein, 98 aa, Homo sapiens
QSVLTQPPSASGTPGQRVTISCSGSSSNIGSNTVNWYQQLPGTAPKLLIYSNNQRPSGVPDRFSGSKSGTSASLAISGLQSEDEADYYCAAWDDSLNG

SEQ ID NO: 671 - protein, 98 aa, Homo sapiens
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSG

SEQ ID NO: 672 - protein, 98 aa, Homo sapiens
QSVLTQPPSVSEAPRQRVTISCSGSSSNIGNNAVNWYQQLPGKAPKLLIYYDDLLPSGVSDRFSGSKSGTSASLAISGLQSEDEADYYCAAWDDSLNG

SEQ ID NO: 673 - protein, 99 aa, Homo sapiens
QSVLTQPPSVSGAPGQRVTISCTGSSSNIGAGYVVHWYQQLPGTAPKLLIYGNSNRPSGVPDQFSGSKSGTSASLAITGLQSEDEADYYCKAWDNSLNA

SEQ ID NO: 674 - protein, 99 aa, Homo sapiens
QSVVTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSG

SEQ ID NO: 675 - protein, 98 aa, Homo sapiens
SYVLTQPPSVSGTPGQRVTISCSGGRSNIGSNTVKWYQQLPGTAPKLLIYGNDQRPSGVPDRFSGSKSGTSASLAITGVQAEDEADYYCQSYDSSLRG